(19) United States
(12) Patent Application Publication
(10) Pub. No.: US 2016/0310427 A1
(43) Pub. Date: Oct. 27, 2016

(54) TAMPER-RESISTANT FIXED DOSE COMBINATION PROVIDING FAST RELEASE OF TWO DRUGS FROM PARTICLES AND A POWDER

(71) Applicant: GRÜNENTHAL GMBH, Aachen (DE)

(72) Inventors: KLAUS WENING, Koln (DE); LUTZ BARNSCHEID, Monchengladbach (DE); ANJA GEISSLER, Stolberg (DE); JANA DENKER, Bornheim (DE); JESSICA REDMER, Monchengladbach (DE)

(73) Assignee: GRÜNENTHAL GMBH, Aachen (DE)

(21) Appl. No.: 15/135,594

(22) Filed: Apr. 22, 2016

(30) Foreign Application Priority Data
Apr. 24, 2015 (EP) 15 165 067.8

(51) Int. Cl. A61K 9/16; A61K 31/137; A61K 9/48; A61K 31/485; A61K 31/167

(52) U.S. Cl. CPC A61K 9/1641; A61K 9/1617; A61K 9/1635; A61K 31/485; A61K 31/167; A61K 9/1694; A61K 9/4808; A61K 9/4825; A61K 9/1652; A61K 31/137

(57) ABSTRACT

A tamper-resistant pharmaceutical dosage form comprising two pharmacologically active ingredients, wherein the dosage form provides under in vitro conditions fast release, preferably immediate release according to Ph. Eur., of both pharmacologically active ingredients. The dosage form is useful for pharmaceutical combination therapy that is achieved by administering dosage forms containing more than one pharmacologically active ingredient as fixed-dose combinations.

This application claims priority of European Patent Application No. 15 165 067.8, filed on Apr. 24, 2015, the entire contents of which is incorporated herein by reference.

The invention relates to a tamper-resistant pharmaceutical dosage form comprising two pharmacologically active ingredients, wherein the dosage form provides under in vitro conditions fast release, preferably immediate release according to Ph. Eur., of both pharmacologically active ingredients. The dosage form according to the invention is useful for pharmaceutical combination therapy that is achieved by administering dosage forms containing more than one pharmacologically active ingredient as fixed-dose combinations.

In combination therapy, the combined drugs typically have different targets (multi-target combinations). The scientific rationale behind multi-target combinations is the therapeutic benefit which could not be achieved by the individual drugs alone. The drugs of the combination act together additively or even synergistically and cooperate to achieve a completeness of the desired therapeutic effect. For example, a major advantage of using multi-target combinations in pain therapy is that the drugs, e.g. analgesics, are able to act on more signaling cascades involved in pain than most single analgesics, without adding more undesired side effects to the therapy. On the contrary, as the individual dosages of each drug in the combination can often be reduced in view of the presence of the additional drug within the combination, a reduction of undesired side effects may be achieved.

A large number of drugs have a potential for being abused or misused, i.e. they can be used to produce effects which are not consistent with their intended use. Thus, e.g. opioids which exhibit an excellent efficacy in controlling moderate and severe pain are frequently abused to induce euphoric states similar to being intoxicated. In particular, drugs which have a psychotropic effect are abused accordingly.

To enable abuse, the corresponding dosage forms such as tablets or capsules are crushed, for example ground by the abuser, the drug is extracted from the thus obtained powder using a preferably aqueous liquid and after being optionally filtered, the resultant solution is administered parenterally, in particular intravenously. This type of administration results in an even faster diffusion of the drug compared to the oral abuse, with the result desired by the abuser, namely the kick. This kick or these intoxication-like, euphoric states are also reached if the powdered dosage form is administered nasally, i.e. is sniffed.

Various concepts for the avoidance of drug abuse have been developed.

It has been proposed to incorporate in dosage forms aversive agents and/or antagonists in a manner so that they only produce their aversive and/or antagonizing effects when the dosage forms are tampered with. However, the presence of such aversive agents, e.g. bitter substances, irritants, colorants, emetics, and the like is principally not desirable and there is a need to provide sufficient tamper-resistance without relying on aversive agents and/or antagonists.

Another concept to prevent abuse relies on the mechanical properties of the pharmaceutical dosage forms, particularly an increased breaking strength (resistance to crushing). The mechanical properties, particularly the high breaking strength of these pharmaceutical dosage forms renders them tamper-resistant. The major advantage of such pharmaceutical dosage forms is that comminuting, particularly pulverization, by conventional means, such as grinding in a mortar or fracturing by means of a hammer, is impossible or at least substantially impeded. Thus, the pulverization, necessary for abuse of the dosage forms, by the means that are usually available to a potential abuser is prevented or at least complicated. Such pharmaceutical dosage forms are useful for avoiding drug abuse of the drug contained therein, as they may not be powdered by conventional means and thus, cannot be administered in powdered form, e.g. nasally. In the context of such break resistant pharmaceutical dosage forms it can be referred to, e.g., WO 2005/016313, WO 2005/016314, WO 2005/063214, WO 2005/102286, WO 2006/002883, WO 2006/002884, WO 2006/002886, WO 2006/082097, WO 2006/082099, WO 2008/107149, WO 2009/092601, WO 2011/009603, WO 2011/009602, WO 2009/135680, WO 2011/095314, WO 2012/028317, WO 2012/028318, WO 2012/028319, WO 2011/009604, WO 2013/017242, WO 2013/017234, WO 2013/050539, WO 2013/127830, WO 2013/072395, WO 2013/127831, WO 2013/156453, WO 2013/167735, WO 2015/004245, WO 2014/191396, and WO 2014/191397.

Still another concept to prevent abuse relies on the presence of auxiliary substances that increase the viscosity of the resultant composition when the dosage forms are tampered with, e.g. when they are subjected to liquids in order to prepare formulations for parenteral administration, e.g. intravenous injection. Said auxiliary substances increase the viscosity of the resultant compositions to such an extent that the liquids cannot be drawn-up in syringes. While it may be possible to extract the drug from the dosage form at least to a certain extent, the extract is not useful for subsequent abuse.

WO 2008/033523 discloses a pharmaceutical composition that may include a granulate which may at least include one active pharmaceutical ingredient susceptible to abuse. The particle contains both an alcohol soluble and alcohol insoluble and at least partially water soluble material. Both materials are granulated in the presence of alcohol and water. The granulate may also include a coating exhibiting crush resistance. Material deposition on the granule is performed using an alcohol based solvent.

WO 2008/107149 discloses multiparticulate dosage forms with impeded abuse containing, one or more active substances having abuse potential, at least one synthetic or natural polymer, and at least one disintegrant, with the individual particles of the pharmaceutical dosage form having a breaking strength of at least 500 N and a release of the active substance of at least 75% after 45 minutes. The exemplified capsules provide rapid release of the pharmacologically active compound. The disintegrant is preferably not contained in the particulates. When it is contained in the particulates, its content is rather low. The reference does not contain any information that besides its disintegrating effect a disintegrant may have any beneficial effect with respect to tamper resistance such as resistance against solvent extraction.

WO 2010/140007 discloses dosage forms comprising melt-extruded particles comprising a drug, wherein said melt-extruded particles are present as a discontinuous phase in a matrix. The dosage forms provide prolonged release of the drug.

WO 2012/061779 relates in one embodiment to abuse-deterrent drug formulations comprising a plurality of discrete domains uniformly dispersed in a pharmaceutically acceptable matrix, wherein said domains have high fracture toughness and comprise at least one polymer and at least one abuse-relevant drug. In another embodiment, the present invention relates to a formulation comprising a plurality of discrete mechanically reinforcing particles uniformly dispersed in a pharmaceutically acceptable matrix, wherein said matrix has high fracture toughness and comprises at least one polymer and at least one active agent, at least one abuse-relevant drug or a combination of at least one active agent and at least one abuse-relevant drug.

WO 2013/017242 and WO 2013/017234 disclose a tamper-resistant tablet comprising a matrix material in an amount of more than one third of the total weight of the tablet; and a plurality of particulates in an amount of less than two thirds of the total weight of the tablet; wherein said particulates comprise a pharmacologically active compound and a polyalkylene oxide; and form a discontinuous phase within the matrix material. The matrix material may comprise a disintegrant. The reference does not contain any information that besides its disintegrating effect a disintegrant may have any beneficial effect with respect to tamper resistance such as resistance against solvent extraction.

WO 2013/030177 relates to an abuse resistant tablet formulation based on paracetamol and oxycodone.

WO 2014/190440 relates to an immediate release orally administrable abuse-deterrent pharmaceutical formulation comprising: at least one pharmaceutically active ingredient susceptible to abuse; at least one gelling polymeric compound selected from the group consisting of: polysaccharides, sugars, sugar derived alcohols, starches, starch derivatives, cellulose derivatives, Carrageenan, pectin, sodium alginate, gellan gum, xanthan gum, poloxamer, carbopol, polyox, povidone, hydroxypropylmethylcellulose, hypermellose, and combinations thereof; at least one disintegrant and optionally at least one surfactant, wherein said formulation exhibit properties related to deterring the abuse, via injection or nasal inhalation when being tampered and exposed to aqueous, alcoholic, acidic and basic media.

WO 2014/191396 relates to a pharmaceutical dosage form comprising (i) at least one formed segment (S₁), which contains a first pharmacologically active ingredient (A₁) and provides prolonged release thereof, and (ii) at least one further segment (S₂), which contains a second pharmacologically active ingredient (A₂) and provides immediate release thereof, wherein the at least one formed segment (S₁) exhibits a higher breaking strength than the at least one further segment (S₂) and the at least one formed segment (S₁) exhibits a breaking strength of more than 500 N.

US 2003/092724 relates to oral tablet compositions which include an immediate release portion having an opioid analgesic and a non-opioid analgesic, providing for a rapid onset of therapeutic effect, and a sustained release portion of an opioid analgesic and a non-opioid analgesic, providing for a relatively longer duration of therapeutic effect.

US 2007/0292508 discloses orally disintegrating dosage forms comprising lipid coated substrates and silicified excipients.

US 2010/0092553 discloses solid multiparticle oral pharmaceutical forms whose composition and structure make it possible to avoid misuse. The microparticles have an extremely thick coating layer which assures the modified release of the drug and simultaneously imparts crushing resistance to the coated microparticles so as to avoid misuse.

US 2012/0077879 discloses a process for preparing solid dosage forms that contain poorly compressible therapeutic compound. The process, for example, provides for the use of an extruder, especially a twin screw extruder, to melt granulate a therapeutic compound(s) with a granulation excipient.

US 2013/289062 relates to an abuse deterrent dosage form of opioid analgesics, wherein an analgesically effective amount of opioid analgesic is combined with a polymer to form a matrix.

The properties of conventional tamper-resistant dosage forms are not satisfactory in every respect. The requirements for tamper-resistant dosage forms that nowadays need to be satisfied are complex and sometimes are difficult to be combined and arranged with one another. While a certain measure may improve tamper-resistance in a certain aspect, the same measure may deteriorate tamper-resistance in another aspect or otherwise may have a detrimental effect on the properties of the dosage forms.

When trying to tamper the dosage forms, e.g. in order to prepare formulations suitable for abuse by intravenous administration, the liquid part of the formulations that can be separated from the remainder by means of a syringe should be as less as possible. When trying to crush the dosage forms, e.g. in order to prepare formulations suitable for abuse by nasal administration, the particle size of the crushed powder, if any, should be as large as possible such that absorption through the mucosa proceeds slowly, if at all.

Drug release and disintegration times of dosage forms providing immediate drug release require a design that substantially differs from the design of dosage forms providing prolonged drug release (e.g. sustained release, extended release, delayed release, and the like). Dosage forms providing immediate release are typically customized for frequent administration so that they do not need to contain the entire daily dosage of the drug. However, auxiliary substances that are added in order to achieve tamper-resistance, e.g. an increased breaking strength and/or an increased viscosity after extraction in suitable liquids, often have a retardant effect on drug release so that tamper-resistance on the one hand and immediate drug release on the other hand may antagonize one another and need to be balanced. In consequence, tamper-resistant dosage forms providing immediate drug release are typically multiparticulate whereas the particles are of intermediate size. On the one hand, the particles are sufficiently large to contain sufficient amounts of auxiliary substances to render them tamper-resistant. On the other hand, the particles are sufficiently small to enable immediate drug release.

Another aspect that must not be neglected for tamper-resistant dosage forms is patient compliance. In this regard, especially the overall volume of oral dosage forms must not exceed a certain limit so that they can be swallowed by the patients. The volume of a dosage forms is substantially influenced by the potency/efficacy of the drug. If the daily dose amounts to a few micrograms only, small dosage forms can be manufactured. If the daily dosage amounts to several hundred milligrams, however, the dosage form becomes larger and larger. Furthermore, the volume of a dosage form is substantially influenced by the presence of auxiliary substances that contribute to tamper-resistance and/or the desired release kinetics. Conventional dosage forms having substantial volume and size are often fragmented or disassembled prior to administration for the ease of swallowing. When dealing with tamper-resistant dosage forms, however, this is not always possible because the concept of avoiding drug abuse may rely on the prevention of such fragmentation. In tamper-resistant dosage forms providing prolonged drug release, the underlying concept of retarding drug release may not tolerate fragmentation either.

While the above aspects principally apply to every tamper-resistant dosage form, additional problems arise when the tamper-resistant dosage form contains more than a single drug. Under these circumstances, every drug may require its own formulation in order to achieve the desired release kinetics, tamper-resistance, storage stability, and further properties. Satisfying all these requirements in a single dosage can become particularly difficult and complex, especially when the drugs have a different potency/efficacy so that content of one drug needs to a few milligrams only, whereas the content of the other drug(s) needs to be in the range of several hundred milligrams. For example, a dosage form containing suitable dosages of a combination of hydrocodone and acetaminophen may contain e.g. about 30-times more acetaminophen than hydrocodone.

Furthermore, as tamper-resistant dosage forms usually compete on the market with their non-tamper-resistant counterparts containing the same drug(s), the process for preparing the tamper-resistant dosage forms must be efficient, straightforward and easy. Otherwise, the tamper-resistant dosage forms become so expensive that in spite of their advantages with respect to the avoidance of drug abuse and misuse, they have problems to be launched and established on the market. However, the manufacture of tamper-resistant dosage forms is likely always more laborious and thus more expensive than the manufacture of their non-tamper-resistant counterparts. This economic disadvantage of tamper-resistant dosage forms may at least partially be compensated when the individual components are useful for the preparation of a multitude of tamper-resistant dosage forms. For example, when a specific tamper-resistant particle containing a specific drug and exhibiting specific release kinetics for said drug can be combined with various other drugs in different products, said tamper-resistant particle can be provided as bulk material. Another advantage of particulate dosage forms is that particles of different properties, containing the same drug or different drugs, can be combined with one another in the meaning of a kit, e.g. in order to achieve multimodal drug release.

It is an object of the invention to provide tamper-resistant pharmaceutical dosage forms that provide fast release of two pharmacologically active compounds contained therein and that have advantages compared to the tamper-resistant pharmaceutical dosage forms of the prior art.

This object has been achieved by the subject-matter of the patent claims.

A first aspect of the invention relates to a tamper-resistant pharmaceutical dosage form comprising a pharmacologically active ingredient a having a psychotropic effect and a pharmacologically active ingredient b;

wherein the dosage form provides under in vitro conditions fast release, preferably immediate release according to Ph. Eur., of the pharmacologically active ingredient a and fast release, preferably immediate release according to Ph. Eur., of the pharmacologically active ingredient b; and wherein
at least a portion of the pharmacologically active ingredient a, preferably its total amount, is contained in one or more particles A which comprise a polymer matrix in which the pharmacologically active ingredient a is embedded; and
at least a portion of the pharmacologically active ingredient b is contained outside the one or more particles A in form of a powder.

It has been surprisingly found that tamper-resistant dosage forms can be provided that on the one hand provide fast release, preferably immediate release of the pharmacologically active ingredient a as well as of the pharmacologically active ingredient b and that on the other hand provide improved tamper-resistance.

Further, it has been surprisingly found that the dosage forms according to the invention provide a good balance of tamper-resistance and other properties that are desirable for dosage form providing fast release of two pharmacologically active ingredients such as patient compliance.

Still further, it has been surprisingly found that the dosage forms according to the invention are useful for combinations of two or more pharmacologically active ingredients having substantially different potency/efficacy such that they must be contained in substantially different quantities in order to provide the desired therapeutic effect.

Yet further, it has been surprisingly found that the pharmacologically active ingredient that is present in greater total quantity can be divided into two or more portions that are contained in different compartments of the dosage form without resulting in a bi- or trimodal release thereof. For example, it has been found that a portion b_{A} of the pharmacologically active ingredient b may be contained together with pharmacologically active ingredient a in the one or more particles A, whereas another portion b_{P} of the pharmacologically active ingredient b may be present outside particles A in form of a powder. It has been found that such dosage form according to the invention still provides fast release of the pharmacologically active ingredient b, although the mechanism of release from particle(s) A differs from the mechanism of release from the powder.

Furthermore, the dosage forms according to the invention can be manufactured by efficient, straightforward and easy processes. As the dosage forms according to the invention are composed of individual components or units that in the course of manufacture are preferably prepared separately of one another and then finally combined to provide the dosage form according to the invention, said individual components are useful for the preparation of a multitude of tamper-resistant dosage forms making the process even more cost efficient.

The concept underlying the dosage forms according to the invention provides a high degree in flexibility concerning dosage, release profile, tamper-resistance, patient compliance, ease of manufacture and the like. The dosage forms according to the invention can be prepared from a variety of components that are separately prepared. Specific selection of specific components from said variety of components allows for tailoring dosage forms satisfying a large variety of different requirements. For example, it is possible to make available a variety of three different types of particle(s) A that differ e.g. in their content of pharmacologically active compound a. When manufacturing pharmaceutical dosage forms according to the invention having a predetermined total dosage of pharmacologically active ingredient a, one may select different combinations of particles A₁, A₂ and A₃ in order to achieve said total dosage of pharmacologically active ingredient a. For example, particles A₁ may contain a dosage of 0.25 mg, particles A₂ may contain a dosage of 1.50 mg, and particles A₃ may contain a dosage of 3.50 mg, such that a total dosage of e.g. 5.00 mg pharmacologically active ingredient a can be achieved by
20 particles A₁;
2 particles A₁ in combination with 3 particles A₂;
1 particle A₂ in combination with 1 particle A₃; or
6 particles A₁ in combination with 1 particle A₃.

Another advantage of the concept underlying the dosage forms according to the invention is that nearly every combination may be either filled into capsules or may be compressed into tablets. This flexibility has particular advantages when providing tamper-resistant products that need to satisfy the confidence requirements with respect to the marketing authorization for the initial non-tamper-resistant product. Thus, the present invention makes available at a high degree of flexibility tamper-resistant counterparts to existent non-tamper-resistant products. If in initial tests the confidence intervals are not met, the present invention provides easy and predictable measures for slightly altering the properties of the dosage form in order to meet the confidence requirements.

Further, providing the dosage forms in form of capsules has additional advantages with respect to patient compliance. Capsules are also particularly useful for pediatric applications, especially sprinkle capsules.

### BRIEF DESCRIPTION OF THE DRAWINGS

FIG. 1 illustrates the preferred behavior of the particle(s) contained in the dosage form according to the invention when being subjected to a breaking strength test, in particular their deformability.

FIG. 2 illustrates the behavior of conventional particle(s) when being subjected to a breaking strength test.

FIG. 3 shows the in vitro release profiles of exemplified dosage forms with respect to the release of hydrocodone (pharmacologically active ingredient a).

FIG. 4 shows the in vitro release profiles of exemplified dosage forms with respect to the release of acetaminophen (pharmacologically active ingredient b).

FIG. 5 illustrates preferred embodiments of pharmaceutical dosage forms according to the invention. FIG. 5A illustrates a capsule comprising a multitude of particles A (1) and pharmacologically active ingredient b that is contained outside the particles A in form of a powder (2). Particles A (1) may additionally comprise a portion b_{A} of pharmacologically active ingredient b and/or a coating comprising a portion be of pharmacologically active ingredient b. The capsule according to FIG. 5B additionally comprises a portion b_{B} of pharmacologically active ingredient b contained in particles B (3), whereas the capsule according to FIG. 5C additionally comprises a portion b_{G} of pharmacologically active ingredient b in form of granules (4).

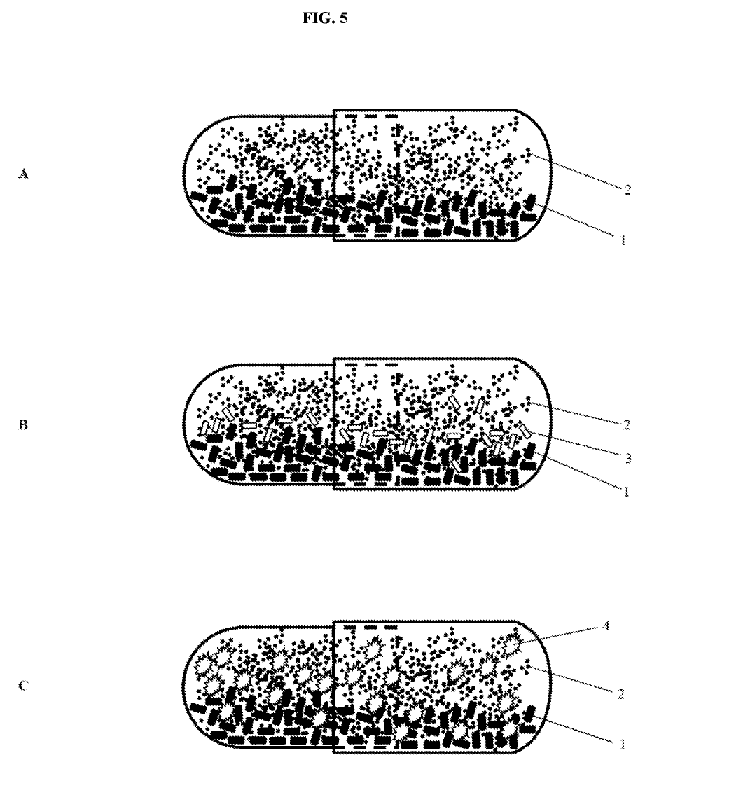

As used herein, the term “pharmaceutical dosage form” or “dosage form” refers to a pharmaceutical entity comprising a pharmacologically active ingredient a as well as a pharmacologically active ingredient b and which is actually administered to, or taken by, a patient, preferably orally.

Preferably, the dosage form according to the invention is a capsule or a tablet. When the dosage form contains at least a portion b_{P} of the pharmacologically active ingredient b in form of a powder, the dosage form is preferably a capsule, as it is difficult to formulate a (non-compacted) powder in form of tablet.

In a preferred embodiment, when the dosage form is a capsule, it is preferably a sprinkle capsule or a multitude of sprinkle capsules. The capsule may comprise the particles and all excipients in form of a loose filling, i.e. an homogeneous mixture, or in form of layers (layered capsule filling).

In another preferred embodiment, when the dosage form is a tablet, the tablet may comprise the particle(s) A and the optionally present particle(s) B in an outer matrix material with homogeneous distribution or in form of a mantle tablet.

The dosage from comprises particle(s) of a first type, referred to as “particles(s) A” and optionally additional particle(s) of a second type, referred to as “particle(s) B”. The particle(s) A, the optionally present particle(s) B, and/or the dosage form as such may be film-coated. When at least a portion b_{P} of the pharmacologically active ingredient b is present in form of a powder, i.e. a loose plurality of fine pieces of material or a heap of loose material, the dosage form according to the invention is preferably a capsule.

The dosage form according to the invention comprises one or more particles A and optionally, additionally one or more particles B. In the following, it is referred to “particles(s) A” and “particle(s) B” in order to express that the number of particles in each case may be independently one or more. When it is referred to “particle(s)”, the respective embodiment independently applies to both, to particle(s) A and to optionally present particle(s) B.

The dosage form according to the invention may be compressed or molded in its manufacture, and it may be of almost any size, shape, weight, and color. Most dosage forms are intended to be swallowed as a whole and accordingly, preferred dosage forms according to the invention are designed for oral administration. However, alternatively dosage forms may be dissolved in the mouth, chewed, or dissolved or dispersed in liquid or meal before swallowing, and some may be placed in a body cavity. Thus, the dosage form according to the invention may alternatively be adapted for buccal, lingual, rectal or vaginal administration. Implants are also possible.

In a preferred embodiment, the dosage form according to the invention preferably can be regarded as a MUPS formulation (multiple unit pellet system). In a preferred embodiment, the dosage form according to the invention is monolithic. In another preferred embodiment, the dosage form according to the invention is not monolithic. In this regard, monolithic preferably means that the dosage form is formed or composed of material without joints or seams or consists of or constitutes a single unit.

In a preferred embodiment, the dosage form according to the invention contains all ingredients in a dense compact unit which in comparison to capsules has a comparatively high density. In another preferred embodiment, the dosage form according to the invention contains all ingredients in a capsule which in comparison to dense compact unit has a comparatively low density.

An advantage of the dosage forms according to the invention is that upon manufacture the particle(s) A may be mixed with excipients in different amounts to thereby produce dosage forms of different strengths. Another advantage of the dosage forms according to the invention is that upon manufacture the different particle(s) A, i.e. particles A having a different constitution, may be mixed with one another to thereby produce dosage forms of different properties, e.g. different release rates, different pharmacologically active ingredients a, and the like.

The dosage form according to the invention is characterized by excellent storage stability. Preferably, after storage for 12 months, 9 months, 6 months, 3 months, 2 months, or 4 weeks at 40° C. and 75% rel. humidity, the content of pharmacologically active ingredient a and the content of pharmacologically active ingredient b independently of one another amounts to at least 98.0%, more preferably at least 98.5%, still more preferably at least 99.0%, yet more preferably at least 99.2%, most preferably at least 99.4% and in particular at least 99.6%, of its original content before storage. Suitable methods for measuring the content of the pharmacologically active ingredient a and of pharmacologically active ingredient b in the dosage form are known to the skilled artisan. In this regard it is referred to the Eur. Ph. or the USP, especially to reversed phase HPLC analysis. Preferably, the dosage form is stored in closed, preferably sealed containers.

The dosage form according to the invention has preferably a total weight in the range of 0.01 to 1.5 g, more preferably in the range of 0.05 to 1.2 g, still more preferably in the range of 0.1 g to 1.0 g, yet more preferably in the range of 0.2 g to 0.9 g, and most preferably in the range of 0.3 g to 0.8 g. In a preferred embodiment, the total weight of the dosage form is within the range of 500±450 mg, more preferably 500±300 mg, still more preferably 500±200 mg, yet more preferably 500±150 mg, most preferably 500±100 mg, and in particular 500±50 mg. In another preferred embodiment, the total weight of the dosage form is within the range of 600±450 mg, more preferably 600±300 mg, still more preferably 600±200 mg, yet more preferably 600±150 mg, most preferably 600±100 mg, and in particular 600±50 mg. In still another preferred embodiment, the total weight of the dosage form is within the range of 700±450 mg, more preferably 700±300 mg, still more preferably 700±200 mg, yet more preferably 700±150 mg, most preferably 700±100 mg, and in particular 700±50 mg. In yet another preferred embodiment, the total weight of the dosage form is within the range of 800±450 mg, more preferably 800±300 mg, still more preferably 800±200 mg, yet more preferably 800±150 mg, most preferably 800±100 mg, and in particular 800±50 mg.

In a preferred embodiment, the dosage form according to the invention is a round dosage form, preferably having a diameter of e.g. 11 mm or 13 mm. Dosage forms of this embodiment preferably have a diameter in the range of 1 mm to 30 mm, in particular in the range of 2 mm to 25 mm, more in particular 5 mm to 23 mm, even more in particular 7 mm to 13 mm; and a thickness in the range of 1.0 mm to 12 mm, in particular in the range of 2.0 mm to 10 mm, even more in particular from 3.0 mm to 9.0 mm, even further in particular from 4.0 mm to 8.0 mm.

In another preferred embodiment, the dosage form according to the invention is an oblong dosage form, preferably having a length of e.g. 17 mm and a width of e.g. 7 mm. In preferred embodiments, the dosage form according to the invention has a length of e.g. 22 mm and a width of e.g. 7 mm; or a length of 23 mm and a width of 7 mm; whereas these embodiments are particularly preferred for capsules. Dosage forms of this embodiment preferably have a lengthwise extension (longitudinal extension) of 1 mm to 30 mm, in particular in the range of 2 mm to 25 mm, more in particular 5 mm to 23 mm, even more in particular 7 mm to 20 mm; a width in the range of 1 mm to 30 mm, in particular in the range of 2 mm to 25 mm, more in particular 5 mm to 23 mm, even more in particular 7 mm to 13 mm; and a thickness in the range of 1.0 mm to 12 mm, in particular in the range of 2.0 mm to 10 mm, even more in particular from 3.0 mm to 9.0 mm, even further in particular from 4.0 mm to 8.0 mm.

In a preferred embodiment, the dosage form according to the invention is not film coated.

In another preferred embodiment, the dosage form according to the invention is provided, partially or completely, with a conventional coating. The dosage forms according to the invention are preferably film coated with conventional film coating compositions. Suitable coating materials are commercially available, e.g. under the trademarks Opadry®, Opaglos® and Eudragit®.

Examples of suitable materials include cellulose esters and cellulose ethers, such as methylcellulose (MC), hydroxypropylmethylcellulose (HPMC), hydroxypropylcellulose (HPC), hydroxyethylcellulose (HEC), sodium carboxymethylcellulose (Na-CMC), poly(meth)acrylates, such as aminoalkylmethacrylate copolymers, methacrylic acid methylmethacrylate copolymers, methacrylic acid methylmethacrylate copolymers; vinyl polymers, such as polyvinylpyrrolidone, polyvinyl alcohol, polyvinylacetate; and natural film formers.

In a particularly preferred embodiment, the coating is water-soluble. In a preferred embodiment, the coating is based on polyvinyl alcohol, such as polyvinyl alcohol-partially hydrolyzed, and may additionally contain polyethylene glycol, such as macrogol 3350, and/or pigments. In another preferred embodiment, the coating is based on hydroxypropylmethylcellulose, preferably hypromellose type 2910 having a viscosity of 3 to 15 mPas.

The coating can be resistant to gastric juices and dissolve as a function of the pH value of the release environment. By means of this coating, it is possible to ensure that the dosage form according to the invention passes through the stomach undissolved and the active compound is only released in the intestines. The coating which is resistant to gastric juices preferably dissolves at a pH value of between 5 and 7.5.

The coating can also be applied e.g. to improve the aesthetic impression and/or the taste of the dosage forms and the ease with which they can be swallowed. Coating the dosage forms according to the invention can also serve other purposes, e.g. improving stability and shelf-life. Suitable coating formulations comprise a film forming polymer such as, for example, polyvinyl alcohol or hydroxypropyl methylcellulose, e.g. hypromellose, a plasticizer such as, for example, a glycol, e.g. propylene glycol or polyethylene glycol, an opacifier, such as, for example, titanium dioxide, and a film smoothener, such as, for example, talc. Suitable coating solvents are water as well as organic solvents. Examples of organic solvents are alcohols, e.g. ethanol or isopropanol, ketones, e.g. acetone, or halogenated hydrocarbons, e.g. methylene chloride. Coated dosage forms according to the invention are preferably prepared by first making the cores and subsequently coating said cores using conventional techniques, such as coating in a coating pan.

The subjects to which the dosage forms according to the invention can be administered are not particularly limited. Preferably, the subjects are animals, more preferably human beings.

The tamper-resistant dosage form according to the invention comprises particle(s) A which comprise the pharmacologically active ingredient a. Preferably, the particle(s) A contain the total amount of pharmacologically active ingredient a that is contained in the dosage form according to the invention, i.e. the dosage form according to the invention preferably does not contain pharmacologically active ingredient a outside particle(s) A. The particle(s) A contain at least a pharmacologically active ingredient a and a polymer matrix that preferably comprises a polyalkylene oxide. Preferably, however, the particle(s) A contain additional pharmaceutical excipients such as disintegrants, antioxidants and plasticizers. The pharmacologically active ingredient a is embedded, preferably dispersed in a polymer matrix preferably comprising a polyalkylene oxide.

The pharmacologically active ingredient a is not particularly limited.

In a preferred embodiment, the particle(s) A and the dosage form, respectively, contain only a single pharmacologically active ingredient a besides pharmacologically active ingredient b. In another preferred embodiment, the particle(s) A and the dosage form, respectively, contain a combination of two or more pharmacologically active ingredient a besides pharmacologically active ingredient b.

Preferably, pharmacologically active ingredient a is an active ingredient with potential for being abused. Active ingredients with potential for being abused are known to the person skilled in the art and comprise e.g. tranquillizers, stimulants, barbiturates, narcotics, opioids or opioid derivatives.

Preferably, the pharmacologically active ingredient a exhibits psychotropic action, i.e. has a psychotropic effect.

Preferably, the pharmacologically active ingredient a is selected from the group consisting of opiates, opioids, stimulants, tranquilizers, and other narcotics.

In a preferred embodiment, the pharmacologically active ingredient a is an opioid. According to the ATC index, opioids are divided into natural opium alkaloids, phenylpiperidine derivatives, diphenylpropylamine derivatives, benzomorphan derivatives, oripavine derivatives, morphinan derivatives and others.

In another preferred embodiment, the pharmacologically active ingredient a is a stimulant. Stimulants are psychoactive drugs that induce temporary improvements in either mental or physical functions or both. Examples of these kinds of effects may include enhanced wakefulness, locomotion, and alertness. Preferred stimulants are phenylethylamine derivatives. According to the ATC index, stimulants are contained in different classes and groups, e.g. psychoanaleptics, especially psychostimulants, agents used for ADHD and nootropics, particularly centrally acting sympathomimetics; and e.g. nasal preparations, especially nasal decongestants for systemic use, particularly sympathomimetics.

The following opiates, opioids, stimulants, tranquillizers or other narcotics are substances with a psychotropic action, i.e. have a potential of abuse, and hence are preferably contained in the dosage form and the particle(s) A, respectively: alfentanil, allobarbital, allylprodine, alphaprodine, alprazolam, amfepramone, amphetamine, amphetaminil, amobarbital, anileridine, apocodeine, axomadol, barbital, bemidone, benzylmorphine, bezitramide, bromazepam, brotizolam, buprenorphine, butobarbital, butorphanol, camazepam, carfentanil, cathine/D-norpseudoephedrine, cebranopadol, chlordiazepoxide, clobazam clofedanol, clonazepam, clonitazene, clorazepate, clotiazepam, cloxazolam, cocaine, codeine, cyclobarbital, cyclorphan, cyprenorphine, delorazepam, desomorphine, dex-amphetamine, dextromoramide, dextropropoxyphene, dezocine, diampromide, diamorphone, diazepam, dihydrocodeine, dihydromorphine, dihydromorphone, dimenoxadol, dimephetamol, dimethylthiambutene, dioxaphetylbutyrate, dipipanone, dronabinol, eptazocine, estazolam, ethoheptazine, ethylmethylthiambutene, ethyl loflazepate, ethylmorphine, etonitazene, etorphine, faxeladol, fencamfamine, fenethylline, fenpipramide, fenproporex, fentanyl, fludiazepam, flunitrazepam, flurazepam, halazepam, haloxazolam, heroin, hydrocodone, hydromorphone, hydroxypethidine, isomethadone, hydroxymethylmorphinan, ketazolam, ketobemidone, levacetylmethadol (LAAM), levomethadone, levorphanol, levophenacyl-morphane, levoxemacin, lisdexamfetamine dimesylate, lofentanil, loprazolam, lorazepam, lormetazepam, mazindol, medazepam, mefenorex, meperidine, meprobamate, metapon, meptazinol, metazocine, methylmorphine, metamphetamine, methadone, methaqualone, 3-methylfentanyl, 4-methylfentanyl, methylphenidate, methylphenobarbital, methyprylon, metopon, midazolam, modafinil, morphine, myrophine, nabilone, nalbuphene, nalorphine, narceine, nicomorphine, nimetazepam, nitrazepam, nordazepam, norlevorphanol, normethadone, normorphine, norpipanone, opium, oxazepam, oxazolam, oxycodone, oxymorphone, Papaver somniferum, papaveretum, pemoline, pentazocine, pentobarbital, pethidine, phenadoxone, phenomorphane, phenazocine, phenoperidine, piminodine, pholcodeine, phenmetrazine, phenobarbital, phentermine, pinazepam, pipradrol, piritramide, prazepam, profadol, proheptazine, promedol, properidine, propoxyphene, pseudoephedrine, remifentanil, secbutabarbital, secobarbital, sufentanil, tapentadol, temazepam, tetrazepam, tilidine (cis and trans), tramadol, triazolam, vinylbital, N-(1-methyl-2-piperidinoethyl)-N-(2-pyridyl)propionamide, (1R,2R)-3-(3-dimethylamino-1-ethyl-2-methyl-propyl)phenol, (1R,2R,4S)-2-(dimethylamino)methyl-4-(p-fluorobenzyloxy)-1-(m-methoxyphenyl)cyclo-hexanol, (1R,2R)-3-(2-dimethylaminomethyl-cyclohexyl)phenol, (1 S,2S)-3-(3-dimethylamino-1-ethyl-2-methyl-propyl)phenol, (2R,3R)-1-dimethyl amino-3 (3-methoxyphenyl)-2-methyl-pentan-3-01, (1RS,3RS,6RS)-6-dimethylaminomethyl-1-(3-methoxyphenyl)-cyclohexane-1,3-diol, preferably as racemate, 3-(2-dimethylaminomethyl-1-hydroxy-cyclohexyl)phenyl 2-(4-isobutyl-phenyl)propionate, 3-(2-dimethylaminomethyl-1-hydroxy-cyclohexyl)phenyl 2-(6-methoxy-naphthalen-2-yl)propionate, 3-(2-dimethylaminomethyl-cyclohex-1-enyl)-phenyl 2-(4-isobutyl-phenyl)propionate, 3-(2-dimethylaminomethyl-cyclohex-1-enyl)-phenyl 2-(6-methoxy-naphthalen-2-yl)propionate, (RR—SS)-2-acetoxy-4-trifluoromethyl-benzoic acid 3-(2-dimethylaminomethyl-1-hydroxy-cyclohexyl)-phenyl ester, (RR—SS)-2-hydroxy-4-trifluoromethyl-benzoic acid 3-(2-dimethylaminomethyl-1-hydroxy-cyclohexyl)-phenyl ester, (RR—SS)-4-chloro-2-hydroxy-benzoic acid 3-(2-dimethylaminomethyl-1-hydroxy-cyclohexyl)-phenyl ester, (RR—SS)-2-hydroxy-4-methyl-benzoic acid 3-(2-dimethylaminomethyl-1-hydroxy-cyclohexyl)-phenyl ester, (RR—SS)-2-hydroxy-4-methoxy-benzoic acid 3-(2-dimethylaminomethyl-1-hydroxy-cyclohexyl)-phenyl ester, (RR—SS)-2-hydroxy-5-nitro-benzoic acid 3-(2-dimethylaminomethyl-1-hydroxy-cyclohexyl)-phenyl ester, (RR—SS)-2′,4′-difluoro-3-hydroxy-biphenyl-4-carboxylic acid 3-(2-dimethylaminomethyl-1-hydroxy-cyclohexyl)-phenyl ester, and corresponding stereoisomeric compounds, in each case the corresponding derivatives thereof, physiologically acceptable enantiomers, stereoisomers, diastereomers and racemates and the physiologically acceptable derivatives thereof, e.g. ethers, esters or amides, and in each case the physiologically acceptable compounds thereof, in particular the acid or base addition salts thereof and solvates, e.g. hydrochlorides.

In a preferred embodiment, the pharmacologically active ingredient a is selected from the group consisting of DPI-125, M6G (CE-04-410), ADL-5859, CR-665, NRP290 and sebacoyl dinalbuphine ester.

In a preferred embodiment, the pharmacologically active ingredient a is an opioid selected from the group consisting of oxycodone, hydrocodone, oxymorphone, hydromorphone, morphine, tramadol, tapentadol, cebranopadol and the physiologically acceptable salts thereof.

In another preferred embodiment, the pharmacologically active ingredient a is a stimulant selected from the group consisting of amphetamine, dex-amphetamine, dex-methylphenidate, atomoxetine, caffeine, ephedrine, phenylpropanolamine, phenylephrine, fencamphamin, fenozolone, fenetylline, methylenedioxymethamphetamine (MDMA), methylenedioxypyrovalerone (MDPV), prolintane, lisdexamfetamine, mephedrone, meth-amphetamine, methylphenidate, modafinil, nicotine, pemoline, phenylpropanolamine, propylhexedrine, dimethylamylamine, and pseudoephedrine.

The pharmacologically active ingredient a may be present in form of a physiologically acceptable salt, e.g. physiologically acceptable acid addition salt.

Physiologically acceptable acid addition salts comprise the acid addition salt forms which can conveniently be obtained by treating the base form of the active ingredient with appropriate organic and inorganic acids. Active ingredients containing an acidic proton may be converted into their non-toxic metal or amine addition salt forms by treatment with appropriate organic and inorganic bases. The term addition salt also comprises the hydrates and solvent addition forms which the active ingredients are able to form. Examples of such forms are e.g. hydrates, alcoholates and the like.

The pharmacologically active ingredient a is present in the dosage form in a therapeutically effective amount. The amount that constitutes a therapeutically effective amount varies according to the active ingredients being used, the condition being treated, the severity of said condition, the patient being treated, and the frequency of administration.

The content of the pharmacologically active ingredient a in the dosage form is not limited. The dose of the pharmacologically active ingredient a which is adapted for administration preferably is in the range of 0.1 mg to 500 mg, more preferably in the range of 1.0 mg to 400 mg, even more preferably in the range of 5.0 mg to 300 mg, and most preferably in the range of 10 mg to 250 mg. In a preferred embodiment, the total amount of the pharmacologically active ingredient a that is contained in the dosage form is within the range of from 0.01 to 200 mg, more preferably 0.1 to 190 mg, still more preferably 1.0 to 180 mg, yet more preferably 1.5 to 160 mg, most preferably 2.0 to 100 mg and in particular 2.5 to 80 mg.

The skilled person may readily determine an appropriate amount of pharmacologically active ingredient a to include in a dosage form. For instance, in the case of analgesics, the total amount of pharmacologically active ingredient a present in the dosage form is that sufficient to provide analgesia. The total amount of pharmacologically active ingredient a administered to a patient in a dose will vary depending on numerous factors including the nature of the pharmacologically active ingredient a, the weight of the patient, the severity of the pain, the nature of other therapeutic agents being administered etc.

In a preferred embodiment, the pharmacologically active ingredient a is contained in the dosage form in an amount of 2.5±1 mg, 5.0±2.5 mg, 7.5±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5, 140±5 mg, 150±5 mg, 160±5 mg, 170±5 mg, 180±5 mg, 190±5 mg, 200±5 mg, 210±5 mg, 220±5 mg, 230±5 mg, 240±5 mg, 250±5 mg, 260±5 mg, 270±5 mg, 280±5 mg, 290±5 mg, or 300±5 mg. In another preferred embodiment, the pharmacologically active ingredient a is contained in the dosage form in an amount of 2.5±1 mg, 5.0±2.5 mg, 7.5±2.5 mg, 10±2.5 mg, 15±2.5 mg, 20±2.5 mg, 25±2.5 mg, 30±2.5 mg, 35±2.5 mg, 40±2.5 mg, 45±2.5 mg, 50±2.5 mg, 55±2.5 mg, 60±2.5 mg, 65±2.5 mg, 70±2.5 mg, 75±2.5 mg, 80±2.5 mg, 85±2.5 mg, 90±2.5 mg, 95±2.5 mg, 100±2.5 mg, 105±2.5 mg, 110±2.5 mg, 115±2.5 mg, 120±2.5 mg, 125±2.5 mg, 130±2.5 mg, 135±2.5 mg, 140±2.5 mg, 145±2.5 mg, 150±2.5 mg, 155±2.5 mg, 160±2.5 mg, 165±2.5 mg, 170±2.5 mg, 175±2.5 mg, 180±2.5 mg, 185±2.5 mg, 190±2.5 mg, 195±2.5 mg, 200±2.5 mg, 205±2.5 mg, 210±2.5 mg, 215±2.5 mg, 220±2.5 mg, 225±2.5 mg, 230±2.5 mg, 235±2.5 mg, 240±2.5 mg, 245±2.5 mg, 250±2.5 mg, 255±2.5 mg, 260±2.5 mg, or 265±2.5 mg.

In a particularly preferred embodiment, the pharmacologically active ingredient a is tapentadol, preferably its HCl salt, and the dosage form is adapted for administration once daily, twice daily, thrice daily or more frequently. In this embodiment, pharmacologically active ingredient a is preferably contained in the dosage form in an amount of from 25 to 100 mg.

In a particularly preferred embodiment, the pharmacologically active ingredient a is oxymorphone, preferably its HCl salt, and the dosage form is adapted for administration once daily, twice daily, thrice daily or more frequently. In this embodiment, the pharmacologically active ingredient a is preferably contained in the dosage form in an amount of from 5 to 40 mg. In another particularly preferred embodiment, the pharmacologically active ingredient a is oxymorphone, preferably its HCl salt, and the dosage form is adapted for administration once daily. In this embodiment, the pharmacologically active ingredient a is preferably contained in the dosage form in an amount of from 10 to 80 mg.

In another particularly preferred embodiment, the pharmacologically active ingredient a is oxycodone, preferably its HCl salt, and the dosage form is adapted for administration once daily, twice daily, thrice daily or more frequently. In this embodiment, the pharmacologically active ingredient a is preferably contained in the dosage form in an amount of from 5 to 80 mg. Oxycodone, preferably its HCl salt, is preferably combined with acetaminophen as pharmacologically active ingredient b.

In still another particularly preferred embodiment, the pharmacologically active ingredient a is hydromorphone, preferably its HCl, and the dosage form is adapted for administration once daily, twice daily, thrice daily or more frequently. In this embodiment, the pharmacologically active ingredient a is preferably contained in the dosage form in an amount of from 2 to 52 mg. In another particularly preferred embodiment, the pharmacologically active ingredient a is hydromorphone, preferably its HCl, and the dosage form is adapted for administration once daily, twice daily, thrice daily or more frequently. In this embodiment, the pharmacologically active ingredient a is preferably contained in the dosage form in an amount of from 4 to 104 mg.

In yet another particularly preferred embodiment, the pharmacologically active ingredient a is hydrocodone, preferably its bitartrate salt, and the dosage form is adapted for administration once daily, twice daily, thrice daily or more frequently. In this embodiment, the pharmacologically active ingredient a is preferably contained in the dosage form in an amount of from 2.5 to 10 mg. Hydrocodone, preferably its bitartrate salt, is preferably combined with acetamoniphen as pharmacologically active ingredient b.

Preferably, the content of the pharmacologically active ingredient a is at least 0.5 wt.-%, based on the total weight of the dosage form and/or based on the total weight of the particle(s) A.

Preferably, the content of the pharmacologically active ingredient a is at least 2.5 wt.-%, more preferably at least 3.0 wt.-%, still more preferably at least 3.5 wt.-%, yet more preferably at least 4.0 wt.-%, most preferably at least 4.5 wt.-%, based on the total weight of the dosage form and/or based on the total weight of the particle(s) A.

Preferably, the content of the pharmacologically active ingredient a is at most 70 wt.-%, more preferably at most 65 wt.-%, still more preferably at most 60 wt.-%, yet more preferably at most 55 wt.-%, most preferably at most 50 wt.-%, based on the total weight of the dosage form and/or based on the total weight of the particle(s) A.

Preferably, the content of the pharmacologically active ingredient a is within the range of from 0.01 to 80 wt.-%, more preferably 0.1 to 50 wt.-%, still more preferably 1 to 25 wt.-%, based on the total weight of the dosage form and/or based on the total weight of the particle(s) A.

The particle(s) A present in the dosage forms according to the invention preferably comprise 1 to 75 wt.-% of pharmacologically active ingredient a, more preferably 2 to 70 wt.-% of pharmacologically active ingredient a, still more preferably 3 to 65 wt.-% of pharmacologically active ingredient a, based on the total weight of the dosage form and/or based on the total weight of the particle(s) A.

In a preferred embodiment, the content of pharmacologically active ingredient a is within the range of from 0.50±0.45 wt.-%, or 0.75±0.70 wt.-%, or 1.00±0.90 wt.-%, or 1.25±1.20 wt.-%, or 1.50±1.40 wt.-%, or 1.75±1.70 wt.-%, or 2.00±1.90 wt.-%, or 2.25±2.20 wt.-%, or 2.50±2.40 wt.-%, or 2.75±2.50, or 3.00±2.80; more preferably 0.50±0.40 wt.-%, or 0.75±0.60 wt.-%, or 1.00±0.80 wt.-%, or 1.25±1.10 wt.-%, or 1.50±1.25 wt.-%, or 1.75±1.50 wt.-%, or 2.00±1.75 wt.-%, or 2.25±2.00 wt.-%, or 2.50±2.25 wt.-%, or 2.75±2.30, or 3.00±2.60; still more preferably 0.50±0.35 wt.-%, or 0.75±0.50 wt.-%, or 1.00±0.70 wt.-%, or 1.25±1.00 wt.-%, or 1.50±1.15 wt.-%, or 1.75±1.30 wt.-%, or 2.00±1.50 wt.-%, or 2.25±1.90 wt.-%, or 2.50±2.10 wt.-% or 2.75±2.10, or 3.00±2.40; yet more preferably 0.50±0.30 wt.-%, or 0.75±0.40 wt.-%, or 1.00±0.60 wt.-%, or 1.25±0.80 wt.-%, or 1.50±1.00 wt.-%, or 1.75±1.10 wt.-%, or 2.00±1.40 wt.-%, or 2.25±1.60 wt.-%, or 2.50±1.80 wt.-%, or 2.75±1.90, or 3.00±2.20; even more preferably 0.50±0.25 wt.-%, or 0.75±0.30 wt.-%, or 1.00±0.50 wt.-%, or 1.25±0.60 wt.-%, or 1.50±0.80 wt.-%, or 1.75±0.90 wt.-%, or 2.00±1.30 wt.-%, or 2.25±1.40 wt.-%, or 2.50±1.50 wt.-%, or 2.75±1.70, or 3.00±2.00; most preferably 0.50±0.20 wt.-%, or 0.75±0.25 wt.-%, or 1.00±0.40 wt.-%, or 1.25±0.50 wt.-%, or 1.50±0.60 wt.-%, or 1.75±0.70 wt.-%, or 2.00±1.10 wt.-%, or 2.25±1.20 wt.-%, or 2.50±1.30 wt.-% or 2.75±1.50, or 3.00±1.80; and in particular 0.50±0.15 wt.-%, or 0.75±0.20 wt.-%, or 1.00±0.30 wt.-%, or 1.25±0.40 wt.-%, or 1.50±0.50 wt.-%, or 1.75±0.60 wt.-%, or 2.00±0.70 wt.-%, or 2.25±0.80 wt.-%, or 2.50±0.90 wt.-%, or 2.75±1.30, or 3.00±1.60; in each case based on the total weight of the dosage form.

In a preferred embodiment, the content of pharmacologically active ingredient a is within the range of from 2.0±1.9 wt.-%, or 2.5±2.4 wt.-%, or 3.0±2.9 wt.-%, or 3.5±3.4 wt.-%, or 4.0±3.9 wt.-%, or 4.5±4.4 wt.-%, or 5.0±4.9 wt.-%, or 5.5±5.4 wt.-%, or 6.0±5.9 wt.-%; more preferably 2.0±1.7 wt.-%, or 2.5±2.2 wt.-%, or 3.0±2.6 wt.-%, or 3.5±3.1 wt.-%, or 4.0±3.5 wt.-%, or 4.5±4.0 wt.-%, or 5.0±4.4 wt.-%, or 5.5±4.9 wt.-%, or 6.0±5.3 wt.-%, or 6.5±5.8 wt.-%, or 7.0±6.3 wt.-%, or 7.5±6.9 wt.-%, or 8.0±7.4 wt.-%; still more preferably 2.0±1.5 wt.-%, or 2.5±2.0 wt.-%, or 3.0±2.3 wt.-%, or 3.5±2.8 wt.-%, or 4.0±3.1 wt.-%, or 4.5±3.6 wt.-%, or 5.0±3.9 wt.-%, or 5.5±4.4 wt.-%, or 6.0±4.7 wt.-%, or 6.5±5.2 wt.-%, or 7.0±5.8 wt.-%, or 7.5±6.2 wt.-%, or 8.0±6.8 wt.-%; yet more preferably 2.0±1.3 wt.-%, or 2.5±1.8 wt.-%, or 3.0±2.0 wt.-%, or 3.5±2.5 wt.-%, or 4.0±2.7 wt.-%, or 4.5±3.2 wt.-%, or 5.0±3.4 wt.-%, or 5.5±3.9 wt.-%, or 6.0±4.1 wt.-%, or 6.5±4.7 wt.-%, or 7.0±5.2 wt.-%, or 7.5±5.7 wt.-%, or 8.0±6.2 wt.-%; even more preferably 2.0±1.1 wt.-%, or 2.5±1.6 wt.-%, or 3.0±1.7 wt.-%, or 3.5±2.2 wt.-%, or 4.0±2.4 wt.-%, or 4.5±2.8 wt.-%, or 5.0±2.9 wt.-%, or 5.5±3.4 wt.-%, or 6.0±3.5 wt.-%, or 6.5±4.2 wt.-%, or 7.0±4.7 wt.-%, or 7.5±5.2 wt.-%, or 8.0±5.7 wt.-%; most preferably 2.0±0.9 wt.-%, or 2.5±1.4 wt.-%, or 3.0±1.4 wt.-%, or 3.5±1.9 wt.-%, or 4.0±2.1 wt.-%, or 4.5±2.4 wt.-%, or 5.0±2.4 wt.-%, or 5.5±2.9 wt.-%, or 6.0±2.9 wt.-%, or 6.5±3.2 wt.-%, or 7.0±3.7 wt.-%, or 7.5±4.2 wt.-%, or 8.0±4.7 wt.-%; and in particular 2.0±0.7 wt.-%, or 2.5±1.2 wt.-%, or 3.0±1.1 wt.-%, or 3.5±1.6 wt.-%, or 4.0±1.8 wt.-%, or 4.5±2.0 wt.-%, or 5.0±1.9 wt.-%, or 5.5±2.4 wt.-%, or 6.0±2.3 wt.-%, or 6.5±2.7 wt.-%, or 7.0±3.2 wt.-%, or 7.5±3.7 wt.-%, or 8.0±4.2 wt.-%; in each case based on the total weight of the particle(s) A.

In a preferred embodiment, the content of pharmacologically active ingredient a is within the range of from 10±6 wt.-%, more preferably 10±5 wt.-%, still more preferably 10±4 wt.-%, most preferably 10±3 wt.-%, and in particular 10±2 wt.-%, based on the total weight of the dosage form and/or based on the total weight of the particle(s) A. In another preferred embodiment, the content of pharmacologically active ingredient a is within the range of from 15±6 wt.-%, more preferably 15±5 wt.-%, still more preferably 15±4 wt.-%, most preferably 15±3 wt.-%, and in particular 15±2 wt.-%, based on the total weight of the dosage form and/or based on the total weight of the particle(s) A. In still another preferred embodiment, the content of pharmacologically active ingredient a is within the range of from 20±6 wt.-%, more preferably 20±5 wt.-%, still more preferably 20±4 wt.-%, most preferably 20±3 wt.-%, and in particular 20±2 wt.-%, based on the total weight of the dosage form and/or based on the total weight of the particle(s) A. In yet another preferred embodiment, the content of pharmacologically active ingredient a is within the range of from 25±6 wt.-%, more preferably 25±5 wt.-%, still more preferably 25±4 wt.-%, most preferably 25±3 wt.-%, and in particular 25±2 wt.-%, based on the total weight of the dosage form and/or based on the total weight of the particle(s) A. In a further preferred embodiment, the content of pharmacologically active ingredient a is within the range of from 30±6 wt.-%, more preferably 30±5 wt.-%, still more preferably 30±4 wt.-%, most preferably 30±3 wt.-%, and in particular 30±2 wt.-%, based on the total weight of the dosage form and/or based on the total weight of the particle(s) A.

In a preferred embodiment, the content of the pharmacologically active ingredient a is within the range of 35±30 wt.-%, more preferably 35±25 wt.-%, still more preferably 35±20 wt.-%, yet more preferably 35±15 wt.-%, most preferably 35±10 wt.-%, and in particular 35±5 wt.-%, based on the total weight of the dosage form and/or based on the total weight of the particle(s) A. In another preferred embodiment, the content of the pharmacologically active ingredient a is within the range of 45±30 wt.-%, more preferably 45±25 wt.-%, still more preferably 45±20 wt.-%, yet more preferably 45±15 wt.-%, most preferably 45±10 wt.-%, and in particular 45±5 wt.-%, based on the total weight of the dosage form and/or based on the total weight of the particle(s) A. In still another preferred embodiment, the content of the pharmacologically active ingredient a is within the range of 55±30 wt.-%, more preferably 55±25 wt.-%, still more preferably 55±20 wt.-%, yet more preferably 55±15 wt.-%, most preferably 55±10 wt.-%, and in particular 55±5 wt.-%, based on the total weight of the dosage form and/or based on the total weight of the particle(s) A.

The pharmacologically active ingredient a that is included in the preparation of the dosage forms according to the invention preferably has an average particle size of less than 500 microns, still more preferably less than 300 microns, yet more preferably less than 200 or 100 microns. There is no lower limit on the average particle size and it may be, for example, 50 microns. The particle size of pharmacologically active ingredient a (and b) may be determined by any technique conventional in the art, e.g. laser light scattering, sieve analysis, light microscopy or image analysis. Generally speaking it is preferable that the largest dimension of the pharmacologically active ingredient a particle be less than the size of the particle(s) A (e.g. less than the smallest dimension of the particle(s) A).

A skilled person knows how to determine pharmacokinetic parameters such as t_{1/2}, Tₘₐₓ, Cₘₐₓ, AUC and bioavailability. For the purposes of the description, the pharmacokinetic parameters, which may be determined from the blood plasma concentrations of 3-(2-dimethylaminomethylcyclohexyl)phenol, are defined as follows:

| | |
|---|---|
| Cₘₐₓ | maximum measured plasma concentration of the active ingredient |
| | after single administration (≡average peak plasma level) |
| tₘₐₓ | interval of time from administration of the active ingredient until |
| | Cₘₐₓ is reached |
| AUC | total area of the plasma concentration/time curve including the |
| | subarea from the final measured value extrapolated to infinity |
| t_{1/2} | half-life |

The above parameters are in each case stated as mean values of the individual values for all investigated patients/test subjects.

A person skilled in the art knows how the pharmacokinetic parameters of the active ingredient may be calculated from the measured concentrations of the active ingredient in the blood plasma. In this connection, reference may be made, for example, to Willi Cawello (ed.) Parameters for Compartment-free Pharmacokinetics, Shaker Verlag Aachen (1999).

In a preferred embodiment, the pharmacologically active ingredient a is tapentadol or a physiologically acceptable salt thereof, e.g. the hydrochloride. Preferably, the dosage form according to the invention provides a mean absolute bioavailability of tapentadol of at least 22%, more preferably at least 24%, still more preferably at least 26%, yet more preferably at least 28%, most preferably at least 30%, and in particular at least 32%. Tₘₐₓ of tapentadol is preferably within the range of 1.25±1.20 h, more preferably 1.25±1.00 h, still more preferably 1.25±0.80 h, yet more preferably 1.25±0.60 h, most preferably 1.25±0.40 h, and in particular 1.25±0.20 h. t_{1/2} of tapentadol is preferably within the range of 4.0±2.8 h, more preferably 4.0±2.4 h, still more preferably 4.0±2.0 h, yet more preferably 4.0±1.6 h, most preferably 4.0±1.2 h, and in particular 4.0±0.8 h. Preferably, when normalized to a dose of 100 mg tapentadol, Cₘₐₓ of tapentadol is preferably within the range of 90±85 ng/mL, more preferably 90±75 ng/mL, still more preferably 90±65 ng/mL, yet more preferably 90±55 ng/mL, most preferably 90±45 ng/mL, and in particular 90±35 ng/mL; and/or AUC of tapentadol is preferably within the range of 420±400 ng/mL·h, more preferably 420±350 ng/mL·h, still more preferably 420±300 ng/mL·h, yet more preferably 420±250 ng/mL·h, most preferably 420±200 ng/mL·h, and in particular 420±150 ng/mL·h.

In another preferred embodiment, the pharmacologically active ingredient a is oxycodone or a physiologically acceptable salt thereof, e.g. the hydrochloride. Preferably, the dosage form according to the invention provides a mean absolute bioavailability of oxycodone of at least 40%, more preferably at least 45%, still more preferably at least 50%, yet more preferably at least 55%, most preferably at least 60%, and in particular at least 70%. Tₘₐₓ of oxycodone is preferably within the range of 2.6±2.5 h, more preferably 2.6±2.0 h, still more preferably 2.6±1.8 h, yet more preferably 2.6±0.1.6 h, most preferably 2.6±1.4 h, and in particular 2.6±1.2 h. t_{1/2} of oxycodone is preferably within the range of 3.8±3.5 h, more preferably 3.8±3.0 h, still more preferably 3.8±2.5 h, yet more preferably 3.8±2.0 h, most preferably 3.8±1.5 h, and in particular 3.8±1.0 h. Preferably, when normalized to a dose of 30 mg oxycodone, Cₘₐₓ of oxycodone is preferably within the range of 40±35 ng/mL, more preferably 40±30 ng/mL, still more preferably 40±25 ng/mL, yet more preferably 40±20 ng/mL, most preferably 40±15 ng/mL, and in particular 40±10 ng/mL; and/or AUC of oxycodone is preferably within the range of 270±250 ng/mL·h, more preferably 270±200 ng/mL·h, still more preferably 270±150 ng/mL·h, yet more preferably 270±100 ng/mL·h, most preferably 270±75 ng/mL·h, and in particular 270±50 ng/mL·h.

In still another preferred embodiment, the pharmacologically active ingredient a is hydrocodone or a physiologically acceptable salt thereof, e.g. the bitartrate. Tₘₐₓ of hydrocodone is preferably within the range of 1.3±1.2 h, more preferably 1.3±1.0 h, still more preferably 1.3±0.8 h, yet more preferably 1.3±0.6 h, most preferably 1.3±0.4 h, and in particular 1.3±0.2 h. t_{1/2} of hydrocodone is preferably within the range of 3.8±3.5 h, more preferably 3.8±3.0 h, still more preferably 3.8±2.5 h, yet more preferably 3.8±2.0 h, most preferably 3.8±1.5 h, and in particular 3.8±1.0 h.

In yet another preferred embodiment, the pharmacologically active ingredient a is morphine or a physiologically acceptable salt thereof, e.g. the sulfate. Preferably, the dosage form according to the invention provides a mean absolute bioavailability of morphine of at least 15%, more preferably at least 20%, still more preferably at least 25%, yet more preferably at least 30%, most preferably at least 35%, and in particular at least 40%. Tₘₐₓ of morphine is preferably within the range of 0.625±0.60 h, more preferably 0.625±0.50 h, still more preferably 0.625±0.40 h, yet more preferably 0.625±0.30 h, most preferably 0.625±0.20 h, and in particular 0.625±0.15 h. Preferably, when normalized to a dose of 30 mg morphine sulfate, Cₘₐₓ of morphine is preferably within the range of 25±20 ng/mL, more preferably 25±15 ng/mL, still more preferably 25±10 ng/mL, yet more preferably 25±5 ng/mL; and/or AUC of morphine is preferably within the range of 50±45 ng/mL·h, more preferably 50±40 ng/mL·h, still more preferably 50±35 ng/mL·h, yet more preferably 50±30 ng/mL·h, most preferably 50±25 ng/mL·h, and in particular 50±20 ng/mL·h.

In still another preferred embodiment, the pharmacologically active ingredient a is amphetamine or a physiologically acceptable salt thereof. Tₘₐₓ of amphetamine is preferably within the range of 1.7±1.2 h, more preferably 1.7±1.0 h, still more preferably 1.7±0.8 h, yet more preferably 1.7±0.6 h, most preferably 1.7±0.4 h, and in particular 1.7±0.2 h.

In still another preferred embodiment, the pharmacologically active ingredient a is dex-amphetamine or a physiologically acceptable salt thereof, e.g. the sulfate. Tₘₐₓ of dex-amphetamine is preferably within the range of 3.0±2.9 h, more preferably 3.0±2.5 h, still more preferably 3.0±2.1 h, yet more preferably 3.0±1.7 h, most preferably 3.0±1.3 h, and in particular 3.0±0.9 h. t_{1/2} of dex-amphetamine is preferably within the range of 10±6.0 h, more preferably 10±5.0 h, still more preferably 10±4.0 h, yet more preferably 10±3.0 h, most preferably 10±2.0 h, and in particular 10±1.0 h.

The pharmacologically active ingredient b is not particularly limited. The pharmacologically active ingredient b differs from the pharmacologically active ingredient a.

In a preferred embodiment, the pharmacologically active ingredient b exhibits no psychotropic action.

In another preferred embodiment, the pharmacologically active ingredient b is selected from ATC classes [M01A], [M01C], [N02B] and [N02C] according to the WHO.

In a particularly preferred embodiment,
(i) the pharmacologically active ingredient a has a psychotropic effect; and/or
(ii) the pharmacologically active ingredient b is selected from ATC classes [M01A], [M01C], [N02B] and [N02C] according to the WHO.

Preferably, the pharmacologically active ingredient b is selected from the group consisting of acetylsalicylic acid, aloxiprin, choline salicylate, sodium salicylate, salicylamide, salsalate, ethenzamide, morpholine salicylate, dipyrocetyl, benorilate, diflunisal, potassium salicylate, guacetisal, carbasalate calcium, imidazole salicylate, phenazone, metamizole sodium, aminophenazone, propyphenazone, nifenazone, acetaminophen (paracetamol), phenacetin, bucetin, propacetamol, rimazolium, glafenine, floctafenine, viminol, nefopam, flupirtine, ziconotide, methoxyflurane, nabiximols, dihydroergotamine, ergotamine, methysergide, lisuride, flumedroxone, sumatriptan, naratriptan, zolmitriptan, rizatriptan, almotriptan, eletriptan, frovatriptan, pizotifen, clonidine, iprazochrome, dimetotiazine, oxetorone, phenylbutazone, mofebutazone, oxyphenbutazone, clofezone, kebuzone, indomethacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac, bufexamac, piroxicam, tenoxicam, droxicam, lornoxicam, meloxicam, ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, naproxcinod, mefenamic acid, tolfenamic acid, flufenamic acid, meclofenamic acid, celecoxib, rofecoxib, valdecoxib, parecoxib, etoricoxib, lumiracoxib, nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate, oxycinchophen, sodium aurothiomalate, sodium aurotiosulfate, auranofin, aurothioglucose, aurotioprol, penicillamine, bucillamine, their physiologically acceptable salts, as well as mixtures thereof.

In a preferred embodiment, the pharmacologically active ingredient b is acetaminophen or ibuprofen, more preferably acetaminophen.

In a particularly preferred embodiment, the pharmacologically active ingredient a is hydrocodone or a physiologically acceptable salt thereof and the pharmacologically active ingredient b is acetaminophen.

The pharmacologically active ingredient b is present in the dosage form in a therapeutically effective amount. In general, the amount that constitutes a therapeutically effective amount varies according to the pharmacologically active ingredients being used, the condition being treated, the severity of said condition, the patient being treated, and whether the dosage form or the segment in which the pharmacologically active ingredient is contained is designed for an immediate or retarded release.

The total amount of the pharmacologically active ingredient b in the dosage form is not limited. The total amount of the pharmacologically active ingredient b which is adapted for administration preferably is in the range of 0.1 mg to 2,000 mg or 0.1 mg to 1,000 mg or 0.1 mg to 500 mg, more preferably in the range of 1.0 mg to 400 mg, even more preferably in the range of 5.0 mg to 300 mg, and most preferably in the range of 10 mg to 250 mg. In a preferred embodiment, the total amount of the pharmacologically active ingredient b which is contained in the dosage form is within the range of from 10 to 1,000 mg, more preferably 50 to 900 mg, still more preferably 100 to 800 mg, yet more preferably 200 to 600 mg, most preferably 250 to 500 mg and in particular 300 to 400 mg. In another preferred embodiment, the total amount of the pharmacologically active ingredient b which is contained in the dosage form is within the range of from 10 to 500 mg, more preferably 12 to 450 mg, still more preferably 14 to 400 mg, yet more preferably 16 to 375 mg, most preferably 18 to 350 mg and in particular 20 to 325 mg.

In a preferred embodiment, the pharmacologically active ingredient b is contained in the dosage form in an amount of 7.5±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5, 140±5 mg, 150±5 mg, 160±5 mg, 170±5 mg, 180±5 mg, 190±5 mg, 200±5 mg, 210±5 mg, 220±5 mg, 230±5 mg, 240±5 mg, or 250±5 mg. In another preferred embodiment, the pharmacologically active ingredient b is contained in the dosage form in an amount of 5±2.5 mg, 7.5±2.5 mg, 10±2.5 mg, 15±2.5 mg, 20±2.5 mg, 25±2.5 mg, 30±2.5 mg, 35±2.5 mg, 40±2.5 mg, 45±2.5 mg, 50±2.5 mg, 55±2.5 mg, 60±2.5 mg, 65±2.5 mg, 70±2.5 mg, 75±2.5 mg, 80±2.5 mg, 85±2.5 mg, 90±2.5 mg, 95±2.5 mg, 100±2.5 mg, 105±2.5 mg, 110±2.5 mg, 115±2.5 mg, 120±2.5 mg, 125±2.5 mg, 130±2.5 mg, 135±2.5 mg, 140±2.5 mg, 145±2.5 mg, 150±2.5 mg, 155±2.5 mg, 160±2.5 mg, 165±2.5 mg, 170±2.5 mg, 175±2.5 mg, 180±2.5 mg, 185±2.5 mg, 190±2.5 mg, 195±2.5 mg, 200±2.5 mg, 205±2.5 mg, 210±2.5 mg, 215±2.5 mg, 220±2.5 mg, 225±2.5 mg, 230±2.5 mg, 235±2.5 mg, 240±2.5 mg, 245±2.5 mg, or 250±2.5 mg. In still another preferred embodiment, the pharmacologically active ingredient b is contained in the dosage form in an amount of 250±10 mg, 275±10 mg, 300±10 mg, 325±10 mg, 350±10 mg, 375±10 mg, 400±10 mg, 425±10 mg, 450±10 mg, 475±10 mg, 500±10 mg, 525±10 mg, 550±10 mg, 575±10 mg or 600±10 mg.

The total content of the pharmacologically active ingredient b preferably ranges from about 0.01 wt.-% to about 95 wt.-%, more preferably from about 0.1 wt.-% to about 80 wt.-%, even more preferably from about 1.0 wt.-% to about 50 wt.-%, yet more preferably from about 1.5 wt.-% to about 30 wt.-%, and most preferably from about 2.0 wt.-% to 20 wt.-%, based on the total weight of the dosage form.

Preferably, the total content of the pharmacologically active ingredient b is within the range of from 0.01 to 80 wt.-%, more preferably 0.1 to 50 wt.-%, still more preferably 1 to 25 wt.-%, based on the total weight of the dosage form. In a preferred embodiment, the total content of the pharmacologically active ingredient b is within the range of from 20±15 wt.-%, more preferably 20±12 wt.-%, still more preferably 20±10 wt.-%, most preferably 20±7 wt.-%, and in particular 20±5 wt.-%, based on the total weight of the dosage form. In a preferred embodiment, the total content of the pharmacologically active ingredient b is within the range of from 30±15 wt.-%, more preferably 30±12 wt.-%, still more preferably 30±10 wt.-%, most preferably 30±7 wt.-%, and in particular 30±5 wt.-%, based on the total weight of the dosage form. In a preferred embodiment, the total content of the pharmacologically active ingredient b is within the range of from 40±15 wt.-%, more preferably 40±12 wt.-%, still more preferably 40±10 wt.-%, most preferably 40±7 wt.-%, and in particular 40±5 wt.-%, based on the total weight of the dosage form. In a preferred embodiment, the total content of the pharmacologically active ingredient b is within the range of from 50±15 wt.-%, more preferably 50±12 wt.-%, still more preferably 50±10 wt.-%, most preferably 50±7 wt.-%, and in particular 50±5 wt.-%, based on the total weight of the dosage form. In a preferred embodiment, the total content of the pharmacologically active ingredient b is within the range of from 60±15 wt.-%, more preferably 60±12 wt.-%, still more preferably 60±10 wt.-%, most preferably 60±7 wt.-%, and in particular 60±5 wt.-%, based on the total weight of the dosage form.

In a particularly preferred embodiment, the pharmacologically active ingredient b is acetaminophen. In this embodiment, the acetaminophen is preferably contained in the particle(s) B or the dosage form in an amount of from 100 to 600 mg, more preferably 150 to 550 mg, still more preferably 200 to 500 mg, most preferably 250 to 450 mg and in particular 275 to 400 mg.

In another particularly preferred embodiment, the pharmacologically active ingredient b is ibuprofen. In this embodiment, the ibuprofen is preferably contained in the particle(s) B or the dosage form in an amount of from 100 to 600 mg, more preferably 150 to 550 mg, still more preferably 200 to 500 mg, most preferably 250 to 450 mg and in particular 275 to 400 mg.

The pharmacologically active ingredient b that is included in the preparation of the dosage forms according to the invention preferably has an average particle size of less than 500 microns, still more preferably less than 300 microns, yet more preferably less than 200 or 100 microns. There is no lower limit on the average particle size and it may be, for example, 50 microns. Generally speaking it is preferable that the largest dimension of the pharmacologically active ingredient b particle be less than the size of the particle(s) A (e.g. less than the smallest dimension of the particle(s) A).

Preferred combinations A¹ to A³⁶ of the pharmacologically active ingredient a and the pharmacologically active ingredient b are summarized in the table here below, wherein the pharmacologically active ingredient a as well as the pharmacologically active ingredient b each also refer to the physiologically acceptable salts thereof, particularly to the hydrochlorides or bitartrates:

| | a | b | | a | b |
|---|---|---|---|---|---|
| A¹ | oxycodone | ibuprofen | A¹⁰ | oxycodone | acetaminophen |
| A² | oxymorphone | ibuprofen | A¹¹ | oxymorphone | acetaminophen |
| A³ | hydrocodone | ibuprofen | A¹² | hydrocodone | acetaminophen |
| A⁴ | hydromorphone | ibuprofen | A¹³ | hydromorphone | acetaminophen |
| A⁵ | morphine | ibuprofen | A¹⁴ | morphine | acetaminophen |
| A⁶ | tapentadol | ibuprofen | A¹⁵ | tapentadol | acetaminophen |
| A⁷ | tramadol | ibuprofen | A¹⁶ | tramadol | acetaminophen |
| A⁸ | buprenorphine | ibuprofen | A¹⁷ | buprenorphine | acetaminophen |
| A⁹ | pseudoephedrine | ibuprofen | A¹⁸ | pseudoephedrine | acetaminophen |
| A¹⁹ | oxycodone | diclofenac | A²⁸ | oxycodone | acetylsalicylic acid |
| A²⁰ | oxymorphone | diclofenac | A²⁹ | oxymorphone | acetylsalicylic acid |
| A²¹ | hydrocodone | diclofenac | A³⁰ | hydrocodone | acetylsalicylic acid |
| A²² | hydromorphone | diclofenac | A³¹ | hydromorphone | acetylsalicylic acid |
| A²³ | morphine | diclofenac | A³² | morphine | acetylsalicylic acid |
| A²⁴ | tapentadol | diclofenac | A³³ | tapentadol | acetylsalicylic acid |
| A²⁵ | tramadol | diclofenac | A³⁴ | tramadol | acetylsalicylic acid |
| A²⁶ | buprenorphine | diclofenac | A³⁵ | buprenorphine | acetylsalicylic acid |
| A²⁷ | pseudoephedrine | diclofenac | A³⁶ | pseudoephedrine | acetylsalicylic acid |

In a preferred embodiment, the relative weight ratio of the total content of the pharmacologically active ingredient a to the total content of the pharmacologically active ingredient b [a:b] is within the range of (8±1):1, more preferably (7±1):1, still more preferably (6±1):1, yet more preferably (5±1):1, even more preferably (4±1):1, most preferably (3±1):1 and in particular (2±1):1.

In still another preferred embodiment, the relative weight ratio of the total content of the pharmacologically active ingredient b to the total content of the pharmacologically active ingredient a [b:a] is within the range of (8±1):1, more preferably (7±1):1, still more preferably (6±1):1, yet more preferably (5±1):1, even more preferably (4±1):1, most preferably (3±1):1 and in particular (2±1):1.

Preferably, the relative weight ratio of the total content of the pharmacologically active ingredient b to the total content of the pharmacologically active ingredient a [b:a] is within the range of from 10:1 to 150:1, more preferably 10:1 to 50:1, or 30:1 to 140:1.

The dosage form according to the invention provides fast release, preferably immediate release under in vitro conditions of the pharmacologically active ingredient a, and independently of the pharmacologically active ingredient b in accordance with Ph. Eur.

Unless expressed otherwise all percent are in wt.-%.

Preferably, the dosage form according to the invention provides an release profile such that under in vitro conditions (i) in 600 ml 0.1 M HCl (pH 1) at 75 rpm, or (ii) in 900 ml demineralized water at 50 rpm, after 30 min (USP apparatus II) at least 50 wt.-%, preferably at least 80 wt.-% of the pharmacologically active ingredient a that was originally contained in the dosage form as well as independently at least 50 wt.-%, preferably at least 80 wt.-% of the pharmacologically active ingredient b that was originally contained in the dosage form, have been released.

The term “immediate release” as applied to dosage forms is understood by persons skilled in the art which has structural implications for the respective dosage forms. The term is defined, for example, in the current issue of the US Pharmacopoeia (USP), General Chapter 1092, “THE DISSOLUTION PROCEDURE: DEVELOPMENT AND VALIDATION”, heading “STUDY DESIGN”, “Time Points”. For immediate-release dosage forms, the duration of the procedure is typically 30 to 60 minutes; in most cases, a single time point specification is adequate for Pharmacopeia purposes. Industrial and regulatory concepts of product comparability and performance may require additional time points, which may also be required for product registration or approval. A sufficient number of time points should be selected to adequately characterize the ascending and plateau phases of the dissolution curve. According to the Biopharmaceutics Classification System referred to in several FDA Guidances, highly soluble, highly permeable drugs formulated with rapidly dissolving products need not be subjected to a profile comparison if they can be shown to release 85% or more of the active drug substance within 15 minutes. For these types of products a one-point test will suffice. However, most products do not fall into this category. Dissolution profiles of immediate-release products typically show a gradual increase reaching 85% to 100% at 30 to 45 minutes. Thus, dissolution time points in the range of 15, 20, 30, 45, and 60 minutes are usual for most immediate-release products.

In a particularly preferred embodiment, under in vitro conditions (i) in 600 ml 0.1 M HCl (pH 1) at 75 rpm, or (ii) in 900 ml demineralized water at 50 rpm, using the basket method according to Ph. Eur. at 75 rpm, after 1 h under in vitro conditions the dosage form has released at least 60% more preferably at least 65%, still more preferably at least 70%, yet more preferably at least 75%, even more preferably at least 80%, most preferably at least 85% and in particular at least 90% or at least 95% or at least 99% of the pharmacologically active ingredient a that was originally contained in the dosage form, and independently at least 60% more preferably at least 65%, still more preferably at least 70%, yet more preferably at least 75%, even more preferably at least 80%, most preferably at least 85% and in particular at least 90% or at least 95% or at least 99% of the pharmacologically active ingredient b that was originally contained in the dosage form.

Preferably, under in vitro conditions the dosage form according to the invention has released after 30 minutes at least 70%, more preferably at least 75%, still more preferably at least 80%, yet more preferably at least 82%, most preferably at least 84% and in particular at least 86% of the pharmacologically active ingredient a originally contained in the dosage form, and independently at least 70%, more preferably at least 75%, still more preferably at least 80%, yet more preferably at least 82%, most preferably at least 84% and in particular at least 86% of the pharmacologically active ingredient b originally contained in the dosage form.

Preferably, under in vitro conditions the dosage form according to the invention has released after 10 minutes at least 70%, more preferably at least 73%, still more preferably at least 76%, yet more preferably at least 78%, most preferably at least 80% and in particular at least 82% of the pharmacologically active ingredient a originally contained in the dosage form, and independently at least 70%, more preferably at least 73%, still more preferably at least 76%, yet more preferably at least 78%, most preferably at least 80% and in particular at least 82% of the pharmacologically active ingredient b originally contained in the dosage form.

Preferably, under in vitro conditions the dosage form has released after 5 minutes at least 10%, after 10 minutes at least 20%, after 15 minutes at least 30%, after 20 minutes at least 40%, after 30 minutes at least 60%, after 40 minutes at least 70%, after 50 minutes at least 80%, after 60 minutes at least 90% or 99% of the pharmacologically active ingredient a that was originally contained in the dosage form, and independently after 5 minutes at least 10%, after 10 minutes at least 20%, after 15 minutes at least 30%, after 20 minutes at least 40%, after 30 minutes at least 60%, after 40 minutes at least 70%, after 50 minutes at least 80%, after 60 minutes at least 90% or 99% of the pharmacologically active ingredient b that was originally contained in the dosage form.

Preferably, the dosage form releases in 600 ml 0.1 M HCl, pH 1 and at 75 rpm using an USP apparatus II at least 50 wt.-% of the pharmacologically active ingredient a originally contained in the dosage form; and/or at least 50 wt.-% of the pharmacologically active ingredient b originally contained in the dosage form.

Suitable in vitro conditions are known to the skilled artisan. In this regard it can be referred to, e.g., the Eur. Ph. Preferably, the release profile is measured under the following conditions: Paddle apparatus equipped without sinker, 50 rpm, 37±5° C., 600 mL simulated intestinal fluid pH 6.8 (phosphate buffer) or pH 4.5. In a preferred embodiment, the rotational speed of the paddle is increased to 75 rpm. In another preferred embodiment, the release profile is determined under the following conditions: basket method, 75 rpm, 37±5° C., 600 mL 0.1 N HCl or 600 mL of SIF sp (pH 6.8) or 600 mL of 0.1 N HCl+40% ethanol.

Further preferred release profiles B¹ to B¹⁰ that independently apply to the release of pharmacologically active ingredient a and pharmacologically active ingredient b are summarized in the table here below [all data in wt.-% of released pharmacologically active ingredient a/b]:

| time | B¹ | B² | B³ | B⁴ | B⁵ | B⁶ | B⁷ | B⁸ | B⁹ | B¹⁰ |
|---|---|---|---|---|---|---|---|---|---|---|
| 10 min | ≧30 | ≧35 | ≧40 | ≧45 | ≧50 | ≧60 | ≧70 | ≧80 | ≧80 | ≧80 |
| 20 min | ≧50 | ≧55 | ≧60 | ≧65 | ≧70 | ≧75 | ≧80 | ≧85 | ≧90 | ≧95 |
| 30 min | ≧55 | ≧60 | ≧65 | ≧70 | ≧75 | ≧85 | ≧90 | ≧95 | ≧95 | ≧95 |
| 40 min | ≧60 | ≧65 | ≧70 | ≧80 | ≧85 | ≧90 | ≧95 | ≧95 | ≧95 | ≧95 |
| 50 min | ≧65 | ≧70 | ≧80 | ≧85 | ≧88 | ≧92 | ≧95 | ≧95 | ≧95 | ≧95 |
| 60 min | ≧75 | ≧80 | ≧85 | ≧90 | ≧92 | ≧94 | ≧95 | ≧95 | ≧95 | ≧95 |

Preferably, the release profile, the pharmaceutically active ingredients a/b and the pharmaceutical excipients of the dosage form according to the invention are stable upon storage, preferably upon storage at elevated temperature, e.g. 40° C., for 3 months in sealed containers.

In connection with the release profile “stable” means that when comparing the initial release profile with the release profile after storage, at any given time point the release profiles deviate from one another by not more than 20%, more preferably not more than 15%, still more preferably not more than 10%, yet more preferably not more than 7.5%, most preferably not more than 5.0% and in particular not more than 2.5%.

In connection with the drug and the pharmaceutical excipients “stable” means that the dosage forms satisfy the requirements of EMEA concerning shelf-life of pharmaceutical products.

The dosage form according to the invention may comprise more than a single pharmacologically active ingredient a and/or more than a single pharmacologically active ingredient a.

The dosage form according to the invention may also comprise one or more additional pharmacologically active ingredient(s) c. The additional pharmacologically active ingredient c may be susceptible to abuse or not. Additional pharmacologically active ingredient(s) c may be present within the particle(s) A or outside the particle(s) A.

While in a preferred embodiment the dosage form according to the invention does not contain an opioid antagonist, in another preferred embodiment the dosage form according to the invention, preferably the particle(s) A, comprise an opioid (agonist) as well as an opioid antagonist.

Any conventional opioid antagonist may be present, e.g. naltrexone or naloxone or their pharmaceutically acceptable salts. Naloxone, including its salts, is particularly preferred. The opioid antagonist may be present within the particle(s) A or within the matrix. Alternatively, opioid antagonist may be provided in separate particle(s) A to the pharmacologically active ingredient a. The preferred composition of such particle(s) A is the same as that described for pharmacologically active ingredient a-containing particle(s) A.

The ratio of opioid agonist to opioid antagonist in the dosage forms according to the invention is preferably 1:1 to 3:1 by weight, for example, 2:1 by weight.

In another preferred embodiment, neither the particle(s) A nor the dosage form comprise any opioid antagonist.

In a preferred embodiment, the dosage form according to the invention is adapted for administration once daily. In another preferred embodiment, the dosage form according to the invention is adapted for administration twice daily. In still another preferred embodiment, the dosage form according to the invention is adapted for administration thrice daily. In yet another preferred embodiment, the dosage form according to the invention is adapted for administration more frequently than thrice daily, for example 4 times daily, 5 times daily, 6 times daily, 7 times daily or 8 times daily.

For the purpose of the specification, “twice daily” means equal or nearly equal time intervals, i.e., every 12 hours, or different time intervals, e.g., 8 and 16 hours or 10 and 14 hours, between the individual administrations.

For the purpose of the specification, “thrice daily” means equal or nearly equal time intervals, i.e., every 8 hours, or different time intervals, e.g., 6, 6 and 12 hours; or 7, 7 and 10 hours, between the individual administrations.

Preferably, the dosage form according to the invention has under in vitro conditions a disintegration time measured in accordance with Ph. Eur. of at most 10 minutes, more preferably at most 8 minutes, or at most 6 minutes, or at most 5 minutes, more preferably at most 4 minutes, still more preferably at most 3 minutes, yet more preferably at most 2.5 minutes, most preferably at most 2 minutes and in particular at most 1.5 minutes.

It has been surprisingly found that oral dosage forms can be designed that provide the best compromise between tamper-resistance, disintegration time and drug release, drug load, processability (especially tablettability) and patient compliance.

Tamper-resistance and drug release antagonize each other. While smaller particle(s) A should typically show a faster release of the pharmacologically active ingredient a, tamper-resistance requires some minimal size of the particle(s) A in order to effectively prevent abuse, e.g. i.v. administration. The larger the particle(s) A are the less they are suitable for being abused nasally. The smaller the particle(s) A are the faster gel formation occurs. Thus, drug release on the one hand and tamper-resistance on the other hand can be optimized by finding the best compromise.

The dosage form according to the invention comprises one or more particle(s) A, typically a multitude of particles A. The particle(s) A comprise a pharmacologically active ingredient a, which is embedded in a polymer matrix that preferably comprises a polyalkylene oxide and preferably further excipients.

For the purpose of the specification, the term “particle” refers to a discrete mass of material that is solid, e.g. at 20° C. or at room temperature or ambient temperature. Preferably a particle is solid at 20° C. Preferably, the individual particle(s) A are monoliths. The multitude of particles A, however, is not monolithic, but multiparticulate. Preferably, the pharmacologically active ingredient a and the constituents of the polymer matrix are intimately homogeneously distributed in the particle(s) A so that the particle(s) A do not contain any segments where either pharmacologically active ingredient a is present in the absence of polymer matrix or where polymer matrix is present in the absence of pharmacologically active ingredient a.

It is principally possible that the dosage form according to the invention comprises a single particle A.

In another preferred embodiment, the dosage form according to the invention comprises a plurality of particles A, more preferably a multitude of particles A.

In a preferred embodiment, the dosage form comprises at least 2, or at least 3, or at least 4, or at least 5 particles A. Preferably, the dosage form comprises not more than 10, or not more than 9, or not more than 8, or not more than 7 particles A.

In another preferred embodiment, the particles A amount to a total number within the range of from 20 to 600. More preferably, the dosage form comprises at least 30, or at least 60, or at least 90, or at least 120, or at least 150 particles A. Preferably, the dosage form comprises not more than 500, or not more than 400, or not more than 300, or not more than 200 particles A.

Preferably, when the dosage form contains more than a single particle A, the individual particles A may be of the same or of different size, shape and/or composition.

In a preferred embodiment, all particles A are made from the same mixture of ingredients and/or are substantially of the same size, shape, weight and composition.

In another preferred embodiment, particles A can be divided into at least 2 or at least 3 different types, e.g. particles A₁, particles A₂, and optionally particles A₃, that differ from one another in at least one property, preferably being selected from the group consisting of size, shape, weight, composition, release profile, breaking strength and resistance against solvent extraction.

The content of the particle(s) is not particularly limited and preferably amounts to a total content within the range of from 10 wt.-% to 80 wt.-%, based on the total weight of the dosage form. Preferably, the content of the particle(s) A in the dosage forms according to the invention is at most 99 wt.-%, or at most 98 wt.-%, or at most 96 wt.-%, or at most 94 wt.-%, more preferably at most 92 wt.-%, or at most 90 wt.-%, or at most 88 wt.-%, or at most 86 wt.-%, still more preferably at most 84 wt.-%, or at most 82 wt.-%, or at most 80 wt.-%, or at most 78 wt.-%, yet more preferably at most 76 wt.-%, or at most 74 wt.-%, or at most 72 wt.-%, or at most 70 wt.-%, most preferably at most 65 wt.-%, or at most 60 wt.-%, or at most 55 wt.-%, or at most 50 wt.-%, and in particular at most 45 wt.-%, or at most 40 wt.-%, or at most 35 wt.-%, or at most 30 wt.-%, based on the total weight of the dosage form.

Preferably, the content of the particle(s) A in the dosage forms according to the invention is at least 2.5 wt.-%, at least 3.0 wt.-%, at least 3.5 wt.-% or at least 4.0 wt.-%; more preferably at least 4.5 wt.-%, at least 5.0 wt.-%, at least 5.5 wt.-% or at least 6.0 wt.-%; still more preferably at least 6.5 wt.-%, at least 7.0 wt.-%, at least 7.5 wt.-% or at least 8.0 wt.-%; yet more preferably at least 8.5 wt.-%, at least 9.0 wt.-%, at least 9.5 wt.-% or at least 10 wt.-%; even more preferably at least 11 wt.-%, at least 12 wt.-%, at least 13 wt.-% or at least 14 wt.-%; most preferably at least 15 wt.-%, at least 17.5 wt.-%, at least 20 wt.-% or at least 22.5 wt.-%; and in particular at least 25 wt.-%, at least 27.5 wt.-%, at least 30 wt.-% or at least 35 wt.-%; based on the total weight of the dosage form.

In a preferred embodiment, the content of the particle(s) A in the dosage forms according to the invention is within the range of 10±7.5 wt.-%, more preferably 10±5.0 wt.-%, still more preferably 10±4.0 wt.-%, yet more preferably 10±3.0 wt.-%, most preferably 10±2.0 wt.-%, and in particular 10±1.0 wt.-%, based on the total weight of the dosage form. In another preferred embodiment, the content of the particle(s) A in the dosage forms according to the invention is within the range of 15±12.5 wt.-%, more preferably 15±10 wt.-%, still more preferably 15±8.0 wt.-%, yet more preferably 15±6.0 wt.-%, most preferably 15±4.0 wt.-%, and in particular 15±2.0 wt.-%, based on the total weight of the dosage form. In still another preferred embodiment, the content of the particle(s) A in the dosage forms according to the invention is within the range of 20±17.5 wt.-%, more preferably 20±15 wt.-%, still more preferably 20±12.5 wt.-%, yet more preferably 20±10 wt.-%, most preferably 20±7.5 wt.-%, and in particular 20±5 wt.-%, based on the total weight of the dosage form. In yet another preferred embodiment, the content of the particle(s) A in the dosage forms according to the invention is within the range of 25±17.5 wt.-%, more preferably 25±15 wt.-%, still more preferably 25±12.5 wt.-%, yet more preferably 25±10 wt.-%, most preferably 25±7.5 wt.-%, and in particular 25±5 wt.-%, based on the total weight of the dosage form. In another preferred embodiment, the content of the particle(s) A in the dosage forms according to the invention is within the range of 30±17.5 wt.-%, more preferably 30±15 wt.-%, still more preferably 30±12.5 wt.-%, yet more preferably 30±10 wt.-%, most preferably 30±7.5 wt.-%, and in particular 30±5 wt.-%, based on the total weight of the dosage form. In still another preferred embodiment, the content of the particle(s) A in the dosage forms according to the invention is within the range of 35±17.5 wt.-%, more preferably 35±15 wt.-%, still more preferably 35±12.5 wt.-%, yet more preferably 35±10 wt.-%, most preferably 35±7.5 wt.-%, and in particular 35±5 wt.-%, based on the total weight of the dosage form. In another preferred embodiment, the content of the particle(s) A in the dosage forms according to the invention is within the range of 40±17.5 wt.-%, more preferably 40±15 wt.-%, still more preferably 40±12.5 wt.-%, yet more preferably 40±10 wt.-%, most preferably 40±7.5 wt.-%, and in particular 40±5 wt.-%, based on the total weight of the dosage form.

In a preferred embodiment, the dosage form according to the invention comprises one or more particle(s) A comprising a pharmacologically active ingredient a as well as one or more particle(s) B comprising a pharmacologically active ingredient b. As besides the different pharmacologically active ingredient a and b, respectively, the particle(s) A and the particle(s) B have preferably, but independently of one another corresponding composition and properties, in the following it is referred to “particle(s)” meaning that these preferred embodiments independently apply to particle(s) A as well as to optionally present particle(s) B.

When the particle(s) are film coated, the polymer matrix is preferably homogeneously distributed in the core of the dosage form, i.e. the film coating preferably does not contain polymer matrix. Nonetheless, the film coating as such may of course contain one or more polymers, which however, preferably differ from the constituents of the polymer matrix contained in the core.

The shape of the particle(s) is not particularly limited. As the particle(s) are preferably manufactured by hot-melt extrusion, preferred particle(s) present in the dosage forms according to the invention are generally cylindrical in shape. The diameter of such particle(s) is therefore the diameter of their circular cross section. The cylindrical shape is caused by the extrusion process according to which the diameter of the circular cross section is a function of the extrusion die and the length of the cylinders is a function of the cutting length according to which the extruded strand of material is cut into pieces of preferably more or less predetermined length.

The suitability of cylindrical, i.e. a spherical particle(s) for the manufacture of the dosage forms according to the invention is unexpected. Typically, the aspect ratio is regarded as an important measure of the spherical shape. The aspect ratio is defined as the ratio of the maximal diameter (dₘₐₓ) and its orthogonal Feret-diameter. For aspherical particle(s), the aspect ratio has values above 1. The smaller the value the more spherical is the particle(s). Aspect ratios below 1.1 are typically considered satisfactory, aspect ratios above 1.2, however, are typically considered not suitable for the manufacture of conventional dosage forms. The inventors have surprisingly found that when manufacturing the dosage forms according to the invention, even particle(s) having aspect ratios above 1.2 can be processed without difficulties and that it is not necessary to provide spherical particle(s). In a preferred embodiment, the aspect ratio of the particle(s) is at most 1.40, more preferably at most 1.35, still more preferably at most 1.30, yet more preferably at most 1.25, even more preferably at most 1.20, most preferably at most 1.15 and in particular at most 1.10. In another preferred embodiment, the aspect ratio of the particle(s) is at least 1.10, more preferably at least 1.15, still more preferably at least 1.20, yet more preferably at least 1.25, even more preferably at least 1.30, most preferably at least 1.35 and in particular at least 1.40.

The particle(s) are of macroscopic size, typically the average diameter is within the range of from 100 μm to 1500 μm, preferably 200 μm to 1500 μm, more preferably 300 μm to 1500 μm, still more preferably 400 μm to 1500 μm, most preferably 500 μm to 1500 μm, and in particular 600 μm to 1500 μm.

The particle(s) in the dosage forms according to the invention are of macroscopic size, i.e. typically have an average particle(s) size of at least 50 μm, more preferably at least 100 μm, still more preferably at least 150 μm or at least 200 μm, yet more preferably at least 250 μm or at least 300 μm, most preferably at least 400 μm or at least 500 μm, and in particular at least 550 μm or at least 600 μm.

Preferred particle(s) have an average length and average diameter of 1000 μm or less. When the particle(s) are manufactured by extrusion technology, the “length” of particle(s) is the dimension of the particle(s) that is parallel to the direction of extrusion. The “diameter” of particle(s) is the largest dimension that is perpendicular to the direction of extrusion.

Particularly preferred particle(s) have an average diameter of less than 1000 μm, more preferably less than 800 still more preferably of less than 650 μm. Especially preferred particle(s) have an average diameter of less than 700 μm, particularly less than 600 μm, still more particularly less than 500 μm, e.g. less than 400 μm. Particularly preferred particle(s) have an average diameter in the range 200 to 1000 μm, more preferably 400 to 800 μm, still more preferably 450 to 700 μm, yet more preferably 500 to 650 μm, e.g. 500 to 600 μm. Further preferred particle(s) have an average diameter of between 300 μm and 400 μm, of between 400 μm and 500 μm, or of between 500 μm and 600 μm, or of between 600 μm and 700 μm or of between 700 μm and 800 μm.

Preferred particle(s) that are present in the dosage forms according to the invention have an average length of less than 1000 μm, preferably an average length of less than 800 μm, still more preferably an average length of less than 650 μm, e.g. a length of 800 μm, 700 μm 600 μm, 500 μm, 400 μm or 300 μm. Especially preferred particle(s) have an average length of less than 700 μm, particularly less than 650 μm, still more particularly less than 550 μm, e.g. less than 450 μm. Particularly preferred particle(s) therefore have an average length in the range 200-1000 μm, more preferably 400-800 μm, still more preferably 450-700 μm, yet more preferably 500-650 μm, e.g. 500-600 μm. The minimum average length of the microparticle(s) is determined by the cutting step and may be, e.g. 500 μm, 400 μm, 300 μm or 200 μm.

In a preferred embodiment, the particle(s) have (i) an average diameter of 1000±300 μm, more preferably 1000±250 μm, still more preferably 1000±200 μm, yet more preferably 1000±150 μm, most preferably 1000±100 μm, and in particular 1000±50 μm; and/or (ii) an average length of 1000±300 μm, more preferably 1000±250 μm, still more preferably 1000±200 μm, yet more preferably 1000±150 μm, most preferably 1000±100 μm, and in particular 1000±50 μm.

The size of particle(s) may be determined by any conventional procedure known in the art, e.g. laser light scattering, sieve analysis, light microscopy or image analysis.

Preferably, the individual particle(s) have a weight within the range of from 0.1 mg to 5.0 mg.

In preferred embodiments, the individual particle(s) preferably have a weight within the range of 1.0±0.9 mg, or 1.0±0.8 mg, or 1.0±0.7 mg, or 1.0±0.6 mg, or 1.0±0.5 mg, or 1.0±0.4 mg, or 1.0±0.3 mg; or 1.5±0.9 mg, or 1.5±0.8 mg, or 1.5±0.7 mg, or 1.5±0.6 mg, or 1.5±0.5 mg, or 1.5±0.4 mg, or 1.5±0.3 mg; or 2.0±0.9 mg, or 2.0±0.8 mg, or 2.0±0.7 mg, or 2.0±0.6 mg, or 2.0±0.5 mg, or 2.0±0.4 mg, or 2.0±0.3 mg; or 2.5±0.9 mg, or 2.5±0.8 mg, or 2.5±0.7 mg, or 2.5±0.6 mg, or 2.5±0.5 mg, or 2.5±0.4 mg, or 2.5±0.3 mg; or 3.0±0.9 mg, or 3.0±0.8 mg, or 3.0±0.7 mg, or 3.0±0.6 mg, or 3.0±0.5 mg, or 3.0±0.4 mg, or 3.0±0.3 mg.

Preferably, the particle(s) A have a total weight over all particles A within the range of from 10 mg to 500 mg. In preferred embodiments, the total weight of the particle(s) A is within the range of 180±170 mg, or 180±150 mg, or 180±130 mg, or 180±110 mg, or 180±90 mg, or 180±70 mg, or 180±50 mg, or 180±30 mg.

Preferably, the particle(s) that are contained in the dosage form according to the invention have an arithmetic average weight, in the following referred to as “aaw”, wherein at least 70%, more preferably at least 75%, still more preferably at least 80%, yet more preferably at least 85%, most preferably at least 90% and in particular at least 95% of the individual particle(s) contained in said one or more particle(s) has an individual weight within the range of aaw±30%, more preferably aaw±25%, still more preferably aaw±20%, yet more preferably aaw±15%, most preferably aaw±10%, and in particular aaw±5%. For example, if the dosage form according to the invention contains a plurality of 100 particles and aaw of said plurality of particles is 1.00 mg, at least 75 individual particles (i.e. 75%) have an individual weight within the range of from 0.70 to 1.30 mg (1.00 mg±30%).

In a preferred embodiment, the particle(s) are not film coated. In another preferred embodiment, the particle(s) are film coated.

The particle(s) according to the invention can optionally be provided, partially or completely, with a conventional coating. The particle(s) according to the invention are preferably film coated with conventional film coating compositions. Suitable coating materials are commercially available, e.g. under the trademarks Opadry® and Eudragit®.

When the particle(s) are film coated, the content of the dried film coating is preferably at most 5 wt.-%, more preferably at most 4 wt.-%, still more preferably at most 3.5 wt.-%, yet more preferably at most 3 wt.-%, most preferably at most 2.5 wt.-%, and in particular at most 2 wt.-%, based on the total weight of the particle(s). In a particularly preferred embodiment, the weight increase based on the total weight of the dosage form and/or based on the total weight of the particle(s) (uncoated starting material) is within the range of from 3.0 to 4.7 wt.-%, more preferably 3.1 to 4.6 wt.-%, still more preferably 3.2 to 4.5 wt.-%, yet more preferably 3.3 to 4.4 wt.-%, most preferably 3.4 to 4.3 wt.-%, and in particular 3.5 to 4.2 wt.-%.

In a preferred embodiment of the invention, the film coating of the particle(s) A contains the total amount of the pharmacologically active ingredient b or a portion b_{C} thereof.

The tamper-resistant dosage form according to the invention comprises one or more particle(s) A which comprise a polymer matrix, wherein the polymer matrix preferably comprises a polyalkylene oxide, preferably at a content of at least 25 wt.-%, based on the total weight of the dosage form and/or based on the total weight of the particle(s) A. The optionally present particle(s) B may also, independently of the particle(s) A, comprise a polymer matrix, wherein the polymer matrix preferably comprises a polyalkylene oxide, preferably at a content of at least 25 wt.-%, based on the total weight of the dosage form and/or based on the total weight of the particle(s) B.

Preferably, the polyalkylene oxide is selected from polymethylene oxide, polyethylene oxide and polypropylene oxide, or copolymers thereof. Polyethylene oxide is preferred.

Preferably, the polyalkylene oxide has a weight average molecular weight of at least 200,000 g/mol, more preferably at least 500,000 g/mol. In a preferred embodiment, the polyalkylene oxide has a weight average molecular weight (M_{W}) or viscosity average molecular weight (M_{η}) of at least 750,000 g/mol, preferably at least 1,000,000 g/mol or at least 2,500,000 g/mol, more preferably in the range of 1,000,000 g/mol to 15,000,000 g/mol, and most preferably in the range of 5,000,000 g/mol to 10,000,000 g/mol. Suitable methods to determine M_{W} and M_{η} are known to a person skilled in the art. M_{η} is preferably determined by rheological measurements, whereas M_{W} can be determined by gel permeation chromatography (GPC).

Polyalkylene oxide may comprise a single polyalkylene oxide having a particular average molecular weight, or a mixture (blend) of different polymers, such as two, three, four or five polymers, e.g., polymers of the same chemical nature but different average molecular weight, polymers of different chemical nature but same average molecular weight, or polymers of different chemical nature as well as different molecular weight.

For the purpose of the specification, a polyalkylene glycol has a molecular weight of up to 20,000 g/mol whereas a polyalkylene oxide has a molecular weight of more than 20,000 g/mol. In a preferred embodiment, the weight average over all molecular weights of all polyalkylene oxides that are contained in the dosage form is at least 200,000 g/mol. Thus, polyalkylene glycols, if any, are preferably not taken into consideration when determining the weight average molecular weight of polyalkylene oxide.

The polyalkylene oxide may be combined with one or more different polymers selected from the group consisting of polyalkylene oxide, preferably polymethylene oxide, polyethylene oxide, polypropylene oxide; polyethylene, polypropylene, polyvinyl chloride, polycarbonate, polystyrene, polyvinylpyrrolidone, poly(alk)acrylate, poly(hydroxy fatty acids), such as for example poly(3-hydroxybutyrate-co-3-hydroxyvalerate) (Biopol®), poly(hydroxyvaleric acid); polycaprolactone, polyvinyl alcohol, polyesteramide, polyethylene succinate, polylactone, polyglycolide, polyurethane, polyamide, polylactide, polyacetal (for example polysaccharides optionally with modified side chains), polylactide/glycolide, polylactone, polyglycolide, polyorthoester, polyanhydride, block polymers of polyethylene glycol and polybutylene terephthalate (Polyactive®), polyanhydride (Polifeprosan), copolymers thereof, block-copolymers thereof (e.g., Poloxamer®), and mixtures of at least two of the stated polymers, or other polymers with the above characteristics.

Preferably, the molecular weight dispersity M_{w}/Mₙ of polyalkylene oxide is within the range of 2.5±2.0, more preferably 2.5±1.5, still more preferably 2.5±1.0, yet more preferably 2.5±0.8, most preferably 2.5±0.6, and in particular 2.5±0.4.

The polyalkylene oxide preferably has a viscosity at 25° C. of 30 to 17,600 cP, more preferably 55 to 17,600 cP, still more preferably 600 to 17,600 cP and most preferably 4,500 to 17,600 cP, measured in a 5 wt.-% aqueous solution using a model RVF Brookfield viscosimeter (spindle no. 2/rotational speed 2 rpm); of 400 to 4,000 cP, more preferably 400 to 800 cP or 2,000 to 4,000 cP, measured on a 2 wt.-% aqueous solution using the stated viscosimeter (spindle no. 1 or 3/rotational speed 10 rpm); or of 1,650 to 10,000 cP, more preferably 1,650 to 5,500 cP, 5,500 to 7,500 cP or 7,500 to 10,000 cP, measured on a 1 wt.-% aqueous solution using the stated viscosimeter (spindle no. 2/rotational speed 2 rpm).

Polyethylene oxide that is suitable for use in the dosage forms according to the invention is commercially available from Dow. For example, Polyox WSR N-12K, Polyox N-60K, Polyox WSR 301 NF or Polyox WSR 303NF may be used in the dosage forms according to the invention. For details concerning the properties of these products, it can be referred to e.g. the product specification.

Preferably, the overall content of the polyalkylene oxide is within the range of from 25 to 80 wt.-%, more preferably 25 to 75 wt.-%, still more preferably 25 to 70 wt.-%, yet more preferably 25 to 65 wt.-%, most preferably 30 to 65 wt.-% and in particular 35 to 65 wt.-%, based on the total weight of the dosage form and/or based on the total weight of the particle(s). In a preferred embodiment, the content of the polyalkylene oxide is at least 30 wt.-%, more preferably at least 35 wt.-%, still more preferably at least 40 wt.-%, yet more preferably at least 45 wt.-% and in particular at least 50 wt.-%, based on the total weight of the dosage form and/or based on the total weight of the particle(s).

In a preferred embodiment, the overall content of polyalkylene oxide is within the range of 35±8 wt.-%, more preferably 35±6 wt.-%, most preferably 35±4 wt.-%, and in particular 35±2 wt.-%, based on the total weight of the dosage form and/or based on the total weight of the particle(s). In another preferred embodiment, the overall content of polyalkylene oxide is within the range of 40±12 wt.-%, more preferably 40±10 wt.-%, most preferably 40±7 wt.-%, and in particular 40±3 wt.-%, based on the total weight of the dosage form and/or based on the total weight of the particle(s). In still another preferred embodiment, the overall content of polyalkylene oxide is within the range of 45±16 wt.-%, more preferably 45±12 wt.-%, most preferably 45±8 wt.-%, and in particular 45±4 wt.-%, based on the total weight of the dosage form and/or based on the total weight of the particle(s). In yet another preferred embodiment, the overall content of polyalkylene oxide is within the range of 50±20 wt.-%, more preferably 50±15 wt.-%, most preferably 50±10 wt.-%, and in particular 50±5 wt.-%, based on the total weight of the dosage form and/or based on the total weight of the particle(s). In a further preferred embodiment, the overall content of polyalkylene oxide is within the range of 55±20 wt.-%, more preferably 55±15 wt.-%, most preferably 55±10 wt.-%, and in particular 55±5 wt.-%, based on the total weight of the dosage form and/or based on the total weight of the particle(s). In still a further a preferred embodiment, the overall content of polyalkylene oxide is within the range of 60±20 wt.-%, more preferably 60±15 wt.-%, most preferably 60±10 wt.-%, and in particular 60±5 wt.-%. In a still further a preferred embodiment, the overall content of polyalkylene oxide is within the range of 65±20 wt.-%, more preferably 65±15 wt.-%, and most preferably 65±10 wt.-%, and in particular 65±5 wt.-%, based on the total weight of the dosage form and/or based on the total weight of the particle(s).

Preferably, the relative weight ratio of the polyalkylene oxide to the pharmacologically active ingredient a is within the range of 30:1 to 1:10, more preferably 20:1 to 1:1, still more preferably 15:1 to 5:1, yet more preferably 14:1 to 6:1, most preferably 13:1 to 7:1, and in particular 12:1 to 8:1.

The dosage form according to the invention is tamper-resistant.

As used herein, the term “tamper-resistant” refers to dosage forms that are preferably resistant to conversion into a form suitable for misuse or abuse, particular for nasal and/or intravenous administration, by conventional means such as grinding in a mortar or crushing by means of a hammer. In this regard, the dosage forms as such may be crushable by conventional means. However, the particle(s) A contained in the dosage forms according to the invention preferably exhibit mechanical properties such that they cannot be pulverized by conventional means any further. The same may independently apply to the optionally present particle(s) B. As the particle(s) A are of macroscopic size and contain the pharmacologically active ingredient a, and as the optionally present particle(s) B may independently be of macroscopic size and contain the pharmacologically active ingredient b, they cannot be administered nasally thereby rendering the dosage forms tamper-resistant.

Preferably, the particle(s) A have a breaking strength of at least 300 N. Preferably, the overall dosage form as such does not have a breaking strength of at least 300 N, i.e. typically the breaking strength of the dosage form as such, e.g. of the tablet or capsule, is below 300 N.

When the dosage form additionally contains particle(s) B, these particle(s) B may also have a breaking strength of at least 300 N. However, though being less preferred, the invention also includes embodiments where optionally present particle(s) B do not have a breaking strength of at least 300 N.

Preferably, the particle(s) are tamper-resistant as such so that they also provide tamper-resistance after they have been separated from the remaining constituents of the dosage form. Thus, preferably the particle(s) as such contain all ingredients that are necessary to render them tamper-resistant.

Preferably, when trying to tamper the dosage form in order to prepare a formulation suitable for abuse by intravenous administration, the liquid part of the formulation that can be separated from the remainder by means of a syringe is as less as possible, preferably it contains not more than 20 wt.-%, more preferably not more than 15 wt.-%, still more preferably not more than 10 wt.-%, and most preferably not more than 5 wt.-% of the originally contained pharmacologically active ingredient a.

The same may apply to pharmacologically active ingredient b. However, in a preferred embodiment pharmacologically active ingredient a is more prone to abuse than pharmacologically active ingredient b.

Preferably, this property is tested by (i) dispensing a dosage form that is either intact or has been manually comminuted by means of two spoons in 5 ml of purified water, (ii) heating the liquid up to its boiling point, (iii) boiling the liquid in a covered vessel for 5 min without the addition of further purified water, (iv) drawing up the hot liquid into a syringe (needle 21 G equipped with a cigarette filter), (v) determining the amount of the pharmacologically active ingredient a and/or b contained in the liquid within the syringe.

Further, when trying to disrupt the dosage forms by means of a hammer or mortar, the particle(s) preferably tend to adhere to one another thereby forming aggregates and agglomerates, respectively, which are larger in size than the untreated particle(s).

Preferably, tamper-resistance is achieved based on the mechanical properties of the particle(s) so that comminution is avoided or at least substantially impeded. According to the invention, the term comminution means the pulverization of the particle(s) using conventional means usually available to an abuser, for example a pestle and mortar, a hammer, a mallet or other conventional means for pulverizing under the action of force. Thus, tamper-resistance preferably means that pulverization of the particle(s) using conventional means is avoided or at least substantially impeded.

Preferably, the mechanical properties of the particle(s) according to the invention, particularly their breaking strength and deformability, substantially rely on the presence and spatial distribution of a polymer matrix, preferably comprising polyalkylene oxide, although its mere presence does typically not suffice in order to achieve said properties. The advantageous mechanical properties of the particle(s) according to the invention may not automatically be achieved by simply processing pharmacologically active ingredient a/b, the components of the polymer matrix such as polyalkylene oxide, and optionally further excipients by means of conventional methods for the preparation of dosage forms. In fact, usually suitable apparatuses must be selected for the preparation and critical processing parameters must be adjusted, particularly pressure/force, temperature and time. Thus, even if conventional apparatuses are used, the process protocols usually must be adapted in order to meet the required criteria.

In general, the particle(s) exhibiting the desired properties may be obtained only if, during preparation of the particle(s),
suitable components
in suitable amounts

are exposed to
a sufficient pressure
at a sufficient temperature
for a sufficient period of time.

Thus, regardless of the apparatus used, the process protocols must be adapted in order to meet the required criteria. Therefore, the breaking strength and deformability of the particle(s) is separable from the composition.

The particle(s) contained in the dosage form according to the invention preferably have a breaking strength of at least 300 N, at least 400 N, or at least 500 N, preferably at least 600 N, more preferably at least 700 N, still more preferably at least 800 N, yet more preferably at least 1000 N, most preferably at least 1250 N and in particular at least 1500 N.

In order to verify whether a particle(s) exhibits a particular breaking strength of e.g. 300 N or 500 N it is typically not necessary to subject said particle(s) to forces much higher than 300 N and 500 N, respectively. Thus, the breaking strength test can usually be terminated once the force corresponding to the desired breaking strength has been slightly exceeded, e.g. at forces of e.g. 330 N and 550 N, respectively.

The “breaking strength” (resistance to crushing) of a dosage form and of a particle(s) is known to the skilled person. In this regard it can be referred to, e.g., W. A. Ritschel, Die Tablette, 2. Auflage, Editio Cantor Verlag Aulendorf, 2002; H Liebermann et al., Dosage forms: Dosage forms, Vol. 2, Informa Healthcare; 2 edition, 1990; and Encyclopedia of Pharmaceutical Technology, Informa Healthcare; 1 edition.

For the purpose of the specification, the breaking strength is preferably defined as the amount of force that is necessary in order to fracture the particle(s) (=breaking force). Therefore, for the purpose of the specification a particle does preferably not exhibit the desired breaking strength when it breaks, i.e., is fractured into at least two independent parts that are separated from one another.

In another preferred embodiment, however, the particle is regarded as being broken if the force decreases by 50% (threshold value) of the highest force measured during the measurement (see below).

The particle(s) according to the invention are distinguished from conventional particles that can be contained in dosage forms in that, due to their breaking strength, they cannot be pulverized by the application of force with conventional means, such as for example a pestle and mortar, a hammer, a mallet or other usual means for pulverization, in particular devices developed for this purpose (tablet crushers). In this regard “pulverization” means crumbling into small particles. Avoidance of pulverization virtually rules out oral or parenteral, in particular intravenous or nasal abuse.

Conventional particles typically have a breaking strength well below 200 N.

The breaking strength of conventional round dosage forms/particle may be estimated according to the following empirical formula: Breaking Strength [in N]=10×Diameter Of The Dosage form/Particle [in mm]. Thus, according to said empirical formula, a round dosage form/particle having a breaking strength of at least 300 N would require a diameter of at least 30 mm. Such a particles, however, could not be swallowed, let alone a dosage form containing a plurality of such particles. The above empirical formula preferably does not apply to the particle(s) according to the invention, which are not conventional but rather special.

Further, the actual mean chewing force is 220 N (cf., e.g., P. A. Proeschel et al., J Dent Res, 2002, 81(7), 464-468). This means that conventional particles having a breaking strength well below 200 N may be crushed upon spontaneous chewing, whereas the particle(s) according to the invention may preferably not.

Still further, when applying a gravitational acceleration of 9.81 m/s², 300 N correspond to a gravitational force of more than 30 kg, i.e. the particle(s) according to the invention can preferably withstand a weight of more than 30 kg without being pulverized.

Methods for measuring the breaking strength of a dosage form are known to the skilled artisan. Suitable devices are commercially available.

For example, the breaking strength (resistance to crushing) can be measured in accordance with the Eur. Ph. 5.0, 2.9.8 or 6.0, 2.09.08 “Resistance to Crushing of Dosage forms”. The test is intended to determine, under defined conditions, the resistance to crushing of dosage forms and of the particle(s), respectively, measured by the force needed to disrupt them by crushing. The apparatus consists of 2 jaws facing each other, one of which moves towards the other. The flat surfaces of the jaws are perpendicular to the direction of movement. The crushing surfaces of the jaws are flat and larger than the zone of contact with the dosage form and a single particle, respectively. The apparatus is calibrated using a system with a precision of 1 Newton. The dosage form and particle, respectively, is placed between the jaws, taking into account, where applicable, the shape, the break-mark and the inscription; for each measurement the dosage form and particle, respectively, is oriented in the same way with respect to the direction of application of the force (and the direction of extension in which the breaking strength is to be measured). The measurement is carried out on 10 dosage forms and particles, respectively, taking care that all fragments have been removed before each determination. The result is expressed as the mean, minimum and maximum values of the forces measured, all expressed in Newton.

A similar description of the breaking strength (breaking force) can be found in the USP. The breaking strength can alternatively be measured in accordance with the method described therein where it is stated that the breaking strength is the force required to cause a dosage form and particle, respectively, to fail (i.e., break) in a specific plane. The dosage forms and particle, respectively, are generally placed between two platens, one of which moves to apply sufficient force to the dosage form and particle, respectively, to cause fracture. For conventional, round (circular cross-section) dosage forms and particles, respectively, loading occurs across their diameter (sometimes referred to as diametral loading), and fracture occurs in the plane. The breaking force of a dosage form and a particle, respectively, is commonly called hardness in the pharmaceutical literature; however, the use of this term is misleading. In material science, the term hardness refers to the resistance of a surface to penetration or indentation by a small probe. The term crushing strength is also frequently used to describe the resistance of dosage forms and particles, respectively, to the application of a compressive load. Although this term describes the true nature of the test more accurately than does hardness, it implies that dosage forms and particles, respectively, are actually crushed during the test, which is often not the case.

Alternatively, the breaking strength (resistance to crushing) can be measured in accordance with WO 2008/107149, which can be regarded as a modification of the method described in the Eur. Ph. The apparatus used for the measurement is preferably a “Zwick Z 2.5” materials tester, Fₘₐₓ=2.5 kN with a maximum draw of 1150 mm, which should be set up with one column and one spindle, a clearance behind of 100 mm and a test speed adjustable between 0.1 and 800 mm/min together with testControl software. A skilled person knows how to properly adjust the test speed, e.g. to 10 mm/min, 20 mm/min, or 40 mm/min, for example. Measurement is performed using a pressure piston with screw-in inserts and a cylinder (diameter 10 mm), a force transducer, Fₘₐₓ. 1 kN, diameter=8 mm, class 0.5 from 10 N, class 1 from 2 N to ISO 7500-1, with manufacturer's test certificate M according to DIN 55350-18 (Zwick gross force Fₘₐₓ=1.45 kN) (all apparatus from Zwick GmbH & Co. KG, Ulm, Germany) with Order No BTC-FR 2.5 TH. D09 for the tester, Order No BTC-LC 0050N. P01 for the force transducer, Order No BO 70000 S06 for the centring device.

When using the testControl software (testXpert V10.11), the following exemplified settings and parameters have revealed to be useful: LE-position: clamping length 150 mm. LE-speed: 500 mm/min, clamping length after pre-travel: 195 mm, pre-travel speed: 500 mm/min, no pre-force control—pre-force: pre-force 1N, pre-force speed 10 mm/min—sample data: no sample form, measuring length traverse distance 10 mm, no input required prior to testing—testing/end of test; test speed: position-controlled 10 mm/min, delay speed shift: 1, force shut down threshold 50% Fₘₐₓ, no force threshold for break-tests, no max length variation, upper force limit: 600N—expansion compensation: no correction of measuring length—actions after testing: LE to be set after test, no unload of sample—TRS: data memory: TRS distance interval until break 1 μm, TRS time interval 0.1 s, TRS force interval 1N—machine; traverse distance controller: upper soft end 358 mm, lower soft end 192 mm—lower test space. Parallel arrangement of the upper plate and the ambos should be ensured—these parts must not touch during or after testing. After testing, a small gap (e.g. 0.1 or 0.2 mm) should still be present between the two brackets in intimated contact with the tested particle, representing the remaining thickness of the deformed particle.

In a preferred embodiment, the particle is regarded as being broken if it is fractured into at least two separate pieces of comparable morphology. Separated matter having a morphology different from that of the deformed particle, e.g. dust, is not considered as pieces qualifying for the definition of breaking.

The particle(s) according to the invention preferably exhibit mechanical strength over a wide temperature range, in addition to the breaking strength (resistance to crushing) optionally also sufficient hardness, yield strength, fatigue strength, impact resistance, impact elasticity, tensile strength, compressive strength and/or modulus of elasticity, optionally also at low temperatures (e.g. below −24° C., below −40° C. or possibly even in liquid nitrogen), for it to be virtually impossible to pulverize by spontaneous chewing, grinding in a mortar, pounding, etc. Thus, preferably, the comparatively high breaking strength of the particle(s) according to the invention is maintained even at low or very low temperatures, e.g., when the dosage form is initially chilled to increase its brittleness, for example to temperatures below −25° C., below −40° C. or even in liquid nitrogen.

The particle(s) according to the invention are preferably characterized by a certain degree of breaking strength. This does not mean that the particle(s) must also exhibit a certain degree of hardness. Hardness and breaking strength are different physical properties. Therefore, the tamper-resistance of the dosage form does not necessarily depend on the hardness of the particle(s). For instance, due to their breaking strength, impact strength, elasticity modulus and tensile strength, respectively, the particle(s) can preferably be deformed, e.g. plastically, when exerting an external force, for example using a hammer, but cannot be pulverized, i.e., crumbled into a high number of fragments. In other words, the particle(s) according to the invention are preferably characterized by a certain degree of breaking strength, but not necessarily also by a certain degree of form stability.

Therefore, in the meaning of the specification, a particle that is deformed when being exposed to a force in a particular direction of extension but that does not break (plastic deformation or plastic flow) is preferably to be regarded as having the desired breaking strength in said direction of extension.

Preferred particle(s) present in the dosage forms according to the invention are those having a suitable tensile strength as determined by a test method currently accepted in the art. Further preferred particle(s) are those having a Youngs Modulus as determined by a test method of the art. Still further preferred particle(s) are those having an acceptable elongation at break.

Irrespective of whether the particle(s) according to the invention have an increased breaking strength or nor, the particle(s) according to the invention preferably exhibit a certain degree of deformability. The particle(s) contained in the dosage form according to the invention preferably have a deformability such that they show an increase, preferably a substantially steady increase of the force at a corresponding decrease of the displacement in the force-displacement-diagram when being subjected to a breaking strength test as described above.

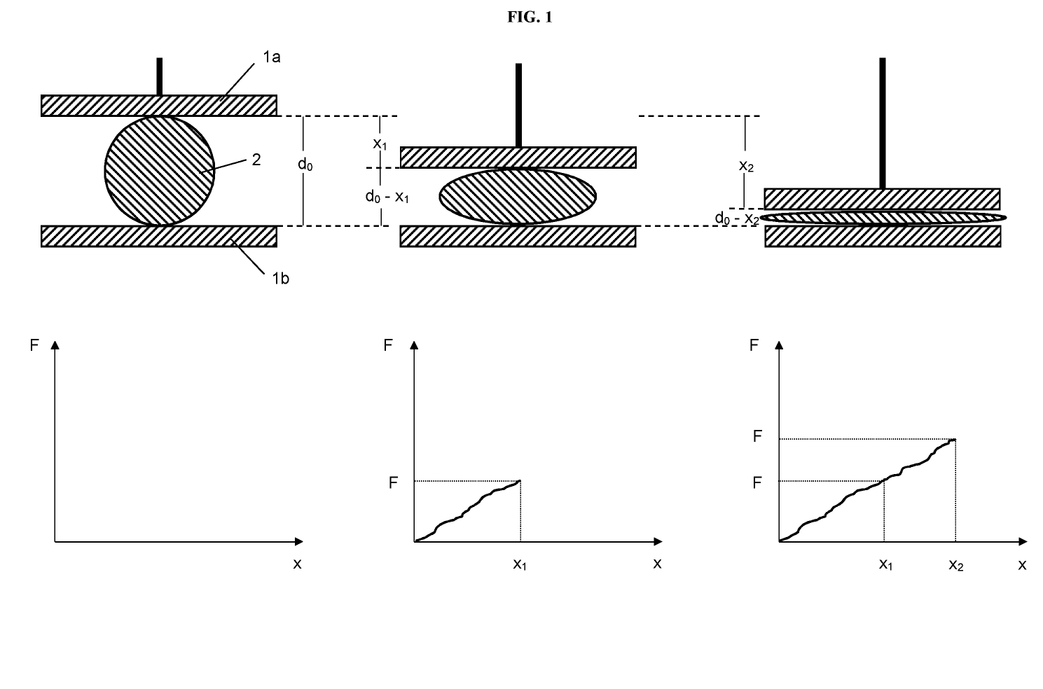

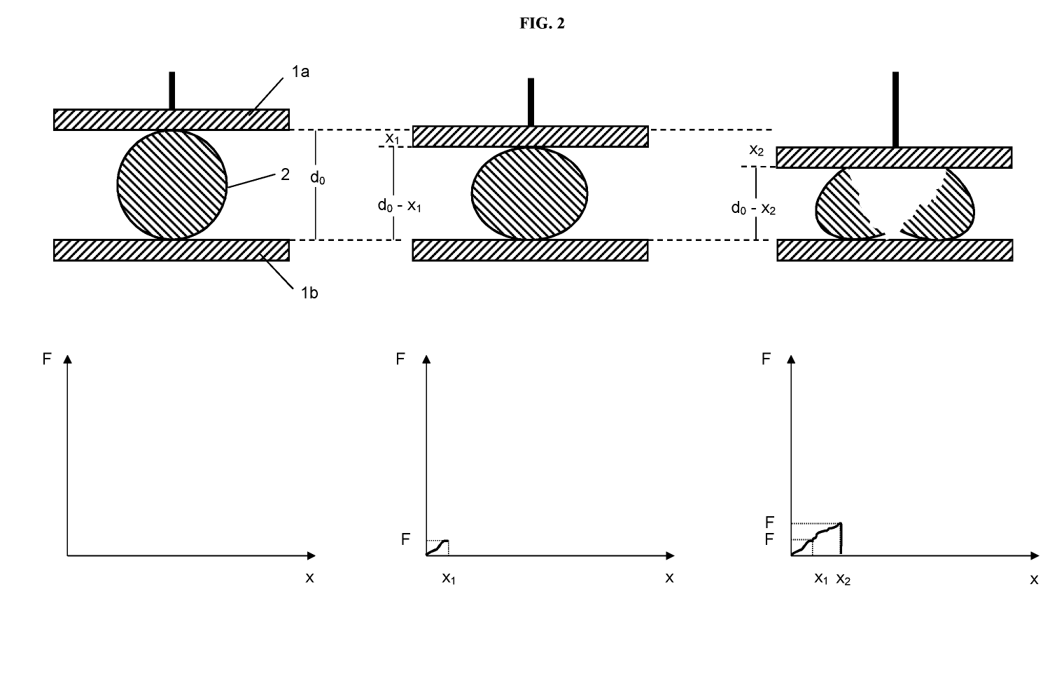

This mechanical property, i.e. the deformability of the individual particle(s), is illustrated in FIGS. 1 and 2.

FIG. 1 schematically illustrates the measurement and the corresponding force-displacement-diagram. In particular, FIG. 1A shows the initial situation at the beginning of the measurement. The sample particle (2) is placed between upper jaw (1a) and lower jaw (1b) which each are in intimate contact with the surface of the particle (2). The initial displacement d₀ between upper jaw (1a) and lower jaw (1b) corresponds to the extension of the particle orthogonal to the surfaces of upper jaw (1a) and lower jaw (1b). At this time, no force is exerted at all and thus, no graph is displayed in the force-displacement-diagram below. When the measurement is commenced, the upper jaw is moved in direction of lower jaw (1b), preferably at a constant speed. FIG. 1B shows a situation where due to the movement of upper jaw (1a) towards lower jaw (1b) a force is exerted on particle (2). Because of its deformability, the particle (2) is flattened without being fractured. The force-displacement-diagram indicates that after a reduction of the displacement d₀ of upper jaw (1a) and lower jaw (1b) by distance x₁, i.e. at a displacement of d₁=d₀−x₁, a force F₁ is measured. FIG. 1C shows a situation where due to the continuous movement of upper jaw (1a) towards lower jaw (1b), the force that is exerted on particle (2) causes further deformation, although the particle (2) does not fracture. The force-displacement-diagram indicates that after a reduction of the displacement d₀ of upper jaw (1a) and lower jaw (1b) by distance x₂, i.e. at a displacement of d₂=d₀−x₂, a force F₂ is measured. Under these circumstances, the particle (2) has not been broken (fractured) and a substantially steady increase of the force in the force-displacement-diagram is measured.

In contrast, FIG. 2 schematically illustrates the measurement and the corresponding force-displacement-diagram of a conventional comparative particle not having the degree of deformability as the particle(s) according to the invention. FIG. 2A shows the initial situation at the beginning of the measurement. The comparative sample particle (2) is placed between upper jaw (l a) and lower jaw (1b) which each are in intimate contact with the surface of the comparative particle (2). The initial displacement d₀ between upper jaw (1a) and lower jaw (1b) corresponds to the extension of the comparative particle orthogonal to the surfaces of upper jaw (1a) and lower jaw (1b). At this time, no force is exerted at all and thus, no graph is displayed in the force-displacement-diagram below. When the measurement is commenced, the upper jaw is moved in direction of lower jaw (1b), preferably at a constant speed. FIG. 2B shows a situation where due to the movement of upper jaw (1a) towards lower jaw (1b) a force is exerted on comparative particle (2). Because of some deformability, the comparative particle (2) is slightly flattened without being fractured. The force-displacement-diagram indicates that after a reduction of the displacement d₀ of upper jaw (1a) and lower jaw (1b) by distance x₁, i.e. at a displacement of d₁=d₀−x₁, a force F₁ is measured. FIG. 2C shows a situation where due to the continuous movement of upper jaw (l a) towards lower jaw (1b), the force that is exerted on particle (2) causes sudden fracture of the comparative particle (2). The force-displacement-diagram indicates that after a reduction of the displacement d₀ of upper jaw (1a) and lower jaw (1b) by distance x₂, i.e. at a displacement of d₂=d₀−x₂, a force F₂ is measured that suddenly drops when the particle fractures. Under these circumstances, the particle (2) has been broken (fractured) and no steady increase of the force in the force-displacement-diagram is measured. The sudden drop (decrease) of the force can easily be recognized and does not need to be quantified for the measurement. The steady increase in the force-displacement-diagram ends at displacement d₂=d₀−x₂ when the particle breaks.

In a preferred embodiment, the particle(s) contained in the dosage form according to the invention have a deformability such that they show an increase, preferably a substantially steady increase of the force at a corresponding decrease of the displacement in the force-displacement-diagram when being subjected to a breaking strength test as described above (“Zwick Z 2.5” materials tester, constant speed), preferably at least until the displacement d of upper jaw (1a) and lower jaw (1b) has been reduced to a value of 90% of the original displacement d₀ (i.e. d=0.9·d₀), preferably to a displacement d of 80% of the original displacement d₀, more preferably to a displacement d of 70% of the original displacement d₀, still more preferably to a displacement d of 60% of the original displacement d₀, yet more preferably to a displacement d of 50% of the original displacement d₀, even more preferably to a displacement d of 40% of the original displacement d₀, most preferably to a displacement d of 30% of the original displacement d₀, and in particular to a displacement d of 20% of the original displacement d₀, or to a displacement d of 15% of the original displacement d₀, to a displacement d of 10% of the original displacement d₀, or to a displacement d of 5% of the original displacement d₀.

In another preferred embodiment, the particle(s) contained in the dosage form according to the invention have a deformability such that they show an increase, preferably a substantially steady increase of the force at a corresponding decrease of the displacement in the force-displacement-diagram when being subjected to a breaking strength test as described above (“Zwick Z 2.5” materials tester, constant speed), preferably at least until the displacement d of upper jaw (1a) and lower jaw (1b) has been reduced to 0.80 mm or 0.75 mm, preferably 0.70 mm or 0.65 mm, more preferably 0.60 mm or 0.55 mm, still more preferably 0.50 mm or 0.45 mm, yet more preferably 0.40 mm or 0.35 mm, even more preferably 0.30 mm or 0.25 mm, most preferably 0.20 mm or 0.15 mm and in particular 0.10 or 0.05 mm.

In still another preferred embodiment, the particle(s) contained in the dosage form according to the invention have a deformability such that they show an increase, preferably a substantially steady increase of the force at a corresponding decrease of the displacement in the force-displacement-diagram when being subjected to a breaking strength test as described above (“Zwick Z 2.5” materials tester, constant speed), at least until the displacement d of upper jaw (1a) and lower jaw (1b) has been reduced to 50% of the original displacement d₀ (i.e. d=d₀/2), whereas the force measured at said displacement (d=d₀/2) is at least 25 N or at least 50 N, preferably at least 75 N or at least 100 N, still more preferably at least 150 N or at least 200 N, yet more preferably at least 250 N or at least 300 N, even more preferably at least 350 N or at least 400 N, most preferably at least 450 N or at least 500 N, and in particular at least 625 N, or at least 750 N, or at least 875 N, or at least 1000 N, or at least 1250 N, or at least 1500 N.

In another preferred embodiment, the particle(s) contained in the dosage form according to the invention have a deformability such that they show an increase, preferably a substantially steady increase of the force at a corresponding decrease of the displacement in the force-displacement-diagram when being subjected to a breaking strength test as described above (“Zwick Z 2.5” materials tester, constant speed), at least until the displacement d of upper jaw (1a) and lower jaw (1b) has been reduced by at least 0.1 mm, more preferably at least 0.2 mm, still more preferably at least 0.3 mm, yet more preferably at least 0.4 mm, even more preferably at least 0.5 mm, most preferably at least 0.6 mm, and in particular at least 0.7 mm, whereas the force measured at said displacement is within the range of from 5.0 N to 250 N, more preferably from 7.5 N to 225 N, still more preferably from 10 N to 200 N, yet more preferably from 15 N to 175 N, even more preferably from 20 N to 150 N, most preferably from 25 N to 125 N, and in particular from 30 N to 100 N.

In yet another embodiment, the particle(s) contained in the dosage form according to the invention have a deformability such that they are deformed without being fractured when subjected to a constant force of e.g. 50 N, 100 N, 200 N, 300 N, 400 N, 500 N or 600 N in a breaking strength test as described above (“Zwick Z 2.5” materials tester, constant force), until the displacement d of upper jaw (1a) and lower jaw (1b) is reduced so that no further deformation takes place at said constant force, whereas at this equilibrated state the displacement d of upper jaw (1a) and lower jaw (1b) is at most 90% of the original displacement d₀ (i.e. d≦0.9·d₀), preferably at most 80% of the original displacement d₀ (i.e. d≦0.8·d₀), more preferably at most 70% of the original displacement d₀ (i.e. d≦0.7·d₀), still more preferably at most 60% of the original displacement d₀ (i.e. d≦0.6·d₀), yet more preferably at most 50% of the original displacement d₀ (i.e. d≦0.5·d₀), even more preferably at most 40% of the original displacement d₀ (i.e. d≦0.4·d₀), most preferably at most 30% of the original displacement d₀ (i.e. d≦0.3·d₀), and in particular at most 20% of the original displacement d₀ (i.e. d≦0.2·d₀), or at most 15% of the original displacement d₀ (i.e. d≦0.15·d₀), at most 10% of the original displacement d₀ (i.e. d≦0.1·d₀), or at most 5% of the original displacement d₀ (i.e. d≦0.05·d₀).

Preferably, the particle(s) contained in the dosage form according to the invention have a deformability such that they are deformed without being fractured when subjected to a constant force of e.g. 50 N, 100 N, 200 N, 300 N, 400 N, 500 N or 600 N in a breaking strength test as described above (“Zwick Z 2.5” materials tester, constant force), until the displacement d of upper jaw (1a) and lower jaw (1b) is reduced so that no further deformation takes place at said constant force, whereas at this equilibrated state the displacement d of upper jaw (1a) and lower jaw (1b) is at most 0.80 mm or at most 0.75 mm, preferably at most 0.70 mm or at most 0.65 mm, more preferably at most 0.60 mm or at most 0.55 mm, still more preferably at most 0.50 mm or at most 0.45 mm, yet more preferably at most 0.40 mm or at most 0.35 mm, even more preferably at most 0.30 mm or at most 0.25 mm, most preferably at most 0.20 mm or at most 0.15 mm and in particular at most 0.10 or at most 0.05 mm.

In another embodiment, the particle(s) contained in the dosage form according to the invention have a deformability such that they are deformed without being fractured when subjected to a constant force of e.g. 50 N, 100 N, 200 N, 300 N, 400 N, 500 N or 600 N in a breaking strength test as described above (“Zwick Z 2.5” materials tester, constant force), until the displacement d of upper jaw (1a) and lower jaw (1b) is reduced so that no further deformation takes place at said constant force, whereas at this equilibrated state the displacement d of upper jaw (1a) and lower jaw (1b) is at least 5% of the original displacement d₀ (i.e. d≧0.05·d₀), preferably at least 10% of the original displacement d₀ (i.e. d≧0.1·d₀), more preferably at least 15% of the original displacement d₀ (i.e. d≧0.15·d₀), still more preferably at least 20% of the original displacement d₀ (i.e. d≧0.2·d₀), yet more preferably at least 30% of the original displacement d₀ (i.e. d≧0.3·d₀), even more preferably at least 40% of the original displacement d₀ (i.e. d≧0.4·d₀), most preferably at least 50% of the original displacement d₀ (i.e. d≧0.5·d₀), and in particular at least 60% of the original displacement d₀ (i.e. d≧0.6·d₀), or at least 70% of the original displacement d₀ (i.e. d≧0.7·d₀), at least 80% of the original displacement d₀ (i.e. d≧0.8·d₀), or at least 90% of the original displacement d₀ (i.e. d≧0.9·d₀).

Preferably, the particle(s) contained in the dosage form according to the invention have a deformability such that they are deformed without being fractured when subjected to a constant force of e.g. 50 N, 100 N, 200 N, 300 N, 400 N, 500 N or 600 N in a breaking strength test as described above (“Zwick Z 2.5” materials tester, constant force), until the displacement d of upper jaw (1a) and lower jaw (1b) is reduced so that no further deformation takes place at said constant force, whereas at this equilibrated state the displacement d of upper jaw (1a) and lower jaw (1b) is at least 0.05 mm or at least 0.10 mm, preferably at least 0.15 mm or at least 0.20 mm, more preferably at least 0.25 mm or at least 0.30 mm, still more preferably at least 0.35 mm or at least 0.40 mm, yet more preferably at least 0.45 mm or at least 0.50 mm, even more preferably at least 0.55 mm or at least 0.60 mm, most preferably at least 0.65 mm or at least 0.70 mm and in particular at least 0.75 or at least 0.80 mm.

The dosage form according to the invention preferably contains no antagonists for the pharmacologically active ingredient a, preferably no antagonists against psychotropic substances, in particular no antagonists against opioids. Antagonists suitable for a given pharmacologically active ingredient a are known to the person skilled in the art and may be present as such or in the form of corresponding derivatives, in particular esters or ethers, or in each case in the form of corresponding physiologically acceptable compounds, in particular in the form of the salts or solvates thereof. The dosage form according to the invention preferably contains no antagonists selected from among the group comprising naloxone, naltrexone, nalmefene, nalide, nalmexone, nalorphine or naluphine, in each case optionally in the form of a corresponding physiologically acceptable compound, in particular in the form of a base, a salt or solvate; and no neuroleptics, for example a compound selected from among the group comprising haloperidol, promethacine, fluphenazine, perphenazine, levomepromazine, thioridazine, perazine, chlorpromazine, chlorprothixine, zuclopenthixol, flupentixol, prothipendyl, zotepine, benperidol, pipamperone, melperone and bromperidol.

Further, the dosage form according to the invention preferably also contains no bitter substance. Bitter substances and the quantities effective for use may be found in US-2003/0064099 A1, the corresponding disclosure of which should be deemed to be the disclosure of the present application and is hereby introduced as a reference. Examples of bitter substances are aromatic oils, such as peppermint oil, eucalyptus oil, bitter almond oil, menthol, fruit aroma substances, aroma substances from lemons, oranges, limes, grapefruit or mixtures thereof, and/or denatonium benzoate.

The dosage form according to the invention accordingly preferably contains neither antagonists for the pharmacologically active ingredient a nor bitter substances.

In particularly preferred embodiments, the dosage form according to the invention comprises a multitude of particles A which
amount to a total number within the range of from 20 to 600; and/or
are made from substantially the same mixture of ingredients; and/or
have substantially of the same size, shape, weight and composition; and/or
have cylindrical shape; and/or
have substantially the same breaking strength;
have a breaking strength of at least 300 N; and/or
have an average individual weight within the range of from 0.1 mg to 5 mg; and/or
have a total weight within the range of from 10 mg to 500 mg; and/or
amount to a total content within the range of from 10 wt.-% to 80 wt.-%, based on the total weight of the dosage form; and/or
are tamper-resistant as such so that they also provide tamper-resistance after they have been separated from the remaining constituents of the dosage form; and/or
contain the total amount of the pharmacologically active ingredient a that is contained in the dosage form; and/or
have substantially the same content of pharmacologically active ingredient a; and/or
show substantially the same in vitro release profile; and/or
after 30 min under in vitro conditions have released at least 80 wt.-% of the pharmacologically active ingredient a that was originally contained in the dosage form; and/or
are thermoformed by hot-melt extrusion.

The dosage form according to the invention comprises at least a portion of the pharmacologically active ingredient b outside the particle(s) A in form of a powder.

In a preferred embodiment, the total amount of the pharmacologically active ingredient b that is contained in the dosage form according to the invention is contained outside the particle(s) A in form of a powder.

In another preferred embodiment, a portion b_{P} of the total amount of the pharmacologically active ingredient b that is contained in the dosage form according to the invention is contained outside the particle(s) A in form of a powder, whereas the remainder of the pharmacologically active ingredient b is contained elsewhere in the dosage form according to the invention.

When a portion of the pharmacologically active ingredient b is present in the one or more particle(s) A, said portion is referred to as “portion b_{A}”. Said portion b_{A} is neither contained in particle(s) B, nor is it contained in a coating of particle(s) A, nor is it present in form of a powder, nor is it present in form of granules.

When a portion of the pharmacologically active ingredient b is present outside the particle(s) A in one or more particle(s) B, said portion is referred to as “portion b_{B}”. Said portion b_{B} is neither contained in particle(s) A, nor is it contained in a coating of particle(s) A, nor is it present in form of a powder, nor is it present in form of granules.

When a portion of the pharmacologically active ingredient b is present outside the particle(s) A in a coating of particle(s) A, said portion is referred to as “portion b_{C}”. Said portion b_{C} is neither contained in particle(s) A, nor is it contained in particle(s) A, nor is it present in form of a powder, nor is it present in form of granules.

When a portion of the pharmacologically active ingredient b is present outside the particle(s) A in form of a granules, said portion is referred to as “portion b_{G}”. Said portion b_{G} is neither contained in particle(s) A, nor is it contained in a coating of particle(s) A, nor is it contained in particle(s) B, nor is it present in form of a powder.

When a portion of the pharmacologically active ingredient b is present outside the particle(s) A in form of a powder, said portion is referred to as “portion b_{P}”. Said portion b_{P} is neither contained in particle(s) A, nor is it contained in a coating of particle(s) A, nor is it contained in particle(s) B, nor is it present in form of granules.

Preferably, when the total amount of the pharmacologically active ingredient b is divided into portions that are present at different locations of the dosage form, the total amount of the pharmacologically active ingredient b is preferably divided in not more than three portions, more preferably not more than two portions.

Thus, when the total amount of the pharmacologically active ingredient b is divided into two portions, portion b_{P} is present in form of a powder outside of particle(s) A, whereas preferably the entire remainder amount of the pharmacologically active ingredient b, which is not present in form of a powder outside of particle(s) A, is present either as portion b_{A} in the particle(s) A, or as portion b_{B} in particle(s) B, or as portion b_{C} in a coating of particle(s) A, or as portion b_{G} outside particle(s) A in form of granules.

Preferably, the relative weight ratio of portion b_{P} to portion b_{A}, or the relative weight ratio of portion b_{P} to portion b_{B}, or the relative weight ratio of portion b_{P} to portion b_{C}, or the relative weight ratio of portion b_{P} to portion b_{G}, is within the range of from 100:1 to 1:100, more preferably 50:1 to 1:50, still more preferably 10:1 to 1:10, yet more preferably 5:1 to 1:5.

In a preferred embodiment, the weight of portion b_{P} is greater than the weight of portion b_{A}, or the weight of portion b_{P} is greater than the weight of portion b_{B}, or the weight of portion b_{P} is greater than the weight of portion b_{C}, or the weight of portion b_{P} is greater than the weight of portion b_{G}.

In another preferred embodiment, the weight of portion b_{A} is greater than the weight of portion b_{P}, or the weight of portion b_{B} is greater than the weight of portion b_{P}, or the weight of portion b_{C} is greater than the weight of portion b_{P}, or the weight of portion b_{G} is greater than the weight of portion b_{P}.

Particularly preferred distributions of the pharmacologically active ingredient a and the pharmacologically active ingredient b in the dosage form are summarized as embodiments X¹ to X²⁵ here the table below:

| | pharma- | pharma- | pharma- | pharma- | pharma- | |
|---|---|---|---|---|---|---|
| | cologically | cologically | cologically | cologically | cologically | pharma- |
| | active | active | active | active | active | cologically |
| | ingredient a | ingredient b | ingredient b | ingredient b | ingredient b | active ingredient |
| | in particle(s) | in particle(s) | in particle(s) | in coating of | in form of | b in form of |
| | A | A | B | particle(s) A | a powder | granules |
| X¹ | a_{Σ} | b_{Σ} | — | — | — | — |
| X² | a_{Σ} | — | b_{Σ} | — | — | — |
| X³ | a_{Σ} | — | — | b_{Σ} | — | — |
| X⁴ | a_{Σ} | — | — | — | b_{Σ} | — |
| X⁵ | a_{Σ} | — | — | — | — | b_{Σ} |
| X⁶ | a_{Σ} | b_{A} | b_{B} | — | — | — |
| X⁷ | a_{Σ} | b_{A} | — | b_{C} | — | — |
| X⁸ | a_{Σ} | b_{A} | — | — | b_{P} | — |
| X⁹ | a_{Σ} | b_{A} | — | — | — | b_{G} |
| X¹⁰ | a_{Σ} | — | b_{B} | b_{C} | — | — |
| X¹² | a_{Σ} | — | b_{B} | — | b_{P} | — |
| X¹³ | a_{Σ} | — | b_{B} | — | — | b_{G} |
| X¹⁴ | a_{Σ} | — | — | b_{C} | b_{P} | — |
| X¹⁵ | a_{Σ} | — | — | b_{C} | — | b_{G} |
| X¹⁶ | a_{Σ} | b_{A} | b_{B} | b_{C} | — | — |
| X¹⁷ | a_{Σ} | b_{A} | b_{B} | — | b_{P} | — |
| X¹⁸ | a_{Σ} | b_{A} | b_{B} | — | — | b_{G} |
| X¹⁹ | a_{Σ} | b_{A} | — | b_{C} | b_{P} | — |
| X²⁰ | a_{Σ} | b_{A} | — | b_{C} | — | b_{G} |
| X²¹ | a_{Σ} | b_{A} | — | — | b_{P} | b_{G} |
| X²² | a_{Σ} | — | b_{B} | b_{C} | b_{P} | — |
| X²³ | a_{Σ} | — | b_{B} | b_{C} | — | b_{G} |
| X²⁴ | a_{Σ} | — | b_{B} | — | b_{P} | b_{G} |
| X²⁵ | a_{Σ} | — | — | b_{C} | b_{P} | b_{G} |

a_{Σ} refers to the total amount of the pharmacologically active ingredient a
b_{Σ} refers to the total amount of the pharmacologically active ingredient b
b_{A} refers to a portion of the pharmacologically active ingredient b that is contained in particle(s) A
b_{B} refers to a portion of the pharmacologically active ingredient b that is contained in particle(s) B
b_{C} refers to a portion of the pharmacologically active ingredient b that is contained in a coating of particle(s) A
b_{G} refers to a portion of the pharmacologically active ingredient b that is contained in granules
b_{P} refers to a portion of the pharmacologically active ingredient b that is contained in form of a powder

According to the present invention, at least a portion of the pharmacologically active ingredient b (namely portion b_{P}) is contained outside particle(s) A in form of a powder.

In a preferred embodiment of the dosage form according to the invention, the portion b_{P} consists of the pharmacologically active ingredient b.

For the purpose of the specification, “powder” refers to any dry, bulk solid composed of a large number of very fine particles that may but do not need to flow freely when shaken or tilted.

In a preferred embodiment, the content of portion b_{P} relative to the total content of the pharmacologically active ingredient b that is contained in the dosage form according to the invention is at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-%, or about 100 wt.-%.

In another preferred embodiment, the content of portion b_{P} relative to the total content of the pharmacologically active ingredient b that is contained in the dosage form according to the invention is not more than 90 wt.-%, or not more than 80 wt.-%, or not more than 70 wt.-%, or not more than 60 wt.-%, or not more than 50 wt.-%, or not more than 40 wt.-%, or not more than 30 wt.-%, or not more than 20 wt.-%, or not more than 10 wt.-%.

It is preferred that the dosage form is a capsule where the powder of the pharmacologically active ingredient b is loosely contained in the capsule together with particle(s) A and optionally further ingredients.

The powder provides fast release, preferably immediate release of the pharmacologically active ingredient b. Preferably, after 30 min under in vitro conditions, the dosage form has released at least 80 wt.-% of the pharmacologically active ingredient b that was originally contained in the dosage form in form of a powder (portion b_{P}). Compared to optionally present portions b_{A}, b_{B} and b_{C}, it has been found that the powder provides comparatively fast release. In preferred embodiments, under in vitro conditions at least 80 wt.-% of the pharmacologically active ingredient b that was originally contained in the dosage form in form of a powder have been released after 28 min, or after 26 min, or after 24 min, or after 22 min, or after 20 min, or after 18 min, or after 16 min, or after 14 min, or after 12 min, or after 10 min.

In a preferred embodiment of the dosage form according to the invention, a portion b_{P} of the pharmacologically active ingredient b is contained outside the particle(s) A in form of a powder and wherein a portion b_{A} of the pharmacologically active ingredient b, preferably the remainder, is contained in particle(s) A.

In a preferred embodiment, the content of portion b_{A} relative to the total content of the pharmacologically active ingredient b that is contained in the dosage form according to the invention is at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-%, or about 100 wt.-%.

In another preferred embodiment, the content of portion b_{A} relative to the total content of the pharmacologically active ingredient b that is contained in the dosage form according to the invention is not more than 90 wt.-%, or not more than 80 wt.-%, or not more than 70 wt.-%, or not more than 60 wt.-%, or not more than 50 wt.-%, or not more than 40 wt.-%, or not more than 30 wt.-%, or not more than 20 wt.-%, or not more than 10 wt.-%.

In a preferred embodiment, the content of pharmacologically active ingredient b in the particle(s) A is within the range of from 2.0±1.9 wt.-%, or 2.5±2.4 wt.-%, or 3.0±2.9 wt.-%, or 3.5±3.4 wt.-%, or 4.0±3.9 wt.-%, or 4.5±4.4 wt.-%, or 5.0±4.9 wt.-%, or 5.5±5.4 wt.-%, or 6.0±5.9 wt.-%; more preferably 2.0±1.7 wt.-%, or 2.5±2.2 wt.-%, or 3.0±2.6 wt.-%, or 3.5±3.1 wt.-%, or 4.0±3.5 wt.-%, or 4.5±4.0 wt.-%, or 5.0±4.4 wt.-%, or 5.5±4.9 wt.-%, or 6.0±5.3 wt.-%, or 6.5±5.8 wt.-%, or 7.0±6.3 wt.-%, or 7.5±6.9 wt.-%, or 8.0±7.4 wt.-%, or 8.5±8.0 wt.-%, or 9.0±8.5 wt.-%, or 9.5±9.0 wt.-%, or 10±9.5 wt.-%, or 11±10 wt.-%, or 12±11 wt.-%, or 13±12 wt.-%, or 14±13 wt.-%, or 15±14 wt.-%; still more preferably 2.0±1.5 wt.-%, or 2.5±2.0 wt.-%, or 3.0±2.3 wt.-%, or 3.5±2.8 wt.-%, or 4.0±3.1 wt.-%, or 4.5±3.6 wt.-%, or 5.0±3.9 wt.-%, or 5.5±4.4 wt.-%, or 6.0±4.7 wt.-%, or 6.5±5.2 wt.-%, or 7.0±5.8 wt.-%, or 7.5±6.2 wt.-%, or 8.0±6.8 wt.-%, or 8.5±7.0 wt.-%, or 9.0±7.5 wt.-%, or 9.5±8.0 wt.-%, or 10±9.0 wt.-%, or 11±9.5 wt.-%, or 12±10 wt.-%, or 13±11 wt.-%, or 14±12 wt.-%, or 15±13 wt.-%; yet more preferably 2.0±1.3 wt.-%, or 2.5±1.8 wt.-%, or 3.0±2.0 wt.-%, or 3.5±2.5 wt.-%, or 4.0±2.7 wt.-%, or 4.5±3.2 wt.-%, or 5.0±3.4 wt.-%, or 5.5±3.9 wt.-%, or 6.0±4.1 wt.-%, or 6.5±4.7 wt.-%, or 7.0±5.2 wt.-%, or 7.5±5.7 wt.-%, or 8.0±6.2 wt.-%, or 8.5±6.0 wt.-%, or 9.0±6.5 wt.-%, or 9.5±7.0 wt.-%, or 10±8.5 wt.-%, or 11±9 wt.-%, or 12±10 wt.-%, or 13±11 wt.-%, or 14±12 wt.-%, or 15±13 wt.-%; even more preferably 2.0±1.1 wt.-%, or 2.5±1.6 wt.-%, or 3.0±1.7 wt.-%, or 3.5±2.2 wt.-%, or 4.0±2.4 wt.-%, or 4.5±2.8 wt.-%, or 5.0±2.9 wt.-%, or 5.5±3.4 wt.-%, or 6.0±3.5 wt.-%, or 6.5±4.2 wt.-%, or 7.0±4.7 wt.-%, or 7.5±5.2 wt.-%, or 8.0±5.7 wt.-%, or 8.5±5.0 wt.-%, or 9.0±5.5 wt.-%, or 9.5±6.0 wt.-%, or 10±6.5 wt.-%, or 11±8 wt.-%, or 12±9 wt.-%, or 13±10 wt.-%, or 14±11 wt.-%, or 15±12 wt.-%; most preferably 2.0±0.9 wt.-%, or 2.5±1.4 wt.-%, or 3.0±1.4 wt.-%, or 3.5±1.9 wt.-%, or 4.0±2.1 wt.-%, or 4.5±2.4 wt.-%, or 5.0±2.4 wt.-%, or 5.5±2.9 wt.-%, or 6.0±2.9 wt.-%, or 6.5±3.2 wt.-%, or 7.0±3.7 wt.-%, or 7.5±4.2 wt.-%, or 8.0±4.7 wt.-%, or 8.5±4.0 wt.-%, or 9.0±4.5 wt.-%, or 9.5±5.0 wt.-%, or 10±5.5 wt.-%, or 11±7 wt.-%, or 12±8 wt.-%, or 13±9 wt.-%, or 14±10 wt.-%, or 15±11 wt.-%; and in particular 2.0±0.7 wt.-%, or 2.5±1.2 wt.-%, or 3.0±1.1 wt.-%, or 3.5±1.6 wt.-%, or 4.0±1.8 wt.-%, or 4.5±2.0 wt.-%, or 5.0±1.9 wt.-%, or 5.5±2.4 wt.-%, or 6.0±2.3 wt.-%, or 6.5±2.7 wt.-%, or 7.0±3.2 wt.-%, or 7.5±3.7 wt.-%, or 8.0±4.2 wt.-%, or 8.5±2.0 wt.-%, or 9.0±2.5 wt.-%, or 9.5±3.0 wt.-%, or 10±3.5 wt.-%, or 11±4.0 wt.-%, or 12±5.0 wt.-%, or 13±6.0 wt.-%, or 14±7.0 wt.-%, or 15±8.0 wt.-%; in each case based on the total weight of the particle(s) A.

The particle(s) A provide fast release, preferably immediate release of the pharmacologically active ingredient b. Preferably, after 30 min under in vitro conditions, the particle(s) A have released at least 80 wt.-% of the pharmacologically active ingredient b that was originally contained in particle(s) A (portion b_{A}).

In a preferred embodiment of the dosage form according to the invention, a portion b_{P} of the pharmacologically active ingredient b is contained outside the particle(s) A in form of a powder and wherein a portion b_{C} of the pharmacologically active ingredient b, preferably the remainder, is contained in a coating of particle(s) A.

The particle(s) A according to the invention are preferably film coated with conventional film coating compositions. Such film coating compositions are then preferably mixed with portion b_{C} of the pharmacologically active ingredient b and applied to the outer surface of particle(s) A.

In a preferred embodiment, the content of portion b_{C} relative to the total content of the pharmacologically active ingredient b that is contained in the dosage form according to the invention is at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-%, or about 100 wt.-%.

In another preferred embodiment, the content of portion b_{C} relative to the total content of the pharmacologically active ingredient b that is contained in the dosage form according to the invention is not more than 90 wt.-%, or not more than 80 wt.-%, or not more than 70 wt.-%, or not more than 60 wt.-%, or not more than 50 wt.-%, or not more than 40 wt.-%, or not more than 30 wt.-%, or not more than 20 wt.-%, or not more than 10 wt.-%.

In a preferred embodiment, the content of pharmacologically active ingredient b in the coating of the particle(s) A is within the range of from 2.0±1.9 wt.-%, or 2.5±2.4 wt.-%, or 3.0±2.9 wt.-%, or 3.5±3.4 wt.-%, or 4.0±3.9 wt.-%, or 4.5±4.4 wt.-%, or 5.0±4.9 wt.-%, or 5.5±5.4 wt.-%, or 6.0±5.9 wt.-%; more preferably 2.0±1.7 wt.-%, or 2.5±2.2 wt.-%, or 3.0±2.6 wt.-%, or 3.5±3.1 wt.-%, or 4.0±3.5 wt.-%, or 4.5±4.0 wt.-%, or 5.0±4.4 wt.-%, or 5.5±4.9 wt.-%, or 6.0±5.3 wt.-%, or 6.5±5.8 wt.-%, or 7.0±6.3 wt.-%, or 7.5±6.9 wt.-%, or 8.0±7.4 wt.-%, or 8.5±8.0 wt.-%, or 9.0±8.5 wt.-%, or 9.5±9.0 wt.-%, or 10±9.5 wt.-%, or 11±10 wt.-%, or 12±11 wt.-%, or 13±12 wt.-%, or 14±13 wt.-%, or 15±14 wt.-%; still more preferably 2.0±1.5 wt.-%, or 2.5±2.0 wt.-%, or 3.0±2.3 wt.-%, or 3.5±2.8 wt.-%, or 4.0±3.1 wt.-%, or 4.5±3.6 wt.-%, or 5.0±3.9 wt.-%, or 5.5±4.4 wt.-%, or 6.0±4.7 wt.-%, or 6.5±5.2 wt.-%, or 7.0±5.8 wt.-%, or 7.5±6.2 wt.-%, or 8.0±6.8 wt.-%, or 8.5±7.0 wt.-%, or 9.0±7.5 wt.-%, or 9.5±8.0 wt.-%, or 10±9.0 wt.-%, or 11±9.5 wt.-%, or 12±10 wt.-%, or 13±11 wt.-%, or 14±12 wt.-%, or 15±13 wt.-%; yet more preferably 2.0±1.3 wt.-%, or 2.5±1.8 wt.-%, or 3.0±2.0 wt.-%, or 3.5±2.5 wt.-%, or 4.0±2.7 wt.-%, or 4.5±3.2 wt.-%, or 5.0±3.4 wt.-%, or 5.5±3.9 wt.-%, or 6.0±4.1 wt.-%, or 6.5±4.7 wt.-%, or 7.0±5.2 wt.-%, or 7.5±5.7 wt.-%, or 8.0±6.2 wt.-%, or 8.5±6.0 wt.-%, or 9.0±6.5 wt.-%, or 9.5±7.0 wt.-%, or 10±8.5 wt.-%, or 11±9 wt.-%, or 12±10 wt.-%, or 13±11 wt.-%, or 14±12 wt.-%, or 15±13 wt.-%; even more preferably 2.0±1.1 wt.-%, or 2.5±1.6 wt.-%, or 3.0±1.7 wt.-%, or 3.5±2.2 wt.-%, or 4.0±2.4 wt.-%, or 4.5±2.8 wt.-%, or 5.0±2.9 wt.-%, or 5.5±3.4 wt.-%, or 6.0±3.5 wt.-%, or 6.5±4.2 wt.-%, or 7.0±4.7 wt.-%, or 7.5±5.2 wt.-%, or 8.0±5.7 wt.-%, or 8.5±5.0 wt.-%, or 9.0±5.5 wt.-%, or 9.5±6.0 wt.-%, or 10±6.5 wt.-%, or 11±8 wt.-%, or 12±9 wt.-%, or 13±10 wt.-%, or 14±11 wt.-%, or 15±12 wt.-%; most preferably 2.0±0.9 wt.-%, or 2.5±1.4 wt.-%, or 3.0±1.4 wt.-%, or 3.5±1.9 wt.-%, or 4.0±2.1 wt.-%, or 4.5±2.4 wt.-%, or 5.0±2.4 wt.-%, or 5.5±2.9 wt.-%, or 6.0±2.9 wt.-%, or 6.5±3.2 wt.-%, or 7.0±3.7 wt.-%, or 7.5±4.2 wt.-%, or 8.0±4.7 wt.-%, or 8.5±4.0 wt.-%, or 9.0±4.5 wt.-%, or 9.5±5.0 wt.-%, or 10±5.5 wt.-%, or 11±7 wt.-%, or 12±8 wt.-%, or 13±9 wt.-%, or 14±10 wt.-%, or 15±11 wt.-%; and in particular 2.0±0.7 wt.-%, or 2.5±1.2 wt.-%, or 3.0±1.1 wt.-%, or 3.5±1.6 wt.-%, or 4.0±1.8 wt.-%, or 4.5±2.0 wt.-%, or 5.0±1.9 wt.-%, or 5.5±2.4 wt.-%, or 6.0±2.3 wt.-%, or 6.5±2.7 wt.-%, or 7.0±3.2 wt.-%, or 7.5±3.7 wt.-%, or 8.0±4.2 wt.-%, or 8.5±2.0 wt.-%, or 9.0±2.5 wt.-%, or 9.5±3.0 wt.-%, or 10±3.5 wt.-%, or 11±4.0 wt.-%, or 12±5.0 wt.-%, or 13±6.0 wt.-%, or 14±7.0 wt.-%, or 15±8.0 wt.-%; in each case based on the total weight of the particle(s) A, or based on the total weight of the coating of the particle(s) A.

The coating of particle(s) A provides fast release, preferably immediate release of the pharmacologically active ingredient b. Preferably, after 30 min under in vitro conditions, the coating of particle(s) A has released at least 80 wt.-% of the pharmacologically active ingredient b that was originally contained in the coating of particle(s) A (portion b_{C}).

In a preferred embodiment of the dosage form according to the invention, a portion b_{P} of the pharmacologically active ingredient b is contained outside the particle(s) A in form of a powder and wherein a portion b_{B} of the pharmacologically active ingredient b, preferably the remainder, is contained in one or more particle(s) B differing from particle(s) A.

Particle(s) B of the dosage form differ from particle(s) A of the dosage form. In a preferred embodiment, however, particle(s) B are not visually distinguishable from particle(s) A so that a potential abuser is unable to manually separate particle(s) A from particle(s) B. According to this embodiment, particle(s) A and particle(s) B have substantially the same size, shape, color, weight, density, morphology, surface appearance, and the like. This embodiment is particularly advantageous when the pharmacologically active ingredient a is more prone to abuse than pharmacologically active ingredient b. Under these circumstances, all excipients contained in particle(s) B contribute to the overall tamper-resistance of the dosage form, e.g. with respect to resistance against solvent extraction. A potential abuse is unable to manually separate the tamper-resistant excipients that are contained in particle(s) B from the pharmacologically active ingredient a with potential for abuse that is contained in particle(s) B.

The particle(s) B provide fast release, preferably immediate release of the pharmacologically active ingredient b. Preferably, after 30 min under in vitro conditions, the particle(s) B have released at least 80 wt.-% of the pharmacologically active ingredient b that was originally contained in particle(s) B (portion b_{B}).

In a preferred embodiment, the dosage form according to the invention comprises a single particle B.

In another preferred embodiment, the dosage form according to the invention comprises a plurality of particles B. Preferably, the dosage form comprises at least 2, or at least 3, or at least 4, or at least 5 particles B. Preferably, the dosage form comprises not more than 10, or not more than 9, or not more than 8, or not more than 7 particles B.

Preferably, when the dosage form contains more than a single particle B, the individual particles B may be of the same or of different size, shape and/or composition. Preferably, all particles B are made from the same mixture of ingredients and are substantially of the same size and shape and composition.

All preferred embodiments that have been described above for particle(s) A of the dosage form according to the invention, especially with respect to number, size, shape, content in the dosage form, nature of constituents, quantity of constituents and functional properties (such as tamper resistance and release profile), analogously and independently also apply to particle(s) B of the dosage form according to the invention and thus are not repeated hereinafter. However, pharmacologically active ingredient a is to be replaced by pharmacologically active ingredient b.

Preferably, particles B
comprise a polymer matrix in which the portion b_{B} of the pharmacologically active ingredient b is embedded; and/or
have a breaking strength of at least 300 N.

In preferred embodiments, the content of the particle(s) B is at least 2.5 wt.-%, at least 5 wt.-%, at least 7.5 wt.-% or at least 10 wt.-%; at least 12.5 wt.-%, at least 15 wt.-%, at least 17.5 wt.-% or at least 20 wt.-%; at least 22.5 wt.-%, at least 25 wt.-%, at least 27.5 wt.-% or at least 30 wt.-%; at least 32.5 wt.-%, at least 35 wt.-%, at least 37.5 wt.-% or at least 40 wt.-%; more preferably at least 42.5 wt.-%, at least 45 wt.-%, at least 47.5 wt.-% or at least 50 wt.-%; still more preferably at least 52.5 wt.-%, at least 55 wt.-%, at least 57.5 wt.-% or at least 60 wt.-%; yet more preferably at least 62.5 wt.-%, at least 65 wt.-%, at least 67.5 wt.-% or at least 60 wt.-%; most preferably at least 72.5 wt.-%, at least 75 wt.-%, at least 77.5 wt.-% or at least 70 wt.-%; and in particular at least 82.5 wt.-%, at least 85 wt.-%, at least 87.5 wt.-% or at least 90 wt.-%; based on the total weight of the dosage form.

Preferably, the content of the particle(s) B is at most 90 wt.-%, at most 87.5 wt.-%, at most 85 wt.-%, or at most 82.5 wt.-%; more preferably at most 80 wt.-%, at most 77.5 wt.-%, at most 75 wt.-% or at most 72.5 wt.-%; still more preferably at most 70 wt.-%, at most 67.5 wt.-%, at most 65 wt.-% or at most 62.5 wt.-%; yet more preferably at most 60 wt.-%, at most 57.5 wt.-%, at most 55 wt.-% or at most 52.5 wt.-%; most preferably at most 50 wt.-%, at most 47.5 wt.-%, at most 45 wt.-% or at most 42.5 wt.-%; and in particular at most 40 wt.-%, at most 37.5 wt.-%, or at most 35 wt.-%; based on the total weight of the dosage form.

Preferably, the total content of the pharmacologically active ingredient b is within the range of from 0.01 to more than 99.99 wt.-%, more preferably 0.1 to 99.9 wt.-%, still more preferably 5 to 95 wt.-%, based on the total weight of the particle(s) B. In a preferred embodiment, the total content of the pharmacologically active ingredient b is within the range of from 20±6 wt.-%, 30±6 wt.-% or 40±6 wt.-%, more preferably 20±5 wt.-%, 30±5 wt.-% or 40±5 wt.-%, still more preferably 20±4 wt.-%, 30±4 wt.-% or 40±4 wt.-%, most preferably 20±3 wt.-%, 30±3 wt.-% or 40±3 wt.-% and in particular 20±2 wt.-%, 30±2 wt.-% or 40±2 wt.-%, based on the total weight of the particle(s) B. In another preferred embodiment, the total content of the pharmacologically active ingredient b is within the range of from 50±20 wt.-%, 60±20 wt.-%, 70±20 wt.-% or 80±20 wt.-%, more preferably 50±15 wt.-%, 60±15 wt.-%, 70±15 wt.-% or 80±15 wt.-%, still more preferably 50±12 wt.-%, 60±12 wt.-%, 70±12 wt.-% or 80±12 wt.-%, most preferably 50±10 wt.-%, 60±10 wt.-%, 70±10 wt.-% or 80±10 wt.-%, and in particular 50±5 wt.-%, 60±5 wt.-%, 70±5 wt.-% or 80±5 wt.-%, based on the total weight of the particle(s) B. In still another preferred embodiment, the total content of the pharmacologically active ingredient b is within the range of from 90±10 wt.-%, more preferably 90±8 wt.-%, still more preferably 90±6 wt.-%, most preferably 90±4 wt.-% and in particular 90±2 wt.-%, based on the total weight of the particle(s) B.

In particularly preferred embodiments, the dosage form according to the invention comprises a multitude of particle(s) B which
amount to a total number within the range of from 20 to 600; and/or
are made from substantially the same mixture of ingredients; and/or
have substantially of the same size, shape, weight and composition; and/or
have cylindrical shape; and/or
have substantially the same breaking strength;
have a breaking strength of at least 300 N; and/or
have an average individual weight within the range of from 0.1 mg to 5 mg; and/or
have a total weight within the range of from 10 mg to 500 mg; and/or
amount to a total content within the range of from 10 wt.-% to 80 wt.-%, based on the total weight of the dosage form; and/or
are tamper-resistant as such so that they also provide tamper-resistance after they have been separated from the remaining constituents of the dosage form; and/or
contain the total amount of the pharmacologically active ingredient b that is contained in the dosage form; and/or
have substantially the same content of pharmacologically active ingredient b; and/or
show substantially the same in vitro release profile; and/or
after 30 min under in vitro conditions have released at least 80 wt.-% of the pharmacologically active ingredient b that was originally contained in the dosage form; and/or
are thermoformed by hot-melt extrusion.

Preferably, the relative weight ratio of the particle(s) A to the particle(s) B in the dosage form is from 1:10 to 10:1, more preferably 1:8 to 8:1, still more preferably 1:7 to 6:1, even more preferably 1:6 to 5:1, yet more preferably 1:5 to 4:1, most preferably 1:4 to 3:1 and in particular 1:3 to 2:1 or 1:2 to 1:1, based on the total weight of the particle(s) A and on the total weight of the particle(s) B.

In a preferred embodiment of the dosage form according to the invention, a portion b_{P} of the pharmacologically active ingredient b is contained outside the particles A in form of a powder and wherein a portion b_{G} of the pharmacologically active ingredient b, preferably the remainder, is contained outside particles A in form of granules. The granules may be present in form of a heap of loose material, e.g. a capsule filling also comprising particle(s) A and optionally present particle(s) B, or as a compacted material that may form an outer matrix material of a tablet in which the particle(s) A and the optionally present particle(s) B are embedded.

In a preferred embodiment, the content of portion b_{G} relative to the total content of the pharmacologically active ingredient b that is contained in the dosage form according to the invention is at least 10 wt.-%, or at least 20 wt.-%, or at least 30 wt.-%, or at least 40 wt.-%, or at least 50 wt.-%, or at least 60 wt.-%, or at least 70 wt.-%, or at least 80 wt.-%, or at least 90 wt.-%, or about 100 wt.-%.

In another preferred embodiment, the content of portion b_{G} relative to the total content of the pharmacologically active ingredient b that is contained in the dosage form according to the invention is not more than 90 wt.-%, or not more than 80 wt.-%, or not more than 70 wt.-%, or not more than 60 wt.-%, or not more than 50 wt.-%, or not more than 40 wt.-%, or not more than 30 wt.-%, or not more than 20 wt.-%, or not more than 10 wt.-%.

The granules provide fast release, preferably immediate release of the pharmacologically active ingredient b. Preferably, after 30 min under in vitro conditions, the granules have released at least 80 wt.-% of the pharmacologically active ingredient b that was originally contained in the granules (portion b_{G}). Compared to optionally present portions b_{A}, b_{B} and b_{C}, it has been found that the granules provide comparatively fast release. In preferred embodiments, under in vitro conditions at least 80 wt.-% of the pharmacologically active ingredient b that was originally contained in the granules have been released after 28 min, or after 26 min, or after 24 min, or after 22 min, or after 20 min, or after 18 min, or after 16 min, or after 14 min, or after 12 min, or after 10 min.

In a preferred embodiment, the content of pharmacologically active ingredient b in the granules is within the range of from 40.00±35.00 wt.-%, more preferably 40.00±30.00 wt.-%, still more preferably 40.00±25.00 wt.-%, yet more preferably 40.00±20.00 wt.-%, even more preferably 40.00±15.00 wt.-%, most preferably 40.00±10.00 wt.-%, and in particular 40.00±5.00 wt.-%, based on the total weight of the granules.

In another preferred embodiment, the content of pharmacologically active ingredient b in the granules is within the range of from 50.00±35.00 wt.-%, more preferably 50.00±30.00 wt.-%, still more preferably 50.00±25.00 wt.-%, yet more preferably 50.00±20.00 wt.-%, even more preferably 50.00±15.00 wt.-%, most preferably 50.00±10.00 wt.-%, and in particular 50.00±5.00 wt.-%, based on the total weight of the granules.

In still another preferred embodiment, the content of pharmacologically active ingredient b in the granules is within the range of from 60.00±35.00 wt.-%, more preferably 60.00±30.00 wt.-%, still more preferably 60.00±25.00 wt.-%, yet more preferably 60.00±20.00 wt.-%, even more preferably 60.00±15.00 wt.-%, most preferably 60.00±10.00 wt.-%, and in particular 60.00±5.00 wt.-%, based on the total weight of the granules.

In yet another preferred embodiment, the content of pharmacologically active ingredient b in the granules is within the range of from 70.00±28.00 wt.-%, more preferably 70.00±24.00 wt.-%, still more preferably 70.00±20.00 wt.-%, yet more preferably 70.00±16.00 wt.-%, even more preferably 70.00±12.00 wt.-%, most preferably 70.00±8.00 wt.-%, and in particular 70.00±4.00 wt.-%, based on the total weight of the granules.

Preferably, the granules according to the invention comprise a filler or binder such as saccharides, e.g. lactose, sugar alcohols, e.g. mannitol, or cellulose and its derivatives, e.g. microcrystalline cellulose.

In a preferred embodiment, the content of filler/binder in the granules is within the range of from 20.00±18.00 wt.-%, more preferably 20.00±16.00 wt.-%, still more preferably 20.00±14.00 wt.-%, yet more preferably 20.00±12.00 wt.-%, even more preferably 20.00±10.00 wt.-%, most preferably 20.00±7.50 wt.-%, and in particular 20.00±5.00 wt.-%, based on the total weight of the granules.

In another preferred embodiment, the content of filler/binder in the granules is within the range of from 30.00±28.00 wt.-%, more preferably 30.00±24.00 wt.-%, still more preferably 30.00±20.00 wt.-%, yet more preferably 30.00±16.00 wt.-%, even more preferably 30.00±12.00 wt.-%, most preferably 30.00±8.00 wt.-%, and in particular 30.00±4.00 wt.-%, based on the total weight of the granules.

In still another preferred embodiment, the content of filler/binder in the granules is within the range of from 40.00±35.00 wt.-%, more preferably 40.00±30.00 wt.-%, still more preferably 40.00±25.00 wt.-%, yet more preferably 40.00±20.00 wt.-%, even more preferably 40.00±15.00 wt.-%, most preferably 40.00±10.00 wt.-%, and in particular 40.00±5.00 wt.-%, based on the total weight of the granules.

Preferably, the granules according to the invention comprise a disintegrant.

Suitable disintegrants are known to the skilled person and are preferably selected from the group consisting of polysaccharides, starches, starch derivatives, cellulose derivatives, polyvinylpyrrolidones, acrylates, gas releasing substances, and the mixtures of any of the foregoing.

Preferred starches include but are not limited to “standard starch” (e.g. native maize starch) and pregelatinized starch (e.g. starch 1500).

Preferred starch derivatives include but are not limited to sodium starch glycolate (carboxymethyl starch sodium, e.g. Vivastar®).

Preferred cellulose derivatives include but are not limited to croscarmellose sodium (=crosslinked sodium carboxymethylcellulose; e.g. Vivasol®), carmellose calcium (calcium carboxymethylcellulose), carmellose sodium (sodium carboxymethylcellulose), low substituted carmellose sodium (low substituted sodium carboxymethylcellulose; average degree of substitution (DS) 0.20 to 0.40, Mr 80,000 to 600,000 g/mol, CAS 9004-32-4, E 466), low substituted hydroxypropylcellulose (having a content of propyl groups within the range of from 5 to 16%; CAS 9004-64-2).

Preferred acrylates include but are not limited to carbopol.

Preferred polyvinylpyrrolidones include but are not limited to crospovidone (PVP Cl).

Preferred gas releasing substances include but are not limited to sodium bicarbonate.

Preferred disintegrants include but are not limited to crosslinked sodium carboxymethylcellulose (Na-CMC) (e.g. Crosscarmellose, Vivasol®, Ac-Di-Sol®); crosslinked casein (e.g. Esma-Spreng®); polysaccharide mixtures obtained from soybeans (e.g. Emcosoy®); maize starch or pretreated maize starch (e.g. Amijel®); alginic acid, sodium alginate, calcium alginate; polyvinylpyrrolidone (PVP) (e.g. Kollidone®, Polyplasdone®, Polydone®); crosslinked polyvinylpyrrolidone (PVP CI) (e.g. Polyplasdone® XL); starch and pretreated starch such as sodium carboxymethyl starch (=sodium starch glycolate, e.g. Explotab®, Prejel®, Primotab® ET, Starch® 1500, Ulmatryl®), and the mixtures thereof. Crosslinked polymers are particularly preferred disintegrants, especially crosslinked sodium carboxymethylcellulose (Na-CMC) or crosslinked polyvinylpyrrolidone (PVP CI).

Particularly preferred disintegrants are selected from the group consisting of
crosslinked sodium carboxymethylcellulose (Na-CMC) (e.g. Crosscarmellose, Vivasol®, Ac-Di-Sol®);
crosslinked casein (e.g. Esma-Spreng®);
alginic acid, sodium alginate, calcium alginate;
polysaccharide mixtures obtained from soybeans (e.g. Emcosoy®);
starch and pretreated starch such as sodium carboxymethyl starch (=sodium starch glycolate, e.g. Explotab®, Prejel®, Primotab® ET, Starch® 1500, Ulmatryl®);
maize starch or pretreated maize starch (e.g. Amijel®);
and mixtures of any of the foregoing.

Preferably, the content of the disintegrant is at least 6.0 wt.-%, at least 7.0 wt.-%, at least 8.0 wt.-%, at least 9.0 wt.-%, or at least 10 wt.-%, more preferably at least 12 wt.-%, still more preferably at least 14 wt.-%, yet more preferably at least 15 wt.-%, even more preferably at least 16 wt.-%, most preferably at least 18 wt.-%, and in particular at least 19 wt.-%, based on the total weight of the pharmaceutical dosage form and/or based on the total weight of the granules.

In a preferred embodiment, the content of disintegrant in the granules is within the range of from 4.00±3.50 wt.-%, more preferably 4.00±3.00 wt.-%, still more preferably 4.00±2.50 wt.-%, yet more preferably 4.00±2.00 wt.-%, even more preferably 4.00±1.50 wt.-%, most preferably 4.00±1.00 wt.-%, and in particular 4.00±3.00 wt.-%, based on the total weight of the granules.

In another preferred embodiment, the content of disintegrant in the granules is within the range of from 6.00±5.50 wt.-%, more preferably 6.00±5.00 wt.-%, still more preferably 6.00±4.50 wt.-%, yet more preferably 6.00±4.00 wt.-%, even more preferably 6.00±3.50 wt.-%, most preferably 6.00±2.50 wt.-%, and in particular 6.00±1.50 wt.-%, based on the total weight of the granules.

In still another preferred embodiment, the content of disintegrant in the granules is within the range of from 8.00±7.00 wt.-%, more preferably 8.00±6.00 wt.-%, still more preferably 8.00±5.00 wt.-%, yet more preferably 8.00±4.00 wt.-%, even more preferably 8.00±3.00 wt.-%, most preferably 8.00±2.00 wt.-%, and in particular 8.00±1.00 wt.-%, based on the total weight of the granules.

In yet another preferred embodiment, the content of disintegrant in the granules is within the range of from 10.00±9.00 wt.-%, more preferably 10.00±8.00 wt.-%, still more preferably 10.00±7.00 wt.-%, yet more preferably 10.00±6.00 wt.-%, even more preferably 10.00±5.00 wt.-%, most preferably 10.00±4.00 wt.-%, and in particular 10.00±3.00 wt.-%, based on the total weight of the granules.

Preferably, the granules according to the invention comprise a lubricant such as magnesium stearate or highly disperse silicium dioxide (e.g. Aerosil 200, Aerosil COK85).

In a preferred embodiment, the content of lubricant in the granules is within the range of from 2.00±1.80 wt.-%, more preferably 2.00±1.60 wt.-%, still more preferably 2.00±1.40 wt.-%, yet more preferably 2.00±1.20 wt.-%, even more preferably 2.00±1.00 wt.-%, most preferably 2.00±0.80 wt.-%, and in particular 2.00±0.60 wt.-%, based on the total weight of the granules.

In another preferred embodiment, the content of lubricant in the granules is within the range of from 4.00±3.50 wt.-%, more preferably 4.00±3.00 wt.-%, still more preferably 4.00±2.50 wt.-%, yet more preferably 4.00±2.00 wt.-%, even more preferably 4.00±1.50 wt.-%, most preferably 4.00±1.00 wt.-%, and in particular 4.00±3.00 wt.-%, based on the total weight of the granules.

In still another preferred embodiment, the content of lubricant in the granules is within the range of from 6.00±5.50 wt.-%, more preferably 6.00±5.00 wt.-%, still more preferably 6.00±4.50 wt.-%, yet more preferably 6.00±4.00 wt.-%, even more preferably 6.00±3.50 wt.-%, most preferably 6.00±2.50 wt.-%, and in particular 6.00±1.50 wt.-%, based on the total weight of the granules.

In yet another preferred embodiment, the content of lubricant in the granules is within the range of from 8.00±7.00 wt.-%, more preferably 8.00±6.00 wt.-%, still more preferably 8.00±5.00 wt.-%, yet more preferably 8.00±4.00 wt.-%, even more preferably 8.00±3.00 wt.-%, most preferably 8.00±2.00 wt.-%, and in particular 8.00±1.00 wt.-%, based on the total weight of the granules.

In a further preferred embodiment, the content of lubricant in the granules is within the range of from 10.00±9.00 wt.-%, more preferably 10.00±8.00 wt.-%, still more preferably 10.00±7.00 wt.-%, yet more preferably 10.00±6.00 wt.-%, even more preferably 10.00±5.00 wt.-%, most preferably 10.00±4.00 wt.-%, and in particular 10.00±3.00 wt.-%, based on the total weight of the granules.

Preferably, the granules according to the invention comprise a binder, such as a further polymer, preferably a cellulose ether such as hydroxypropylmethylcellulose. Preferred binders are selected from polysaccharides and their derivatives such as cellulose, cellulose derivatives, starches, starch derivatives and synthetic polymers such as polyvinylpyrrolidone (PVP). Preferred binders include but are not limited to
cellulose such as microcrystalline cellulose;
cellulose ethers, such as hydroxypropylcellulose (HPC), hydroxypropylmethylcellulose (HPMC, hypromellose);
starches, such as maize starch or pregelatinized starch; and
synthetic polymers such as polyvinylpyrrolidone.

In a preferred embodiment, the content of the binder, preferably the further polymer in the granules is within the range of from 1.50±1.40 wt.-%, more preferably 1.50±1.20 wt.-%, still more preferably 1.50±1.00 wt.-%, yet more preferably 1.50±0.80 wt.-%, even more preferably 1.50±0.60 wt.-%, most preferably 1.50±0.40 wt.-%, and in particular 1.50±0.20 wt.-%, based on the total weight of the granules.

In a preferred embodiment, the dosage form according to the invention is a tablet, wherein the particle(s) A are contained in a matrix of a matrix material. The “matrix material” is not to be confused with the “polymer matrix” of the particle(s) A and the optionally present particle(s) B. In the following, this preferred embodiment is referred to as the “preferred tablet according to the invention”.

When the preferred tablet according to the invention comprises particle(s) B, the following preferred embodiments described for particles(s) A may also analogously and independently apply to particle(s) B. Thus, in the following it is generally referred to “the particle(s)” when no specific distinction between particle(s) A and the optionally present particle(s) B is necessary, nevertheless implying the quality and quantity of particle(s) A and particle(s) B are still independent of one another.

The preferred tablet according to the invention comprises subunits having different morphology and properties, namely particle(s) and matrix material, wherein the particle(s) form a discontinuous phase within the matrix material. The particle(s) typically have mechanical properties that differ from the mechanical properties of the matrix material. Preferably, the particle(s) have a higher mechanical strength than the matrix material. The particle(s) within the preferred tablet according to the invention can be visualized by conventional means such as x-ray, solid state nuclear magnetic resonance spectroscopy, raster electron microscopy, terahertz spectroscopy and the like.

In the preferred tablet according to the invention, the particle(s) are incorporated in a matrix material. From a macroscopic perspective, the matrix material preferably forms a continuous phase in which the particle(s) are embedded as discontinuous phase.

Preferably, the matrix material is a homogenous coherent mass, preferably a homogeneous mixture of solid constituents, in which the particle(s) are embedded thereby spatially separating the particle(s) from one another. While it is possible that the surfaces of particle(s) are in contact or at least in very close proximity with one another, the plurality of particle(s) preferably cannot be regarded as a single continuous coherent mass within the preferred tablet according to the invention.

In other words, the preferred tablet according to the invention comprises the particle(s) as volume element(s) of a first type in which the pharmacologically active ingredient a and the polymer matrix, which preferably comprises polyalkylene oxide, are contained, preferably homogeneously, and the matrix material as volume element of a second type differing from the material that forms the particle(s), preferably containing neither pharmacologically active ingredient a/b nor polymer matrix, polyalkylene oxide, but optionally polyethylene glycol which differs from polyethylene oxide in its molecular weight.

When portion b_{P} of the pharmacologically active ingredient is present in form of a powder, said powder is a constituent of the matrix material of the preferred tablet according to the invention.

When portion b_{G} of the pharmacologically active ingredient is present in form of granules, said granules are a constituent of the matrix material of the preferred tablet according to the invention.

A purpose of the matrix material in the preferred tablet according to the invention is to ensure rapid disintegration and subsequent release of the pharmacologically active ingredients a and b from the disintegrated preferred tablet according to the invention, i.e. from the particle(s) A and optionally from particle(s) B, from the coating of particle(s) A, from the granules and from the powder, respectively. Thus, the matrix material preferably does not contain any excipient that might have a retardant effect on disintegration and drug release, respectively. Thus, the matrix material preferably does not contain any polymer that is typically employed as matrix material in prolonged release formulations.

The preferred tablet according to the invention preferably comprises the matrix material in an amount of more than one third of the total weight of the preferred tablet according to the invention. Thus, the polymer matrix which preferably comprises polyalkylene oxide and which is contained in the particle(s) A of the preferred tablet according to the invention is preferably not also contained in the matrix material.

Preferably, the pharmacologically active ingredient a which is contained in the particle(s) A of the preferred tablet according to the invention is preferably not also contained in the matrix material. Thus, in a preferred embodiment, the total amount of pharmacologically active ingredient a contained in the preferred tablet according to the invention is present in the particle(s) A which form a discontinuous phase within the matrix material; and the matrix material forming a continuous phase does not contain any pharmacologically active ingredient a.

Preferably, the pharmacologically active ingredient b, at least a portion of which is preferably present as a powder and/or in form of granules, is contained in the matrix material, whereas compaction of the preferred tablet according to the invention has typically caused compaction of said powder and/or granules, typically in admixture with the other constituents of the matrix material.

Preferably, the content of the matrix material is at least 35 wt.-%, at least 37.5 wt.-% or at least 40 wt.-%; more preferably at least 42.5 wt.-%, at least 45 wt.-%, at least 47.5 wt.-% or at least 50 wt.-%; still more preferably at least 52.5 wt.-%, at least 55 wt.-%, at least 57.5 wt.-% or at least 60 wt.-%; yet more preferably at least 62.5 wt.-%, at least 65 wt.-%, at least 67.5 wt.-% or at least 60 wt.-%; most preferably at least 72.5 wt.-%, at least 75 wt.-%, at least 77.5 wt.-% or at least 70 wt.-%; and in particular at least 82.5 wt.-%, at least 85 wt.-%, at least 87.5 wt.-% or at least 90 wt.-%; based on the total weight of the preferred tablet according to the invention.

Preferably, the content of the matrix material is at most 90 wt.-%, at most 87.5 wt.-%, at most 85 wt.-%, or at most 82.5 wt.-%; more preferably at most 80 wt.-%, at most 77.5 wt.-%, at most 75 wt.-% or at most 72.5 wt.-%; still more preferably at most 70 wt.-%, at most 67.5 wt.-%, at most 65 wt.-% or at most 62.5 wt.-%; yet more preferably at most 60 wt.-%, at most 57.5 wt.-%, at most 55 wt.-% or at most 52.5 wt.-%; most preferably at most 50 wt.-%, at most 47.5 wt.-%, at most 45 wt.-% or at most 42.5 wt.-%; and in particular at most 40 wt.-%, at most 37.5 wt.-%, or at most 35 wt.-%; based on the total weight of the preferred tablet according to the invention.

In a preferred embodiment, the content of the matrix material is within the range of 40±5 wt.-%, more preferably 40±2.5 wt.-%, based on the total weight of the preferred tablet according to the invention. In another preferred embodiment, the content of the matrix material is within the range of 45±10 wt.-%, more preferably 45±7.5 wt.-%, still more preferably 45±5 wt.-%, and most preferably 45±2.5 wt.-%, based on the total weight of the preferred tablet according to the invention. In still another preferred embodiment, the content of the matrix material is within the range of 50±10 wt.-%, more preferably 50±7.5 wt.-%, still more preferably 50±5 wt.-%, and most preferably 50±2.5 wt.-%, based on the total weight of the preferred tablet according to the invention. In yet another preferred embodiment, the content of the matrix material is within the range of 55±10 wt.-%, more preferably 55±7.5 wt.-%, still more preferably 55±5 wt.-%, and most preferably 55±2.5 wt.-%, based on the total weight of the preferred tablet according to the invention.

Preferably, the matrix material is a mixture, preferably a homogeneous mixture of at least two different constituents, more preferably of at least three different constituents. In a preferred embodiment, all constituents of the matrix material are homogeneously distributed in the continuous phase that is formed by the matrix material.

According to a variant of the preferred tablet according to the invention, the particle(s) A may be incorporated in an outer matrix material formed by the optionally present particle(s) B and/or by the optionally present granules. From a macroscopic perspective, the outer matrix material formed by the particle(s) B preferably forms a continuous phase in which the particle(s) A are embedded.

For the purpose of definition, the “outer matrix material” preferably comprises or consists of the particle(s) B and/or the granules, and thus, preferably comprises the pharmacologically active ingredient b and optionally conventional pharmaceutical excipients which have already been described above.

Preferably, the outer matrix material is a homogenous powdery or coherent mass, preferably a homogeneous mixture of solid constituents, in which the particle(s) A are embedded. According to this embodiment, the particle(s) A are preferably spatially separated from one another. While it is possible that the surfaces of particle(s) A are in contact or at least in very close proximity with one another, the plurality of particle(s) A preferably cannot be regarded as a single continuous coherent mass within the dosage form.

In other words, when the particle(s) A are contained in an outer matrix material formed by the particle(s) B and/or the granules, the dosage form according to the invention preferably comprises the particle(s) A as volume elements of a first type and the outer matrix material formed by the particle(s) B and/or the granules as volume element of a second type differing from the material that forms the particle(s) A.

In a preferred embodiment, the particle(s) B exhibit a breaking strength that is lower than that of particle(s) A. Preferably, the particle(s) B exhibit a breaking strength within the range of from 0 N to at most 500 N. Preferably, the particle(s) B exhibit a breaking strength within the range of from 0 N to 450 N, more preferably 0 N to 400 N, still more preferably 0 N to 350 N, yet more preferably 0 N to 300 N, most preferably 0 N to 250 N and in particular 0 N to 200 N. In a preferred embodiment, the particle(s) B exhibits a breaking strength of at most 500 N, more preferably at most 300 N, still more preferably at most 250 N, yet more preferably at most 200 N, even more preferably at most 150 N, most preferably at most 100 N, and in particular at most 50 N.

Preferably, the breaking strength of the particle(s) A is relatively at least 50 N higher, more preferably at least 100 N higher, still more preferably at least 150 N higher, yet more preferably at least 200 N higher, even more preferably at least 250 N higher, most preferably at least 300 N higher, and in particular at least 350 N higher than the breaking strength of the particle(s) B.

The dosage form according to the invention may contain additional pharmaceutical excipients conventionally contained in dosage forms in conventional amounts, such as fillers, binders, dispersing agents, wetting agents, disintegrants, gelling agents, antioxidants, preservatives, lubricants, plasticizer, fillers, binders, and the like.

Said excipients may independently of one another be present in the particle(s) A, the matrix material of the preferred tablet according to the invention, the capsule filling, the optionally present particle(s) B, the optionally present coating of particle(s) A, and the optionally present granules, respectively.

The skilled person will readily be able to determine appropriate excipients as well as the quantities of each of these excipients. Specific examples of pharmaceutically acceptable carriers and excipients that may be used to formulate the dosage forms according to the invention are described in the Handbook of Pharmaceutical Excipients, American Pharmaceutical Association (1986).

Preferably, the particle(s), the coating, the outer matrix material, the capsule filling, and/or the granules independently comprise one or more fillers or binders. As many fillers can be regarded as binders and vice versa, for the purpose of the specification “filler/binder” refers to any excipient that is suitable as filler, binder or both. Thus, the particle(s), the coating, the outer matrix material, the capsule filling, and/or the granules independently preferably comprise a filler/binder.

Preferred fillers (=filler/binders) are selected from the group consisting of silicium dioxide (e.g. Aerosil®, microcrystalline cellulose (e.g. Avicel®, Elcema®, Emocel®, ExCel®, Vitacell®); cellulose ether (e.g. Natrosol®, Klucel®, Methocel®, Blanose®, Pharmacoat®, Viscontran®); mannitol; dextrines; dextrose; calciumhydrogen phosphate (e.g. Emcompress®); tricalcium phosphate, maltodextrine (e.g. Emdex®); lactose (e.g. Fast-Flow Lactose®; Ludipress®, Dosage Formtose®, Zeparox®); polyvinylpyrrolidone (PVP) (e.g. Kollidone®, Polyplasdone®, Polydone®); saccharose (e.g. Nu-Tab®, Sugar Tab®); magnesium salts (e.g. MgCO₃, MgO, MgSiO₃); starches and pretreated starches (e.g. Prejel®, Primotab® ET, Starch® 1500). Preferred binders are selected from the group consisting of alginates; chitosanes; and any of the fillers mentioned above (=fillers/binders).

Some fillers/binders may also serve other purposes. It is known, for example, that silicium dioxide exhibits excellent function as a glidant. Preferably, the particle(s), the coating, the outer matrix material, the capsule filling, and/or the granules independently comprise a glidant such as silicium dioxide.

In a preferred embodiment, the content of the filler/binder or mixture of fillers/binders in the particle(s), the coating, the outer matrix material, the capsule filling, and/or the granules independently is within the range of 50±25 wt.-%, more preferably 50±20 wt.-%, still more preferably 50±15 wt.-%, yet more preferably 50±10 wt.-%, most preferably 50±7.5 wt.-%, and in particular 50±5 wt.-%, based on the total weight of the particle(s), the coating, the outer matrix material, the capsule filling, and/or the granules, respectively. In another preferred embodiment, the content of the filler/binder or mixture of fillers/binders in the particle(s), the coating, the outer matrix material, the capsule filling, and/or the granules independently is within the range of 65±25 wt.-%, more preferably 65±20 wt.-%, still more preferably 65±15 wt.-%, yet more preferably 65±10 wt.-%, most preferably 65±7.5 wt.-%, and in particular 65±5 wt.-%, based on the total weight of the particle(s), the coating, the outer matrix material, the capsule filling, and/or the granules, respectively. In still another preferred embodiment, the content of the filler/binder or mixture of fillers/binders in the particle(s), the coating, the outer matrix material, the capsule filling, and/or the granules independently is within the range of 80±19 wt.-%, more preferably 80±17.5 wt.-%, still more preferably 80±15 wt.-%, yet more preferably 80±10 wt.-%, most preferably 80±7.5 wt.-%, and in particular 80±5 wt.-%, based on the total weight of the particle(s), the coating, the outer matrix material, the capsule filling, and/or the granules, respectively. In another preferred embodiment, the content of the filler/binder or mixture of fillers/binders in the particle(s), the coating, the outer matrix material, the capsule filling, and/or the granules independently is within the range of 90±9 wt.-%, more preferably 90±8 wt.-%, still more preferably 90±7 wt.-%, yet more preferably 90±6 wt.-%, most preferably 90±5 wt.-%, and in particular 90±4 wt.-%, based on the total weight of the particle(s), the coating, the outer matrix material, the capsule filling, and/or the granules, respectively.

In a preferred embodiment, the total content of the filler/binder or mixture of fillers/binders in the dosage form is within the range of 25±24 wt.-%, more preferably 25±20 wt.-%, still more preferably 25±16 wt.-%, yet more preferably 25±12 wt.-%, most preferably 25±8 wt.-%, and in particular 25±4 wt.-%, based on the total weight of dosage form. In another preferred embodiment, the total content of the filler/binder or mixture of fillers/binders in the dosage form is within the range of 30±29 wt.-%, more preferably 30±25 wt.-%, still more preferably 30±20 wt.-%, yet more preferably 30±15 wt.-%, most preferably 30±10 wt.-%, and in particular 30±5 wt.-%, based on the total weight of dosage form. In still another preferred embodiment, the total content of the filler/binder or mixture of fillers/binders in the dosage form is within the range of 35±34 wt.-%, more preferably 35±28 wt.-%, still more preferably 35±22 wt.-%, yet more preferably 35±16 wt.-%, most preferably 35±10 wt.-%, and in particular 35±4 wt.-%, based on the total weight of dosage form. In another preferred embodiment, the total content of the filler/binder or mixture of fillers/binders in the dosage form is within the range of 40±39 wt.-%, more preferably 40±32 wt.-%, still more preferably 40±25 wt.-%, yet more preferably 40±18 wt.-%, most preferably 40±11 wt.-%, and in particular 40±4 wt.-%, based on the total weight of dosage form.

In a preferred embodiment, particularly when the dosage form is a capsule, the capsule is preferably filled with particle(s) A, which are optionally coated comprising portion b_{C} of the pharmacologically active ingredient b, and/or with portion b_{P} of the pharmacologically active ingredient b in form of a powder, and/or with optionally present particle(s) B, and/or with the optionally present granules comprising portion b_{G} of the pharmacologically active ingredient b; and additionally with a filler/binder, preferably lactose or mannitol.

In a preferred embodiment, the total content of the filler/binder is preferably within the range of 25±20 wt.-%, more preferably 25±15 wt.-%, still more preferably 25±10 wt.-%, and most preferably 25±5 wt.-%, based on the total weight of the dosage form. In another preferred embodiment, the total content of the filler/binder is preferably within the range of 35±30 wt.-%, more preferably 35±25 wt.-%, still more preferably 35±20 wt.-%, yet more preferably 35±15 wt.-%, even more preferably 35±10 wt.-%, and most preferably 35±5 wt.-%, based on the total weight of the dosage form. In still another preferred embodiment, the total content of the filler/binder is preferably within the range of 45±40 wt.-%, more preferably 45±35 wt.-%, still more preferably 45±30 wt.-%, yet more preferably 45±25 wt.-%, even more preferably 45±20 wt.-%, and most preferably 45±15 wt.-%, and in particular 45±10 wt.-%, based on the total weight of the dosage form. In yet another preferred embodiment, the total content of the filler/binder is preferably within the range of 55±40 wt.-%, more preferably 55±35 wt.-%, still more preferably 55±30 wt.-%, yet more preferably 55±25 wt.-%, even more preferably 55±20 wt.-%, and most preferably 55±15 wt.-%, and in particular 55±10 wt.-%, based on the total weight of the dosage form. In another preferred embodiment, the total content of the filler/binder is preferably within the range of 65±30 wt.-%, more preferably 65±25 wt.-%, still more preferably 65±20 wt.-%, yet more preferably 65±15 wt.-%, even more preferably 65±10 wt.-%, and most preferably 65±5 wt.-%, based on the total weight of the dosage form.

It has been surprisingly found that the filler/binder in the capsule filling can accelerate in vitro release of the pharmacologically active ingredient a and/or of the pharmacologically active ingredient b from the dosage form according to the invention.

Preferably, the filler/binder is contained in the optionally present particle(s) B but not in the particle(s) A of the dosage form according to the invention.

Preferably, the particle(s), the coating, the outer matrix material, the capsule filling, and/or the granules independently comprise a disintegrant, wherein the content of the disintegrant is more than 5.0 wt.-%, based on the total weight of the dosage form and/or based on the total weight of the particle(s), the coating, the outer matrix material, the capsule filling, and/or the granules, respectively.

In a preferred embodiment, particularly when the dosage form is a capsule, the dosage form contains the entire amount of disintegrant within the particle(s), i.e. outside the particle(s) there is preferably no disintegrant. Furthermore, the disintegrant is preferably homogeneously distributed in the particle(s). Preferably, when the particle(s) are coated, the coating does not contain disintegrant.

In another preferred embodiment, particularly when the dosage form is a tablet, the dosage form contains the disintegrant within the particle(s) as well as outside the particle(s). In a preferred embodiment, the nature of disintegrant within the particle(s) is identical with the nature of disintegrant outside the particle(s). However, different disintegrants inside the particle(s) and outside the particle(s) are also possible in accordance with the invention. Furthermore, the disintegrant is preferably homogeneously distributed in the particle(s). Preferably, when the particle(s) are coated, the coating does not contain disintegrant.

In still another preferred embodiment, particularly when the dosage form is the preferred tablet according to the invention, the dosage form contains the disintegrant outside the particle(s), and optionally also within the particle.

Suitable disintegrants are known to the skilled person and are preferably selected from the group consisting of polysaccharides, starches, starch derivatives, cellulose derivatives, polyvinylpyrrolidones, acrylates, and gas releasing substances. Croscarmellose is particularly preferred as disintegrant.

Preferred starches include but are not limited to “standard starch” (e.g. native maize starch) and pregelatinized starch (e.g. starch 1500).

Preferred starch derivatives include but are not limited to sodium starch glycolate (carboxymethyl starch sodium, e.g. Vivastar®).

Preferred cellulose derivatives include but are not limited to croscarmellose sodium (=crosslinked sodium carboxymethylcellulose; e.g. Vivasol®).

Preferred cellulose derivatives include but are not limited to croscarmellose sodium (=crosslinked sodium carboxymethylcellulose; e.g. Vivasol®), carmellose calcium (calcium carboxymethylcellulose), carmellose sodium (sodium carboxymethylcellulose), low substituted carmellose sodium (low substituted sodium carboxymethylcellulose; average degree of substitution (DS) 0.20 to 0.40, Mr 80,000 to 600,000 g/mol, CAS 9004-32-4, E 466), low substituted hydroxypropylcellulose (having a content of propyl groups within the range of from 5 to 16%; CAS 9004-64-2).

Preferred acrylates include but are not limited to carbopol.

Preferred polyvinylpyrrolidones include but are not limited to crospovidone (PVP

Preferred gas releasing substances include but are not limited to sodium bicarbonate.

Preferred disintegrants include but are not limited to crosslinked sodium carboxymethylcellulose (Na-CMC) (e.g. Crosscarmellose, Vivasol®, Ac-Di-S or); crosslinked casein (e.g. Esma-Spreng®); polysaccharide mixtures obtained from soybeans (e.g. Emcosoy®); maize starch or pretreated maize starch (e.g. Amijel®); alginic acid, sodium alginate, calcium alginate; polyvinylpyrrolidone (PVP) (e.g. Kollidone®, Polyplasdone®, Polydone®); crosslinked polyvinylpyrrolidone (PVP CI) (e.g. Polyplasdone® XL); starch and pretreated starch such as sodium carboxymethyl starch (=sodium starch glycolate, e.g. Explotab®, Prejel®, Primotab® ET, Starch® 1500, Ulmatryl®), and the mixtures thereof. Crosslinked polymers are particularly preferred disintegrants, especially crosslinked sodium carboxymethylcellulose (Na-CMC) or crosslinked polyvinylpyrrolidone (PVP CI).

Particularly preferred disintegrants are selected from the group consisting of
crosslinked sodium carboxymethylcellulose (Na-CMC) (e.g. Crosscarmellose, Vivasol®, Ac-Di-Sol®);
crosslinked casein (e.g. Esma-Spreng®);
alginic acid, sodium alginate, calcium alginate;
polysaccharide mixtures obtained from soybeans (e.g. Emcosoy®);
starch and pretreated starch such as sodium carboxymethyl starch (=sodium starch glycolate, e.g. Explotab®, Prejel®, Primotab® ET, Starch® 1500, Ulmatryl®);
maize starch or pretreated maize starch (e.g. Amijel®) ;
and mixtures of any of the foregoing.

Preferably, the content of the disintegrant is at least 6.0 wt.-%, at least 7.0 wt.-%, at least 8.0 wt.-%, at least 9.0 wt.-%, or at least 10 wt.-%, more preferably at least 12 wt.-%, still more preferably at least 14 wt.-%, yet more preferably at least 15 wt.-%, even more preferably at least 16 wt.-%, most preferably at least 18 wt.-%, and in particular at least 19 wt.-%, based on the total weight of the dosage form and/or based on the total weight of the particle(s), the coating, the outer matrix material, the capsule filling, and/or the granules, respectively.

It has been surprisingly found that the content of disintegrant typically has an optimum at which it provides the best balance of immediate release properties on the one hand and resistance against solvent extraction on the other hand. Said optimum may vary, but preferably is within the range of from about 10 wt.-% to about 20 wt.-%, relative to the total weight of the dosage form and/or based on the total weight of the particle(s).

In a preferred embodiment, the content of the disintegrant is within the range of 15±9.0 wt.-%, more preferably 15±8.5 wt.-%, still more preferably 15±8.0 wt.-%, yet more preferably 15±7.5 wt.-%, most preferably 15±7.0 wt.-%, and in particular 15±6.5 wt.-%, based on the total weight of the dosage form and/or based on the total weight of the particle(s), the coating, the outer matrix material, the capsule filling, and/or the granules, respectively. In still another preferred embodiment, the content of the disintegrant is within the range of 15±6.0 wt.-%, more preferably 15±5.5 wt.-%, still more preferably 15±5.0 wt.-%, yet more preferably 15±4.5 wt.-%, most preferably 15±4.0 wt.-%, and in particular 15±3.5 wt.-%, based on the total weight of the dosage form and/or based on the total weight of the particle(s), the coating, the outer matrix material, the capsule filling, and/or the granules, respectively. In another preferred embodiment, the content of the disintegrant is within the range of 15±3.0 wt.-%, more preferably 15±2.5 wt.-%, still more preferably 15±2.0 wt.-%, yet more preferably 15±1.5 wt.-%, most preferably 15±1.0 wt.-%, and in particular 15±0.5 wt.-%, based on the total weight of the dosage form and/or based on the total weight of the particle(s), the coating, the outer matrix material, the capsule filling, and/or the granules, respectively.

In another preferred embodiment, the content of the disintegrant is within the range of 20±15 wt.-% or 20±14 wt.-%, more preferably 20±13 wt.-%, still more preferably 20±12 wt.-%, yet more preferably 20±11 wt.-%, most preferably 20±10 wt.-%, and in particular 20±9.5 wt.-%, based on the total weight of the dosage form and/or based on the total weight of the particle(s), the coating, the outer matrix material, the capsule filling, and/or the granules, respectively. In another preferred embodiment, the content of the disintegrant is within the range of 20±9.0 wt.-%, more preferably 20±8.5 wt.-%, still more preferably 20±8.0 wt.-%, yet more preferably 20±7.5 wt.-%, most preferably 20±7.0 wt.-%, and in particular 20±6.5 wt.-%, based on the total weight of the dosage form and/or based on the total weight of the particle(s), the coating, the outer matrix material, the capsule filling, and/or the granules, respectively. In still another preferred embodiment, the content of the disintegrant is within the range of 20±6.0 wt.-%, more preferably 20±5.5 wt.-%, still more preferably 20±5.0 wt.-%, yet more preferably 20±4.5 wt.-%, most preferably 20±4.0 wt.-%, and in particular 20±3.5 wt.-%, based on the total weight of the dosage form and/or based on the total weight of the particle(s), the coating, the outer matrix material, the capsule filling, and/or the granules, respectively. In another preferred embodiment, the content of the disintegrant is within the range of 20±3.0 wt.-%, more preferably 20±2.5 wt.-%, still more preferably 20±2.0 wt.-%, yet more preferably 20±1.5 wt.-%, most preferably 20±1.0 wt.-%, and in particular 20±0.5 wt.-%, based on the total weight of the dosage form and/or based on the total weight of the particle(s), the coating, the outer matrix material, the capsule filling, and/or the granules, respectively.

In still another preferred embodiment, the content of the disintegrant is within the range of 25±9.0 wt.-%, more preferably 25±8.5 wt.-%, still more preferably 25±8.0 wt.-%, yet more preferably 25±7.5 wt.-%, most preferably 25±7.0 wt.-%, and in particular 25±6.5 wt.-%, based on the total weight of the dosage form and/or based on the total weight of the particle(s), the coating, the outer matrix material, the capsule filling, and/or the granules, respectively. In still another preferred embodiment, the content of the disintegrant is within the range of 25±6.0 wt.-%, more preferably 25±5.5 wt.-%, still more preferably 25±5.0 wt.-%, yet more preferably 25±4.5 wt.-%, most preferably 25±4.0 wt.-%, and in particular 25±3.5 wt.-%, based on the total weight of the dosage form and/or based on the total weight of the particle(s), the coating, the outer matrix material, the capsule filling, and/or the granules, respectively. In another preferred embodiment, the content of the disintegrant is within the range of 25±3.0 wt.-%, more preferably 25±2.5 wt.-%, still more preferably 25±2.0 wt.-%, yet more preferably 25±1.5 wt.-%, most preferably 25±1.0 wt.-%, and in particular 25±0.5 wt.-%, based on the total weight of the dosage form and/or based on the total weight of the particle(s), the coating, the outer matrix material, the capsule filling, and/or the granules respectively.

When the dosage form according to the invention contains more than a single disintegrant, e.g. a mixture of two different disintegrants, the above percentages preferably refer to the total content of disintegrants.

Preferably, the relative weight ratio of the polyalkylene oxide to the disintegrant is within the range of 8:1 to 1:5, more preferably 7:1 to 1:4, still more preferably 6:1 to 1:3, yet more preferably 5:1 to 1:2, most preferably 4:1 to 1:1, and in particular 3:1 to 2:1.

Preferably, the relative weight ratio of the pharmacologically active ingredient a to the disintegrant is within the range of 4:1 to 1:10, more preferably 3:1 to 1:9, still more preferably 2:1 to 1:8, yet more preferably 1:1 to 1:7, most preferably 1:2 to 1:6, and in particular 1:3 to 1:5.

The dosage form may contain a single disintegrant or a mixture of different disintegrants. Preferably, the dosage form contains a single disintegrant.

Preferably, the dosage form according to the invention and/or the particle(s), the coating, the outer matrix material, the capsule filling, and/or the granules independently additionally comprise a gelling agent, which is preferably a polysaccharide or a polyacrylate (acrylic polymer).

While the gelling agent may principally contribute to the overall resistance against solvent extraction of the dosage form according to the invention, it has been unexpectedly found that one or more disintegrants in comparatively high amounts in combination with one or more gelling agents are of particular advantage in this regard. It has been surprisingly found that the combination of one or more disintegrants in comparatively high amounts with one or more gelling agent is robust against variation of the pharmacologically active ingredient a and variation of the pharmacologically active ingredient b. Thus, according to the present invention exchanging a given pharmacologically active ingredient by another pharmacologically active ingredient does preferably not substantially alter the overall resistance against solvent extraction of the dosage form according to the invention

As used herein the term “gelling agent” is used to refer to a compound that, upon contact with a solvent (e.g. water), absorbs the solvent and swells, thereby forming a viscous or semi-viscous substance. Preferred gelling agents are not cross-linked. This substance may moderate release of the pharmacologically active ingredients in both aqueous and aqueous alcoholic media. Upon full hydration, a thick viscous solution or dispersion is typically produced that significantly reduces and/or minimizes the amount of free solvent which can contain an amount of solubilized pharmacologically active ingredient, and which can be drawn into a syringe. The gel that is formed may also reduce the overall amount of pharmacologically active ingredient extractable with the solvent by entrapping the pharmacologically active ingredient within a gel structure. Thus the gelling agent may play an important role in conferring tamper-resistance to the dosage forms according to the invention.

Gelling agents include pharmaceutically acceptable polymers, typically hydrophilic polymers, such as hydrogels. Representative examples of gelling agents include gums like xanthan gum, carrageenan, locust bean gum, guar, tragacanth, acaica (gum arabic), karaya, tara and gellan gum; polyethylene oxide, polyvinyl alcohol, hydroxypropylmethyl cellulose, carbomers, poly(uronic) acids and mixtures thereof.

Preferred gelling agents include acrylic polymers.

Thus, in a preferred embodiment, the polymer matrix of the particle(s) comprises a combination of a polyalkylene oxide and an acrylic polymer. Preferably, the relative weight ratio of the polyalkylene oxide to the acrylic polymer is within the range of from 10:1 to 1:6, more preferably 9:1 to 1:5, still more preferably 8:1 to 1:4, yet more preferably 7:1 to 1:3, even more preferably 6:1 to 1:2, most preferably 5:1 to 1:1, and in particular 4:1 to 2:1.

Preferred the acrylic polymer is an anionic polymer, i.e. derived from anionic acrylic monomers. Anionic acrylic monomers include but are not limited to
carboxylic acids, especially acrylic acid itself, methacrylic acid, ethacrylic acid, alpha-chloracrylic acid, alpha-cyano acrylic acid, beta-methyl-acrylic acid (crotonic acid), alpha-phenyl acrylic acid, beta-acryloxy propionic acid, sorbic acid, alpha-chloro sorbic acid, angelic acid, cinnamic acid, p-chloro cinnamic acid, beta-styryl acrylic acid (1-carboxy-4-phenyl butadiene-1,3), itaconic acid, citraconic acid, mesaconic acid, glutaconic acid, aconitic acid, maleic acid, fumaric acid, tricarboxy ethylene, maleic acid anhydride and the combinations thereof; and
sulfonic acids, especially aliphatic or aromatic vinyl sulfonic acids such as vinylsulfonic acid, allyl sulfonic acid, vinyltoluenesulfonic acid and styrene sulfonic acid; acrylic and methacrylic sulfonic acid such as sulfoethyl acrylate, sulfoethyl methacrylate, sulfopropyl acrylate, sulfopropyl methacrylate, 2-hydroxy-3-acryloxy propyl sulfonic acid, 2-hydroxy-3-methacryloxy propyl sulfonic acid and 2-acrylamido-2-methyl propane sulfonic acid.

Preferably, the anionic acrylic monomers are selected from the group consisting of acrylic acid, methacrylic acid, and/or 2-acrylamido-2-methyl propane sulfonic acid. Acrylic acid is especially preferred.

In a preferred embodiment, the acrylic polymer is cross-linked, i.e. is derived from a monomer composition comprising a cross-linking agent. Suitable cross-linking agents include
compounds having at least two polymerizable double bonds, e.g. ethylenically unsaturated functional groups;
compounds having at least one polymerizable double bond, e.g. an ethylenically unsaturated functional group, and at least one functional group that is capable of reacting with another functional group of one or more of the repeating units of acrylic polymer;
compounds having at least two functional groups that are capable of reacting with other functional groups of one or more of the repeating units of acrylic polymer; and
polyvalent metal compounds which can form ionic cross-linkages, e.g. through the anionic functional groups.

In a preferred embodiment, divinyl glycol (1,5-hexadiene-3,4-diol) is contained as cross-linking agent, whereas allyl or vinyl derivatives of polyols, such as allylsucrose or allyl pentaerythritol, are less preferred. This embodiment is preferably realized by polyacrylic acid polymers of polycarbophil type according to USP.

In another preferred embodiment, allyl derivatives of polyols, such as allylsucrose or allyl pentaerythritol, are contained as cross-linking agent, whereas divinyl glycol (1,5-hexadiene-3,4-diol) is less preferred. This embodiment is preferably realized by polyacrylic acid polymers of carbomer type according to USP or Ph. Eur.

In a preferred embodiment, acrylic polymer is a homopolymer of acrylic acid, optionally cross-linked, preferably with allyl sucrose or allyl pentaerythritol, in particular with allyl pentaerythritol. In another preferred embodiment, acrylic polymer is a copolymer of acrylic acid and C₁₀-C₃₀-alkyl acrylate, optionally cross-linked, preferably with allyl pentaerythritol. In another preferred embodiment, acrylic polymer is a so-called interpolymer, namely a homopolymer of acrylic acid, optionally cross-linked, preferably with allyl sucrose or allyl pentaerythritol; or a copolymer of acrylic acid and C₁₀-C₃₀-alkyl acrylate, optionally cross-linked, preferably with allyl pentaerythritol; which contain a block copolymer of polyethylene glycol and a long chain alkyl acid, preferably a C₈-C₃₀-alkyl acid. Polymers of this type are commercially available, e.g. under the trademark Carbopol®.

Preferably, the content of the gelling agent, preferably xanthan gum, is at least 1.0 wt.-%, more preferably at least 2.0 wt.-%, still more preferably at least 3.0 wt.-%, most preferably at least 4.0 wt.-%, based on the total weight of the dosage form and/or based on the total weight of the particle(s).

Preferably, the content of the gelling agent, preferably xanthan gum, is within the range of 5.0±4.5 wt.-%, more preferably 5.0±4.0 wt.-%, still more preferably 5.0±3.5 wt.-%, yet more preferably 5.0±3.0 wt.-%, even more preferably 5.0±2.5 wt.-%, most preferably 5.0±2.0 wt.-%, and in particular 5.0±1.5 wt.-%, based on the total weight of the dosage form and/or based on the total weight of the particle(s).

Preferably, the relative weight ratio of disintegrant:gelling agent is within the range of from 11:1 to 1:5, more preferably 10:1 to 1:4, still more preferably 9:1 to 1:3, yet more preferably 8:1 to 1:2, even more preferably 7:1 to 1:1, most preferably 6:1 to 2:1, and in particular 5:1 to 3:1.

Preferably, the dosage form according to the invention and/or the particle(s), the coating, the outer matrix material, the capsule filling, and/or the granules independently further comprise an antioxidant. Suitable antioxidants include ascorbic acid, butylated hydroxyanisole (BHA), butylated hydroxytoluene (BHT), salts of ascorbic acid, monothioglycerol, phosphorous acid, vitamin C, vitamin E and the derivatives thereof, coniferyl benzoate, nordihydroguajaretic acid, gallus acid esters, sodium bisulfate, particularly preferably butylhydroxytoluene or butylhydroxyanisole and α-tocopherol. The antioxidant is preferably present in quantities of 0.01 wt.-% to 10 wt.-%, more preferably of 0.03 wt.-% to 5 wt.-%, most preferably of 0.05 wt.-% to 2.5 wt.-%, based on the total weight of the dosage form and/or based on the total weight of the particle(s), the coating, the outer matrix material, the capsule filling, and/or the granules, respectively.

In a preferred embodiment, the dosage form according to the invention and/or the particle(s), the coating, the outer matrix material, the capsule filling, and/or the granules independently further comprise an acid, preferably citric acid. The amount of acid is preferably in the range of 0.01 wt.-% to 20 wt.-%, more preferably in the range of 0.02 wt.-% to 10 wt.-%, and still more preferably in the range of 0.05 wt.-% to 5 wt.-%, and most preferably in the range of 0.1 wt.-% to 1.0 wt.-%, based on the total weight of the dosage form and/or based on the total weight of the particle(s), the coating, the outer matrix material, the capsule filling, and/or the granules, respectively.

In a preferred embodiment, the dosage form according to the invention and/or the particle(s), the coating, the outer matrix material, the capsule filling, and/or the granules independently further comprise another polymer which is preferably selected from cellulose esters and cellulose ethers, in particular hydroxypropyl methylcellulose (HPMC).

The amount of the further polymer, preferably hydroxypropyl methylcellulose, preferably ranges from 0.1 wt.-% to 30 wt.-%, more preferably in the range of 1.0 wt.-% to 20 wt.-%, most preferably in the range of 2.0 wt.-% to 15 wt.-%, and in particular in the range of 3.5 wt.-% to 10.5 wt.-%, based on the total weight of the dosage form and/or based on the total weight of the particle(s), the coating, the outer matrix material, the capsule filling, and/or the granules, respectively.

When the polymer matrix of the particle(s) comprises polyalkylene oxide, in a preferred embodiment, the relative weight ratio of the polyalkylene oxide to the further polymer is within the range of 4.5±2:1, more preferably 4.5±1.5:1, still more preferably 4.5±1:1, yet more preferably 4.5±0.5:1, most preferably 4.5±0.2:1, and in particular 4.5±0.1:1. In another preferred embodiment, the relative weight ratio of the polyalkylene oxide to the further polymer is within the range of 8±7:1, more preferably 8±6:1, still more preferably 8±5:1, yet more preferably 8±4:1, most preferably 8±3:1, and in particular 8±2:1. In still another preferred embodiment, the relative weight ratio of the polyalkylene oxide to the further polymer is within the range of 11±8:1, more preferably 11±7:1, still more preferably 11±6:1, yet more preferably 11±5:1, most preferably 11±4:1, and in particular 11±3:1.

In another preferred embodiment, the dosage form and/or the particle(s) according to the invention do not contain any further polymer besides the polyalkylene oxide and optionally, polyethylene glycol.

In a preferred embodiment, the dosage form according to the invention contains at least one lubricant. Preferably, the lubricant is contained in the dosage form outside the particle(s), i.e. the particle(s) as such preferably do not contain lubricant. The lubricant can be independently contained in the coating, the outer matrix material, and/or the granules.

Especially preferred lubricants are selected from
magnesium stearate and stearic acid;
glycerides of fatty acids, including monoglycerides, diglycerides, triglycerides, and mixtures thereof; preferably of C₆ to C₂₂ fatty acids; especially preferred are partial glycerides of the C₁₆ to C₂₂ fatty acids such as glycerol behenat, glycerol palmitostearate and glycerol monostearate;
polyoxyethylene glycerol fatty acid esters, such as mixtures of mono-, di- and triesters of glycerol and di- and monoesters of macrogols having molecular weights within the range of from 200 to 4000 g/mol, e.g., macrogolglycerolcaprylocaprate, macrogolglycerollaurate, macrogolglycerolococoate, macrogolglycerollinoleate, macrogol-20-glycerolmonostearate, macrogol-6-glycerolcaprylocaprate, macrogolglycerololeate; macrogolglycerolstearate, macrogolglycerolhydroxystearate, and macrogolglycerolrizinoleate;
polyglycolyzed glycerides, such as the one known and commercially available under the trade name “Labrasol”;
fatty alcohols that may be linear or branched, such as cetylalcohol, stearylalcohol, cetylstearyl alcohol, 2-octyldodecane-1-ol and 2-hexyldecane-1-ol;
polyethylene glycols having a molecular weight between 10.000 and 60.000 g/mol; and
natural semi-synthetic or synthetic waxes, preferably waxes with a softening point of at least 50° C., more preferably 60° C., and in particular carnauba wax and bees wax.

Preferably, the amount of the lubricant ranges from 0.01 wt.-% to 10 wt.-%, more preferably in the range of 0.05 wt.-% to 7.5 wt.-%, most preferably in the range of 0.1 wt.-% to 5 wt.-%, and in particular in the range of 0.1 wt.-% to 1 wt.-%, based on the total weight of the dosage form and/or based on the total weight of the particle(s), the coating, the outer matrix material, the capsule filling, and/or the granules, respectively.

In another preferred embodiment, the dosage form contains no lubricant.

Preferably, the dosage form according to the invention and/or the particle(s), the coating, the outer matrix material, the capsule filling, and/or the granules independently further comprise a plasticizer. The plasticizer improves the processability of the polymer matrix that preferably comprises polyalkylene oxide. A preferred plasticizer is polyalkylene glycol, like polyethylene glycol, triacetin, fatty acids, fatty acid esters, waxes and/or microcrystalline waxes. Particularly preferred plasticizers are polyethylene glycols, such as PEG 6000 (Macrogol 6000).

Preferably, the content of the plasticizer is within the range of from 0.5 to 30 wt.-%, more preferably 1.0 to 25 wt.-%, still more preferably 2.5 wt.-% to 22.5 wt.-%, yet more preferably 5.0 wt.-% to 20 wt.-%, most preferably 6 to 20 wt.-% and in particular 7 wt.-% to 17.5 wt.-%, based on the total weight of the dosage form and/or based on the total weight of the particle(s), the coating, the outer matrix material, the capsule filling, and/or the granules, respectively.

In a preferred embodiment, the plasticizer is a polyalkylene glycol having a content within the range of 7±6 wt.-%, more preferably 7±5 wt.-%, still more preferably 7±4 wt.-%, yet more preferably 7±3 wt.-%, most preferably 7±2 wt.-%, and in particular 7±1 wt.-%, based on the total weight of the dosage form and/or based on the total weight of the particle(s), the coating, the outer matrix material, the capsule filling, and/or the granules, respectively. In another preferred embodiment, the plasticizer is a polyalkylene glycol having a content within the range of 10±8 wt.-%, more preferably 10±6 wt.-%, still more preferably 10±5 wt.-%, yet more preferably 10±4 wt.-%, most preferably 10±3 wt.-%, and in particular 10±2 wt.-%, based on the total weight of the dosage form and/or based on the total weight of the particle(s), the coating, the outer matrix material, the capsule filling, and/or the granules, respectively.

In a preferred embodiment, the relative weight ratio of the polyalkylene oxide to the polyalkylene glycol is within the range of 5.4±2:1, more preferably 5.4±1.5:1, still more preferably 5.4±1:1, yet more preferably 5.4±0.5:1, most preferably 5.4±0.2:1, and in particular 5.4±0.1:1. This ratio satisfies the requirements of relative high polyalkylene oxide content and good extrudability.

Plasticizers can sometimes act as a lubricant, and lubricants can sometimes act as a plasticizer.

In a preferred embodiment, particularly when the particle(s) contain at least a portion b_{A} or b_{B} of the pharmacologically active ingredient b, particularly acetaminophen, a plasticizer can be omitted. It has been surprisingly found that acetaminophen among other pharmacologically active ingredients b can act as plasticizer e.g. in hot-melt extrusion technology.

In preferred compositions of the particle(s) A that are preferably hot-melt extruded and that are contained in the dosage form according to the invention, the polymer matrix comprises a polyalkylene oxide, preferably a polyethylene oxide with a weight average molecular weight within the range of from 0.5 to 15 million g/mol.

When the particle(s) A comprise pharmacologically active ingredient a but no pharmacologically active ingredient b, particularly preferred embodiments C¹ to C¹² are summarized in the tables here below:

| per particle A [wt.-%] | C¹ | C² | C³ | C⁴ |
|---|---|---|---|---|
| pharmacologically active | 5.50 ± 5.00 | 5.50 ± 4.00 | 5.50 ± 3.00 | 5.50 ± 2.00 |
| ingredient a | | | | |
| polyalkylene oxide | 60.00 ± 35.00 | 60.00 ± 30.00 | 60.00 ± 25.00 | 60.00 ± 15.00 |
| optionally acid, e.g. citric acid | 0.80 ± 0.75 | 0.80 ± 0.65 | 0.80 ± 0.50 | 0.80 ± 0.35 |
| optionally plasticizer, e.g. | 14.00 ± 13.50 | 14.00 ± 10.00 | 14.00 ± 7.50 | 14.00 ± 5.00 |
| polyethylene glycol | | | | |
| optionally antioxidant, e.g. α- | 0.20 ± 0.18 | 0.20 ± 0.14 | 0.20 ± 0.10 | 0.20 ± 0.06 |
| tocopherol | | | | |
| optionally crosslinked polyacrylic | 20.00 ± 19.50 | 20.00 ± 15.00 | 20.00 ± 10.00 | 20.00 ± 5.00 |
| acid, e.g. Carbopol 71G | | | | |
| per particle A [wt.-%] | C⁵ | C⁶ | C⁷ | C⁸ |
| pharmacologically active | 15.00 ± 25.00 | 15.00 ± 20.00 | 15.00 ± 15.00 | 15.00 ± 10.00 |
| ingredient a | | | | |
| polyalkylene oxide | 60.00 ± 35.00 | 60.00 ± 30.00 | 60.00 ± 25.00 | 60.00 ± 15.00 |
| optionally acid, e.g. citric acid | 0.80 ± 0.75 | 0.80 ± 0.65 | 0.80 ± 0.50 | 0.80 ± 0.35 |
| optionally plasticizer, e.g. | 11.00 ± 8.00 | 11.00 ± 6.00 | 11.00 ± 5.00 | 11.00 ± 4.00 |
| polyethylene glycol | | | | |
| optionally antioxidant, e.g. α- | 0.20 ± 0.18 | 0.20 ± 0.14 | 0.20 ± 0.10 | 0.20 ± 0.06 |
| tocopherol | | | | |
| optionally crosslinked polyacrylic | 20.00 ± 19.50 | 20.00 ± 15.00 | 20.00 ± 10.00 | 20.00 ± 5.00 |
| acid, e.g. Carbopol 71G | | | | |
| per particle A [wt.-%] | C⁹ | C¹⁰ | C¹¹ | C¹² |
| pharmacologically active | 30.00 ± 25.00 | 30.00 ± 20.00 | 30.00 ± 15.00 | 30.00 ± 10.00 |
| ingredient a | | | | |
| polyalkylene oxide | 60.00 ± 35.00 | 60.00 ± 30.00 | 60.00 ± 25.00 | 60.00 ± 15.00 |
| optionally acid, e.g. citric acid | 0.80 ± 0.75 | 0.80 ± 0.65 | 0.80 ± 0.50 | 0.80 ± 0.35 |
| optionally plasticizer, e.g. | 9.00 ± 8.00 | 9.00 ± 6.00 | 9.00 ± 5.00 | 9.00 ± 4.00 |
| polyethylene glycol | | | | |
| optionally antioxidant, e.g. α- | 0.20 ± 0.18 | 0.20 ± 0.14 | 0.20 ± 0.10 | 0.20 ± 0.06 |
| tocopherol | | | | |
| optionally crosslinked polyacrylic | 20.00 ± 19.50 | 20.00 ± 15.00 | 20.00 ± 10.00 | 20.00 ± 5.00 |
| acid, e.g. Carbopol 71G | | | | |

(all percentages relative to the total weight of the particle(s) A).

When the particle(s) A comprise pharmacologically active ingredient a as well as pharmacologically active ingredient b, particularly preferred embodiments D¹ to D⁴ are summarized in the table here below:

| per particle A [wt.-%] | D¹ | D² | D³ | D⁴ |
|---|---|---|---|---|
| pharmacologically active | 5.50 ± 5.00 | 5.50 ± 4.00 | 5.50 ± 3.00 | 5.50 ± 2.00 |
| ingredient a | | | | |
| polyalkylene oxide | 55.00 ± 40.00 | 55.00 ± 35.00 | 55.00 ± 25.00 | 55.00 ± 15.00 |
| optionally pharmacologically | 10.00 ± 9.50 | 10.00 ± 8.00 | 10.00 ± 5.00 | 10.00 ± 2.00 |
| active ingredient b | | | | |
| optionally acid, e.g. citric acid | 0.80 ± 0.75 | 0.80 ± 0.65 | 0.80 ± 0.50 | 0.80 ± 0.35 |
| optionally plasticizer, e.g. | 14.00 ± 13.50 | 14.00 ± 10.00 | 14.00 ± 7.50 | 14.00 ± 5.00 |
| polyethylene glycol | | | | |
| optionally antioxidant, e.g. α- | 0.20 ± 0.18 | 0.20 ± 0.14 | 0.20 ± 0.10 | 0.20 ± 0.06 |
| tocopherol | | | | |
| optionally crosslinked polyacrylic | 17.00 ± 16.50 | 17.00 ± 15.00 | 17.00 ± 10.00 | 17.00 ± 5.00 |
| acid, e.g. Carbopol 71G | | | | |
| optionally gelling agent, e.g. | 5.00 ± 4.50 | 5.00 ± 3.50 | 5.00 ± 2.50 | 5.00 ± 1.50 |
| xanthan | | | | |

(all percentages relative to the total weight of the particle(s) A).

In the above tables, “optionally” in the context of the pharmacologically active ingredient b, the acid, the plasticizer, the antioxidant, the crosslinked polyacrylic acid, and the gelling agent means that these excipients may independently of one another be contained in the particle(s) A or not, and provided that they are contained in the particle(s) A, their content in wt.-% is as specified.

In preferred compositions of the optionally present particle(s) B that are preferably also hot-melt extruded and that are contained in the dosage form according to the invention, the polymer matrix comprises a polyalkylene oxide, preferably a polyethylene oxide with a weight average molecular weight within the range of from 0.5 to 15 million g/mol. Particularly preferred embodiments E¹ to E¹² are summarized in the tables here below:

| per particle B [wt.-%] | E¹ | E² | E³ | E⁴ |
|---|---|---|---|---|
| pharmacologically active | 10.00 ± 9.50 | 10.00 ± 8.00 | 10.00 ± 5.00 | 10.00 ± 2.00 |
| ingredient b | | | | |
| polyalkylene oxide | 62.00 ± 38.00 | 62.00 ± 30.00 | 62.00 ± 25.00 | 62.00 ± 15.00 |
| optionally acid, e.g. citric acid | 0.80 ± 0.75 | 0.80 ± 0.65 | 0.80 ± 0.50 | 0.80 ± 0.35 |
| optionally plasticizer, e.g. | 10.00 ± 9.50 | 10.00 ± 7.50 | 10.00 ± 5.00 | 10.00 ± 2.50 |
| polyethylene glycol | | | | |
| optionally antioxidant, e.g. α- | 0.20 ± 0.18 | 0.20 ± 0.14 | 0.20 ± 0.10 | 0.20 ± 0.06 |
| tocopherol | | | | |
| optionally crosslinked polyacrylic | 17.00 ± 16.50 | 17.00 ± 15.00 | 17.00 ± 10.00 | 17.00 ± 5.00 |
| acid, e.g. Carbopol 71G | | | | |
| per particle B [wt.-%] | E⁵ | E⁶ | E⁷ | E⁸ |
| pharmacologically active | 20.00 ± 16.00 | 20.00 ± 12.00 | 20.00 ± 8.00 | 20.00 ± 4.00 |
| ingredient b | | | | |
| polyalkylene oxide | 62.00 ± 38.00 | 62.00 ± 30.00 | 62.00 ± 25.00 | 62.00 ± 15.00 |
| optionally acid, e.g. citric acid | 0.80 ± 0.75 | 0.80 ± 0.65 | 0.80 ± 0.50 | 0.80 ± 0.35 |
| optionally plasticizer, e.g. | 10.00 ± 9.50 | 10.00 ± 7.50 | 10.00 ± 5.00 | 10.00 ± 2.50 |
| polyethylene glycol | | | | |
| optionally antioxidant, e.g. α- | 0.20 ± 0.18 | 0.20 ± 0.14 | 0.20 ± 0.10 | 0.20 ± 0.06 |
| tocopherol | | | | |
| optionally crosslinked polyacrylic | 17.00 ± 16.50 | 17.00 ± 15.00 | 17.00 ± 10.00 | 17.00 ± 5.00 |
| acid, e.g. Carbopol 71G | | | | |
| per particle B [wt.-%] | E⁹ | E¹⁰ | E¹¹ | E¹² |
| pharmacologically active | 30.00 ± 25.00 | 30.00 ± 20.00 | 30.00 ± 15.00 | 30.00 ± 10.00 |
| ingredient b | | | | |
| polyalkylene oxide | 62.00 ± 38.00 | 62.00 ± 30.00 | 62.00 ± 25.00 | 62.00 ± 15.00 |
| optionally acid, e.g. citric acid | 0.80 ± 0.75 | 0.80 ± 0.65 | 0.80 ± 0.50 | 0.80 ± 0.35 |
| optionally plasticizer, e.g. | 10.00 ± 9.50 | 10.00 ± 7.50 | 10.00 ± 5.00 | 10.00 ± 2.50 |
| polyethylene glycol | | | | |
| optionally antioxidant, e.g. α- | 0.20 ± 0.18 | 0.20 ± 0.14 | 0.20 ± 0.10 | 0.20 ± 0.06 |
| tocopherol | | | | |
| optionally crosslinked polyacrylic | 17.00 ± 16.50 | 17.00 ± 15.00 | 17.00 ± 10.00 | 17.00 ± 5.00 |
| acid, e.g. Carbopol 71G | | | | |

(all percentages relative to the total weight of the particle(s) B).

In the above tables, “optionally” in the context of the acid, the plasticizer, the antioxidant, and the crosslinked polyacrylic acid means that these excipients may independently of one another be contained in the particle(s) B or not, and provided that they are contained in the particle(s) B, their content in wt.-% is as specified.

In preferred embodiments, particle(s) A and/or particle(s) B comprise a coating comprising at least a portion b_{C} of the pharmacologically active ingredient b. Particularly preferred embodiments of the coating composition F¹ to F⁴ are summarized in the table here below:

| per coating [wt.-%] | F¹ | F² | F³ | F⁴ |
|---|---|---|---|---|
| pharmacologically active | 20.00 ± 19.50 | 20.00 ± 18.00 | 20.00 ± 12.00 | 20.00 ± 6.00 |
| ingredient b | | | | |
| film forming polymer, e.g. based | 30.00 ± 29.00 | 30.00 ± 25.00 | 30.00 ± 20.00 | 30.00 ± 15.00 |
| on PVA or HPMC | | | | |
| optionally, plasticizer | 10.00 ± 9.50 | 10.00 ± 7.50 | 10.00 ± 5.00 | 10.00 ± 2.50 |
| optionally, further excipients, e.g. | 5.00 ± 4.50 | 5.00 ± 4.00 | 5.00 ± 3.00 | 5.00 ± 2.00 |
| anti-tacking, dyes, antioxidants | | | | |

(all percentages relative to the total weight of the granules).

Particularly preferred embodiments G¹ to G⁴ of the granules according to the invention are summarized in the table here below:

| per granule [wt.-%] | G¹ | G² | G³ | G⁴ |
|---|---|---|---|---|
| pharmacologically active | 62.00 ± 35.00 | 62.00 ± 30.00 | 62.00 ± 25.00 | 62.00 ± 15.00 |
| ingredient b | | | | |
| filler/binder, e.g. microcrystalline | 30.00 ± 29.00 | 30.00 ± 25.00 | 30.00 ± 20.00 | 30.00 ± 15.00 |
| cellulose | | | | |
| optionally, disintegrant, | 6.00 ± 5.50 | 6.00 ± 5.50 | 6.00 ± 5.50 | 6.00 ± 5.50 |
| e.g. croscarmellose sodium | | | | |
| optionally, further polymer, e.g. | 1.50 ± 1.40 | 1.50 ± 1.20 | 1.50 ± 1.00 | 1.50 ± 0.80 |
| hypromellose | | | | |

(all percentages relative to the total weight of the granules).

In the above table, “optionally” in the context of the disintegrant and the further polymer means that these excipients may independently of one another be contained in the granules or not, and provided that they are contained in the granules, their content in wt.-% is as specified.

In a preferred embodiment of the dosage form according to the invention, the particle(s) A and/or the optionally present particle(s) B are hot melt-extruded. Thus, the particle(s) according to the invention are preferably prepared by melt-extrusion, although also other methods of thermoforming may be used in order to manufacture the particle(s) according to the invention such as press-molding at elevated temperature or heating of particle(s) that were manufactured by conventional compression in a first step and then heated above the softening temperature of the polyalkylene oxide in the particle(s) in a second step to form hard dosage forms. In this regards, thermoforming means the forming, or molding of a mass after the application of heat. In a preferred embodiment, the particle(s) are thermoformed by hot-melt extrusion.

In a preferred embodiment, the particle(s) are prepared by hot melt-extrusion, preferably by means of a twin-screw-extruder. Melt extrusion preferably provides a melt-extruded strand that is preferably cut into monoliths, which are then optionally compressed and formed into particle(s). Preferably, compression is achieved by means of a die and a punch, preferably from a monolithic mass obtained by melt extrusion. If obtained via melt extrusion, the compressing step is preferably carried out with a monolithic mass exhibiting ambient temperature, that is, a temperature in the range from 20 to 25° C. The strands obtained by way of extrusion can either be subjected to the compression step as such or can be cut prior to the compression step. This cutting can be performed by usual techniques, for example using rotating knives or compressed air, at elevated temperature, e.g. when the extruded stand is still warm due to hot-melt extrusion, or at ambient temperature, i.e. after the extruded strand has been allowed to cool down. When the extruded strand is still warm, singulation of the extruded strand into extruded particle(s) is preferably performed by cutting the extruded strand immediately after it has exited the extrusion die. It is possible to subject the extruded strands to the compression step or to the cutting step when still warm, that is more or less immediately after the extrusion step. The extrusion is preferably carried out by means of a twin-screw extruder.

The particle(s) of the dosage form according to the invention may be produced by different processes, the particularly preferred of which are explained in greater detail below. Several suitable processes have already been described in the prior art. In this regard it can be referred to, e.g., WO 2005/016313, WO 2005/016314, WO 2005/063214, WO 2005/102286, WO 2006/002883, WO 2006/002884, WO 2006/002886, WO 2006/082097, and WO 2006/082099.

In general, the process for the production of the particle(s) according to the invention preferably comprises the following steps:
(a) mixing all ingredients;
(b) optionally pre-forming the mixture obtained from step (a), preferably by applying heat and/or force to the mixture obtained from step (a), the quantity of heat supplied preferably not being sufficient to heat the polyalkylene oxide up to its softening point;
(c) hardening the mixture by applying heat and force, it being possible to supply the heat during and/or before the application of force and the quantity of heat supplied being sufficient to heat the polyalkylene oxide at least up to its softening point; and thereafter allowing the material to cool and removing the force
(d) optionally singulating the hardened mixture; and
(e) optionally providing a film coating.

In a preferred embodiment, the mixture of ingredients is heated and subsequently compressed under conditions (time, temperature and pressure) sufficient in order to achieve the desired mechanical properties, e.g. in terms of breaking strength and the like. This technique may be achieved e.g. by means of a tabletting tool which is either heated and/or which is filled with the heated mixture that is subsequently compressed without further supply of heat or with simultaneous additional supply of heat.

In another preferred embodiment, the mixture of ingredients is heated and simultaneously compressed under conditions (time, temperature and pressure) sufficient in order to achieve the desired mechanical properties, e.g. in terms of breaking strength and the like. This technique may be achieved e.g. by means of an extruder with one or more heating zones, wherein the mixture is heated and simultaneously subjected to extrusion forces finally resulting in a compression of the heated mixture.

In still another embodiment, the mixture of ingredients is compressed under ambient conditions at sufficient pressure and subsequently heated (cured) under conditions (time, temperature) sufficient in order to achieve the desired mechanical properties, e.g. in terms of breaking strength and the like. This technique may be achieved e.g. by means of a curing oven in which the compressed articles are cured for a sufficient time at a sufficient temperature, preferably without exerting any further pressure. Such process is further described e.g. in US 2009/0081290.

Heat may be supplied directly, e.g. by contact or by means of hot gas such as hot air, or with the assistance of ultrasound; or is indirectly supplied by friction and/or shear. Force may be applied and/or the particle(s) may be shaped for example by direct tabletting or with the assistance of a suitable extruder, particularly by means of a screw extruder equipped with one or two screws (single-screw-extruder and twin-screw-extruder, respectively) or by means of a planetary gear extruder.

The final shape of the particle(s) may either be provided during the hardening of the mixture by applying heat and force (step (c)) or in a subsequent step (step (e)). In both cases, the mixture of all components is preferably in the plastified state, i.e. preferably, shaping is performed at a temperature at least above the softening point of the polyalkylene oxide. However, extrusion at lower temperatures, e.g. ambient temperature, is also possible and may be preferred.

A particularly preferred process for the manufacture of the particle(s) according to the invention involves hot-melt extrusion. In this process, the particle(s) according to the invention are produced by thermoforming with the assistance of an extruder, preferably without there being any observable consequent discoloration of the extrudate.

This process is characterized in that
a) all components are mixed,
b) the resultant mixture is heated in the extruder at least up to the softening point of the polyalkylene oxide and extruded through the outlet orifice of the extruder by application of force,
c) the still plastic extrudate is singulated and formed into the particle(s) or
d) the cooled and optionally reheated singulated extrudate is formed into the particle(s).

Mixing of the components according to process step a) may also proceed in the extruder.

The components may also be mixed in a mixer known to the person skilled in the art. The mixer may, for example, be a roll mixer, shaking mixer, shear mixer or compulsory mixer.

The, preferably molten, mixture which has been heated in the extruder at least up to the softening point of polyalkylene oxide is extruded from the extruder through a die with at least one bore, preferably a multitude of bores.

The process according to the invention requires the use of suitable extruders, preferably screw extruders. Screw extruders which are equipped with two screws (twin-screw-extruders) are particularly preferred.

Preferably, extrusion is performed in the absence of water, i.e., no water is added. However, traces of water (e.g., caused by atmospheric humidity) may be present.

The extruder preferably comprises at least two temperature zones, with heating of the mixture at least up to the softening point of the polyalkylene oxide proceeding in the first zone, which is downstream from a feed zone and optionally mixing zone. The throughput of the mixture is preferably from 1.0 kg to 15 kg/hour. In a preferred embodiment, the throughput is from 0.5 kg/hour to 3.5 kg/hour. In another preferred embodiment, the throughput is from 4 to 15 kg/hour.

In a preferred embodiment, the die head pressure is within the range of from 25 to 200 bar. The die head pressure can be adjusted inter alia by die geometry, temperature profile, extrusion speed, number of bores in the dies, screw configuration, first feeding steps in the extruder, and the like.

The die geometry or the geometry of the bores is freely selectable. The die or the bores may accordingly exhibit a round, oblong or oval cross-section, wherein the round cross-section preferably has a diameter of 0.1 mm to 2 mm, preferably of 0.5 mm to 0.9 mm. Preferably, the die or the bores have a round cross-section. The casing of the extruder used according to the invention may be heated or cooled. The corresponding temperature control, i.e. heating or cooling, is so arranged that the mixture to be extruded exhibits at least an average temperature (product temperature) corresponding to the softening temperature of the polyalkylene oxide and does not rise above a temperature at which the pharmacologically active ingredient a to be processed may be damaged. Preferably, the temperature of the mixture to be extruded is adjusted to below 180° C., preferably below 150° C., but at least to the softening temperature of polyalkylene oxide. Typical extrusion temperatures are 120° C. and 150° C.

In a preferred embodiment, the extruder torque is within the range of from 30 to 95%. Extruder torque can be adjusted inter alia by die geometry, temperature profile, extrusion speed, number of bores in the dies, screw configuration, first feeding steps in the extruder, and the like.

After extrusion of the molten mixture and optional cooling of the extruded strand or extruded strands, the extrudates are preferably singulated. This singulation may preferably be performed by cutting up the extrudates by means of revolving or rotating knives, wires, blades or with the assistance of laser cutters.

Preferably, intermediate or final storage of the optionally singulated extrudate or the final shape of the particle(s) according to the invention is performed under oxygen-free atmosphere which may be achieved, e.g., by means of oxygen-scavengers.

The singulated extrudate may be press-formed into particle(s) in order to impart the final shape to the particle(s).

The application of force in the extruder onto the at least plasticized mixture is adjusted by controlling the rotational speed of the conveying device in the extruder and the geometry thereof and by dimensioning the outlet orifice in such a manner that the pressure necessary for extruding the plasticized mixture is built up in the extruder, preferably immediately prior to extrusion. The extrusion parameters which, for each particular composition, are necessary to give rise to a dosage form with desired mechanical properties, may be established by simple preliminary testing.

For example but not limiting, extrusion may be performed by means of a twin-screw-extruder type ZSE 18 or ZSE27 (Leistritz, Nürnberg, Germany), screw diameters of 18 or 27 mm. Screws having eccentric or blunt ends may be used. A heatable die with a round bore or with a multitude of bores each having a diameter of 0.2, 0.3, 0.4, 0.5, 0.6, 0.7, 0.8, 0.9 or 1.0 mm may be used. For a twin-screw-extruder type ZSE 18, the extrusion parameters may be adjusted e.g. to the following values: rotational speed of the screws: 120 Upm; delivery rate 2 kg/h for a ZSE 18 or 5 kg/h, 10 kg/h, or even 20 kg/h and more for a ZSE27; product temperature: in front of die 125° C. and behind die 135° C.; and jacket temperature: 110° C. The throughput can generally be increased by increasing the number of dies at the extruder outlet.

Preferably, extrusion is performed by means of twin-screw-extruders or planetary-gear-extruders, twin-screw extruders (co-rotating or contra-rotating) being particularly preferred.

The particle(s) according to the invention are preferably produced by thermoforming with the assistance of an extruder without any observable consequent discoloration of the extrudates. The particle(s) may be produced e.g. by means of a Micro Pelletizer (Leistritz, Nürnberg, Germany).

The process for the preparation of the particle(s) according to the invention is preferably performed continuously. Preferably, the process involves the extrusion of a homogeneous mixture of all components. It is particularly advantageous if the thus obtained intermediate, e.g. the strand obtained by extrusion, exhibits uniform properties. Particularly desirable are uniform density, uniform distribution of the active compound, uniform mechanical properties, uniform porosity, uniform appearance of the surface, etc. Only under these circumstances the uniformity of the pharmacological properties, such as the stability of the release profile, may be ensured and the amount of rejects can be kept low.

Preferably, the particle(s) according to the invention can be regarded as “extruded pellets”. The term “extruded pellets” has structural implications which are understood by persons skilled in the art. A person skilled in the art knows that pelletized dosage forms can be prepared by a number of techniques, including:
drug layering on nonpareil sugar or microcrystalline cellulose beads,
spray drying,
spray congealing,
rotogranulation,
hot-melt extrusion,
spheronization of low melting materials, or
extrusion-spheronization of a wet mass.

Accordingly, “extruded pellets” can be obtained either by hot-melt extrusion or by extrusion-spheronization.

“Extruded pellets” can be distinguished from other types of pellets, as extruded pellets typically have a different shape. The shape of the extruded pellets is typically more cut-rod-like than perfectly globated round.

“Extruded pellets” can be distinguished from other types of pellets because they are structurally different. For example, drug layering on nonpareils yields multilayered pellets having a core, whereas extrusion typically yields a monolithic mass comprising a homogeneous mixture of all ingredients. Similarly, spray drying and spray congealing typically yield spheres, whereas extrusion typically yields cylindrical extrudates which can be subsequently spheronized.

The structural differences between “extruded pellets” and “agglomerated pellets” are significant because they may affect the release of active substances from the pellets and consequently result in different pharmacological profiles. Therefore, a person skilled in the pharmaceutical formulation art would not consider “extruded pellets” to be equivalent to “agglomerated pellets”.

When the coating of particle(s) A comprises the total amount of the pharmacologically active ingredient b or portion b_{C} thereof, said coating may be applied to particle(s) A by conventional means such as spray coating, dip coating, in a fluidized bed and the like. Suitable methods and apparatuses are known to the skilled person.

For that purpose, all constituents of the coating are preferably mixed with one another, optionally with one or more solvents, and then applied on the particle(s) A. When the mixtures contain one or more solvents, the application preferably proceeds under evaporative conditions.

When the granules according to the invention comprise the total amount of the pharmacologically active ingredient b or portion b_{C} thereof, said granules are preferably manufactured by wet granulation techniques or by dry granulation techniques. Suitable methods and apparatuses are known to the skilled person.

In a preferred embodiment, the granules are manufactured by a wet granulation process, preferably in a one-pot granulator. Preferred granulation solvents include but are not limited to water, ethanol and the mixtures thereof. Preferably, granulation is achieved in a fluidized bed granulator. Alternatively, granulation may be achieved by wet extrusion.

In another preferred embodiment, the granules are manufactured by dry granulation, optionally followed by roller compaction.

The dosage forms according to the invention may be prepared by any conventional method. Suitable methods and apparatuses are known to the skilled person.

When the dosage form is a capsule, all components may be filled separately or as admixture into the capsules. Said components may include but are not limited to particle(s) A, which may optionally be provided with a coating comprising pharmacologically active ingredient b or portion b_{C} thereof, the optionally present particle(s) B, the optionally present powder of pharmacologically active ingredient b, and the optionally present granules of pharmacologically active ingredient b, respectively.

When the dosage form is a tablet, the tablet is preferably prepared by compression. Thus, particle(s) are preferably mixed, e.g. blended and/or granulated (e.g. wet granulated), e.g. with matrix material of the preferred table according to the invention, the optionally present powder of pharmacologically active ingredient b and the optionally present granules of pharmacologically active ingredient b, respectively, and the resulting mix (e.g. blend or granulate) is then compressed, preferably in moulds, to form dosage forms. It is also envisaged that the particle(s) may be incorporated into a matrix using other processes, such as by melt granulation (e.g. using fatty alcohols and/or water-soluble waxes and/or water-insoluble waxes) or high shear granulation, followed by compression.

When the dosage forms according to the invention are manufactured by means of an eccentric press, the compression force is preferably within the range of from 5 to 30 kN, preferably from 15 to 25 kN. When the dosage forms according to the invention are manufactured by means of a rotating press, the compression force is preferably within the range of from 5 to 40 kN, in certain embodiments >25 kN, in other embodiments 13 kN.

The particle(s) A and dosage forms according to the invention may be used in medicine, e.g. as an analgesic. The particle(s) A and dosage forms are therefore particularly suitable for the treatment or management of pain. In such dosage forms, the pharmacologically active ingredient a is preferably an analgesic.

A further aspect according to the invention relates to the dosage form as described above for use in the treatment of pain. A further aspect of the invention relates to the use of a pharmacologically active ingredient a and/or of a pharmacologically active ingredient b for the manufacture of a dosage form according to the invention for the treatment of pain. A further aspect of the invention relates to a method for the treatment of pain comprising the administration, preferably oral administration of a dosage form according to the invention to a subject in need thereof.

A further aspect according to the invention relates to the use of a dosage form according to the invention for avoiding or hindering the abuse of the pharmacologically active ingredient a and optionally also of the pharmacologically active ingredient b contained therein.

A further aspect according to the invention relates to the use of a dosage form according to the invention for avoiding or hindering the unintentional overdose of the pharmacologically active ingredient a contained therein.

In this regard, the invention also relates to the use of a pharmacologically active ingredient a and/or of a pharmacologically active ingredient b for the manufacture of the dosage form according to the invention for the prophylaxis and/or the treatment of a disorder, thereby preventing an overdose of the pharmacologically active ingredient a, particularly due to comminution of the dosage form by mechanical action.

The following examples further illustrate the invention but are not to be construed as limiting its scope.

### General Operation Procedures

Powder mixtures of various ingredients were manufactured by weighing (10 kg balance), sieving (1.0 mm hand sieve) and blending. The thus obtained powder mixtures were then hot-melt extruded (twin-screw extruder, Leistritz ZSE 18, blunt ends of kneading elements, and extrusion diameter of 8×0.8 mm). The extrudates were pelletized (LMP) and then analyzed.

In vitro dissolution was tested in accordance with USP (apparatus II), in 600 ml 0.1 M HCl (pH 1) at 75 rpm (n=3).

Resistance against solvent extraction was tested by dispensing particle(s) A in 5 ml of boiling water. After boiling for 5 minutes the liquid was drawn up into a syringe (needle 21 G equipped with a cigarette filter), and the amount of the pharmacologically active ingredient a contained in the liquid within the syringe was determined via HPLC.

### PREPARATION EXAMPLE A

### Tamper-Resistant Hot-Melt Extruded Hydrocodon Particles—Particle(s) A

Powder mixtures of various ingredients were manufactured by weighing (10 kg balance), sieving (1.0 mm hand sieve) and blending. The thus obtained powder mixtures were then hot-melt extruded (twin-screw extruder, Leistritz ZSE 18, blunt ends of kneading elements, and extrusion diameter of 8×0.8 mm). The extrudates were pelletized (LMP) and then analyzed.

Powder mixtures of the following ingredients were manufactures and subsequently hot-melt extruded (1500 g particles, 180 mg per particle) under the following extrusion conditions:

| | A | |
|---|---|---|
| per particle | [mg] | [wt.-%] |
| Hydrocodone bitartrate | 10.00 | 5.56 |
| citric acid | 1.44 | 0.80 |
| polyethylene glycol (PEG6000) | 25.20 | 14.00 |
| α-tocopherol (as PEG blend, 14 wt.-% α-tocopherol, | 0.36 | 0.20 |
| ISP) | | |
| Carbopol 71G | 36.00 | 20.00 |
| polyethylene oxide (PEO 7 Mio) | 107.00 | 59.44 |
| | 180.00 | 100.00 |

| | |
|---|---|
| Speed screw [rpm] | 100 |
| Feed rate [g/min] | 16.66 |
| Melt pressure [bar] | 110 |
| melt temperature discharge [° C.] | 142 |

### PREPARATION EXAMPLE B

### Tamper-Resistant Hot-Melt Extruded Acetaminophen Particles—Particle(s) B

Powder mixtures of various ingredients were manufactured by weighing (10 kg balance), sieving (1.0 mm hand sieve) and blending. The thus obtained powder mixtures were then hot-melt extruded (twin-screw extruder, Leistritz ZSE 18, blunt ends of kneading elements, and extrusion diameter of 8×0.8 mm). The extrudates were pelletized (LMP) and then analyzed.

Powder mixtures of the following ingredients were manufactures and subsequently hot-melt extruded (500 g particles, 180 mg per particle) under the following extrusion conditions:

| | B | |
|---|---|---|
| per particle | [mg] | [wt.-%] |
| Acetaminophen | 18.00 | 10.00 |
| citric acid | 1.44 | 0.80 |
| polyethylene glycol (PEG6000) | 18.00 | 10.00 |
| α-tocopherol (as PEG blend, 14 wt.-% α-tocopherol, | 0.36 | 0.20 |
| ISP) | | |
| Carbopol 71G | 30.60 | 17.00 |
| polyethylene oxide (PEO 7 Mio) | 111.60 | 62.00 |
| | 180.00 | 100.00 |

| | |
|---|---|
| Speed screw [rpm] | 100 |
| Feed rate [g/min] | 17.60 |
| Melt pressure [bar] | 104 |
| melt temperature discharge [° C.] | 139.8 |

### PREPARATION EXAMPLES C AND D

### Tamper-Resistant Hot-Melt Extruded Hydrocodone/Acetaminophen Particles—Particle(s) A

Powder mixtures of various ingredients were manufactured by weighing (10 kg balance), sieving (1.0 mm hand sieve) and blending. The thus obtained powder mixtures were then hot-melt extruded (twin-screw extruder, Leistritz ZSE 18, blunt ends of kneading elements, and extrusion diameter of 8×0.8 mm). The extrudates were pelletized (LMP) and then analyzed.

Powder mixtures of the following ingredients were manufactures and subsequently hot-melt extruded (500 g particles, 180 mg per particle) under the following extrusion conditions:

| | C | | D | |
|---|---|---|---|---|
| per particle | [mg] | [wt.-%] | [mg] | [wt-%] |
| Hydrocodone bitartrate | 10.00 | 5.56 | 10.00 | 5.56 |
| Acetaminophen | 18.00 | 10.00 | 18.00 | 10.00 |
| citric acid | 1.44 | 0.80 | 1.44 | 0.80 |
| polyethylene glycol (PEG6000) | 18.00 | 10.00 | 18.00 | 10.00 |
| α-tocopherol (as PEG blend, | 0.36 | 0.20 | 0.36 | 0.20 |
| 14 wt.-% α-tocopherol, ISP) | | | | |
| Carbopol 71G | 30.60 | 17.00 | 30.60 | 17.00 |
| xanthan | — | — | 9.00 | 5.00 |
| polyethylene oxide (PEO 7 Mio) | 101.60 | 56.44 | 92.60 | 51.44 |
| | 180.00 | 100.00 | 180.00 | 100.00 |

| | | |
|---|---|---|
| Speed screw [rpm] | 100 | 100 |
| Feed rate [g/min] | 16.48 | 16.37 |
| Melt pressure [bar] | 133 | 136 |
| melt temperature discharge [° C.] | 138.2 | 138.1 |

### EXAMPLES

Various pharmaceutical dosage forms were manufactured from the intermediate products obtained in preparation examples A to D and powdery acetaminophen by filling well defined amounts into hard gelatine capsules of different size.

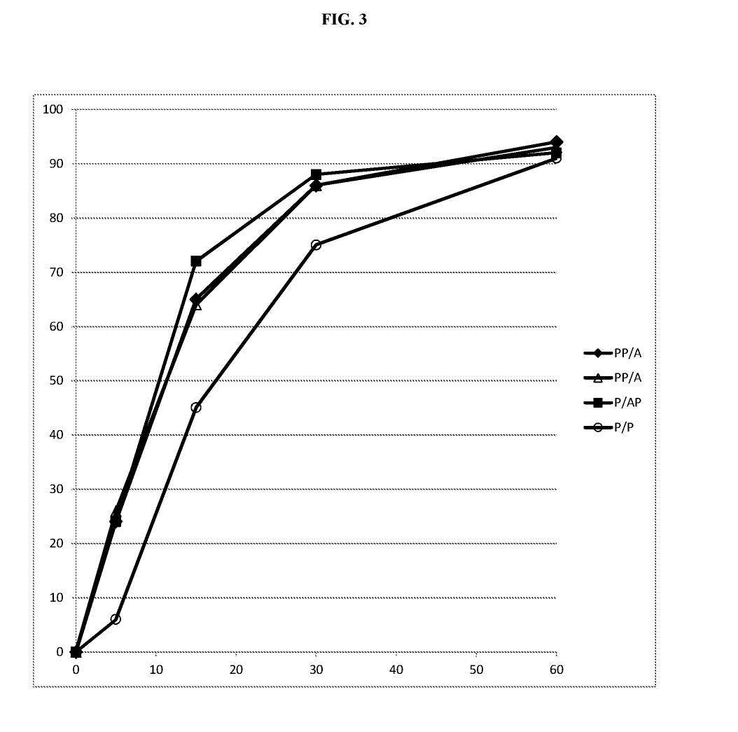

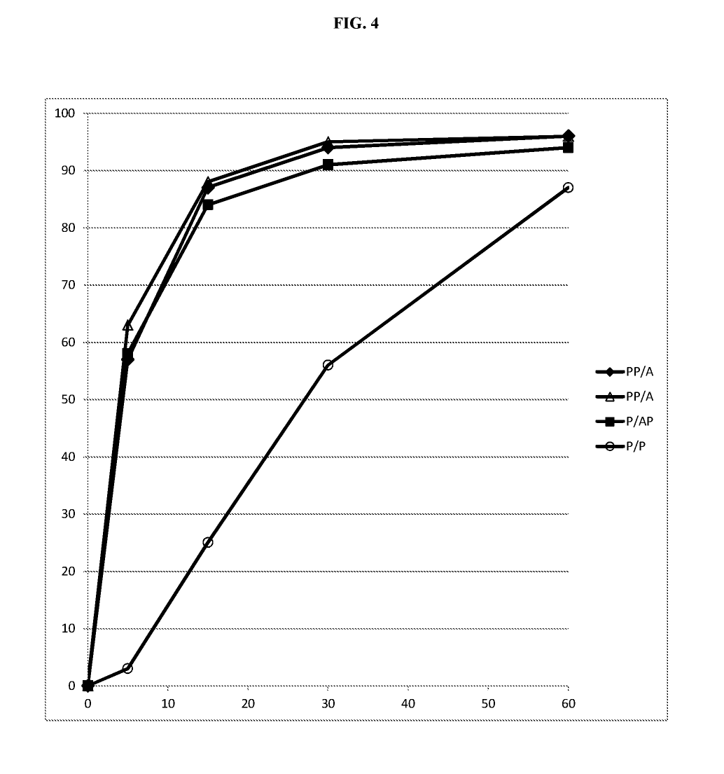

The in vitro release profiles of these dosage forms were measured. The individual composition of the dosage forms as well as the results of the in vitro release measurements are shown in the table here below. The in vitro release profiles with respect to the release of hydrocodone (pharmacologically active ingredient a) are shown in FIG. 3. The in vitro release profiles with respect to the release of acetaminophen (pharmacologically active ingredient b) are shown in FIG. 4.

| | | | |
|---|---|---|---|
| 1 | 2 | 3 | Comp.-4 |
| PP/A | PP/A | P/AP | P/P |

Component per capsule [mg]
{Hyrocodon//Acetaminophen, [mg]//[mg]}

| | | | | |
|---|---|---|---|---|
| Hydrocodon particles according to | — | — | 180.0 | 180.0 |
| preparation example A | | | {10.0//—} | {10.0//—} |
| Acetaminophen particles according to | — | — | 180.0 | 360.0 |
| preparation example B | | | {—//18.0} | {—//36.0} |
| Hydrocodon/Acetaminophen particles | 180.0 | — | — | — |
| according to preparation example C | {10.0//18.0} | | | |
| Hydrocodon/Acetaminophen particles | — | 180.0 | — | — |
| according to preparation example D | | {10.0//18.0} | | |
| Acetaminophen API powder | 307.0 | 307.0 | 307.0 | — |

Hard gelatine capsule:

| | | | | |
|---|---|---|---|---|
| size 0EL | + | + | | |
| size 00 | | | + | + |
| Dissolution % (0.1N HCl) | PP/A | PP/A | P/AP | P/P |

Hydrocodon:

| | | | | |
|---|---|---|---|---|
| after 5 min | 24 | 26 | 24 | 6 |
| after 15 min | 65 | 64 | 72 | 45 |
| after 30 min | 86 | 86 | 88 | 75 |
| after 60 min | 94 | 93 | 92 | 91 |

Acetaminophen:

| | | | | |
|---|---|---|---|---|
| after 5 min | 57 | 63 | 58 | 3 |
| after 15 min | 87 | 88 | 84 | 25 |
| after 30 min | 94 | 95 | 91 | 56 |
| after 60 min | 96 | 96 | 94 | 87 |

It becomes clear from the above data that when providing pharmacologically active ingredient b in form of a powder, a fast release thereof can be achieved.

The capsules according to Examples 1 and 2 according to the invention contained portion b_{A} of the pharmacologically active ingredient b in particles A (180 mg particles A containing 18.0 mg acetaminophen) and portion b_{P} of the pharmacologically active ingredient b (307.0 mg acetaminophen) in form of a powder outside particles A. As shown in FIGS. 3 and 4, both capsules show excellent release of both pharmacologically active ingredients a and b.

The capsules according to Examples 3 according to the invention contained portion b_{B} of the pharmacologically active ingredient b in particles B (180 mg particles B containing 18.0 mg acetaminophen) and portion b_{P} of the pharmacologically active ingredient b (307.0 mg acetaminophen) in form of a powder outside particles A and outside particles B. As shown in FIGS. 3 and 4, this capsule also shows excellent release of both pharmacologically active ingredients a and b.

The capsule according to comparative Example 4 did not contain the pharmacologically active ingredient b in form of a powder outside particles A. Instead, compared to Example 3 a higher content of particles B was included (360 mg particles B containing 36 mg acetaminophen). While these capsules according to comparative still provide rapid release of the comparative low amount of acetaminophen, the release provided by the capsules according to inventive Examples 1 to 3 is significantly faster.

### EXAMPLE E

### Quantity of Disintegrant Part I

The influence of the optionally present disintegrant in the particles was investigated. Compositions E-1 to E-3 were prepared and in vitro dissolution as well as resistance against solvent extraction were determined.

| | E-1 | | E-2 | | E-3 | |
|---|---|---|---|---|---|---|
| Substance per dose | mg | wt.-% | mg | wt.-% | mg | wt.-% |
| Oxycodone HCl | 10.00 | 5.56 | 10.00 | 5.56 | 10.00 | 5.56 |
| Citric acid | 1.44 | 0.80 | 1.44 | 0.80 | 1.44 | 0.80 |
| PEG 6000 | 27.51 | 15.28 | 25.20 | 14.00 | 27.51 | 15.28 |
| α-Tocopherol | 0.36 | 0.20 | 0.36 | 0.20 | 0.36 | 0.20 |
| Xanthan Gum | 9.00 | 5.00 | 9.00 | 5.00 | 9.00 | 5.00 |
| Type 602 | | | | | | |
| PEO 7 Mio. | 104.69 | 58.16 | 98.00 | 54.44 | 91.31 | 50.73 |
| Sodium starch | 27.00 | 15.00 | 36.00 | 20.00 | 45.00 | 25.00 |
| glycolate | | | | | | |
| | 180.00 | 100.00 | 180.00 | 100.00 | 180.00 | 100.00 |
| Dissolution | | | | | | |
| (n = 3): | | | | | | |

| | | | |
|---|---|---|---|
| 0 | 0.00 | 0.00 | 0.00 |
| 5 | 64.46 | 69.73 | 62.04 |
| 15 | 78.42 | 87.57 | 81.83 |
| 30 | 91.24 | 94.44 | 91.76 |
| 60 | 94.82 | 96.49 | 95.12 |
| extraction without | | | |
| milling: | | | |
| mean [%] | 10.10 | 0.00* | 16.37 |
| SD [%] | 4.67 | 0.00* | 12.67 |

*not tested, sample too jelly and could not be drawn into syringe

It becomes clear from the above comparative data that under the given conditions the best results could be achieved at a content of 20 wt.-% disintegrant (here sodium starch glycolate).

### EXAMPLE F

### Quantity of Disintegrant Part II

The influence of the optionally present disintegrant in the particles was investigated. Compositions F-1 to F-4 were prepared and in vitro dissolution as well as resistance against solvent extraction were determined.

| | F-1 | | F-2 | | F-3 | | F-4 | |
|---|---|---|---|---|---|---|---|---|
| per dose | mg | wt.-% | mg | wt.-% | mg | wt.-% | mg | wt.-% |
| Amphetamine sulfate | 30.00 | 13.95 | 30.00 | 16.67 | 30.00 | 13.95 | 30.00 | 16.67 |
| PEG 6000 | 27.20 | 12.65 | 21.85 | 12.14 | 27.20 | 12.65 | 21.85 | 12.14 |
| α-Tocopherol | 0.43 | 0.20 | 0.36 | 0.20 | 0.43 | 0.20 | 0.36 | 0.20 |
| Polyethylene oxide | 114.37 | 53.20 | 91.79 | 50.99 | 114.37 | 53.20 | 91.79 | 50.99 |
| 7 Mio. | | | | | | | | |
| Croscarmellose | 43.00 | 20.00 | 36.00 | 20.00 | | | | |
| sodium | | | | | | | | |
| Starch 1500 | | | | | 43.00 | 20.00 | 36.00 | 20.00 |
| Σ | 215.00 | 100.00 | 180.00 | 100.00 | 215.00 | 100.00 | 180.00 | 100.00 |

| | | | | |
|---|---|---|---|---|
| Speed screw [rpm] | 100 | 100 | 100 | 100 |
| Extruder Load [%] | 75.00 | 75.00 | 75.00 | 75.00 |
| Melt pressure [bar] | 1 | 1 | 1 | 1 |
| melt temperature | 145 | 145 | 145 | 145 |
| discharge [° C.] | | | | |

The in vitro dissolution test revealed the following release profiles:

Dissolution Amphetamine sulfate %

| | F-1 | F-2 | F-3 | F-4 |
|---|---|---|---|---|
| after 5 min | 60 | 74 | 75 | 78 |
| after 15 min | 91 | 94 | 82 | 81 |
| after 30 min | 97 | 99 | 84 | 87 |
| after 60 min | 97 | 99 | 85 | 88 |

The test for tamper-resistance provided the following results (where all tested pellets remained intact after the breaking strength tester had reached its upper force limit):

| test battery | F-1 | F-2 | F-3 | F-4 |
|---|---|---|---|---|
| 1 | 7.92 | 17.51 | 0.00* | 6.42 |
| 2 | 7.74 | 12.79 | 0.00* | 3.66 |
| 3 | 8.49 | 16.85 | 0.00* | 1.83 |
| mean [%] | 8.05 | 15.72 | 0.00* | 3.97 |
| SD [%] | 0.39 | 2.56 | 0.00* | 2.31 |

*not tested, sample too jelly and could not be drawn into syringe

It becomes clear from the above comparative data that under the given conditions lower contents of disintegrant provide an improved resistance against solvent extraction.

1. A tamper-resistant pharmaceutical dosage form comprising a pharmacologically active ingredient a having a psychotropic effect and a pharmacologically active ingredient b;
wherein
at least a portion of the pharmacologically active ingredient a is contained in one or more particles A which comprise a polymer matrix in which the pharmacologically active ingredient a is embedded; and
at least a portion of the pharmacologically active ingredient b is contained outside the one or more particles A in form of a powder; and
wherein the dosage form releases under in vitro conditions after 30 min
at least 50 wt.-% of the pharmacologically active ingredient a originally contained in the dosage form; and/or
at least 50 wt.-% of the pharmacologically active ingredient b originally contained in the dosage form.

2. The dosage form according to claim 1, wherein the release is measured in 600 ml 0.1 M HCl, pH 1 and at 75 rpm using an USP apparatus II.

3. The dosage form according to claim 1, wherein the pharmacologically active ingredient a is an active ingredient with potential for being abused.

4. The dosage form according to claim 1, wherein the pharmacologically active ingredient a is selected from the group consisting of opiates, opioids, stimulants, tranquilizers, and other narcotics.

5. The dosage form according to claim 1, wherein the pharmacologically active ingredient a is an opioid selected from the group consisting of natural opium alkaloids, phenylpiperidine derivatives, diphenylpropylamine derivatives, benzomorphan derivatives, oripavine derivatives and morphinan derivatives.

6. The dosage form according to claim 1, wherein the pharmacologically active ingredient a is an opioid selected from the group consisting of oxycodone, hydrocodone, oxymorphone, hydromorphone, morphine, tramadol, tapentadol, cebranopadol and the physiologically acceptable salts thereof.

7. The dosage form according to claim 1, wherein the pharmacologically active ingredient a is a physiologically acceptable salt of hydrocodone; or the pharmacologically active ingredient a is hydrocodone bitartrate; or the pharmacologically active ingredient a is hydrocodone or a physiologically acceptable salt thereof and the interval of time (tₘₐₓ) from administration of the active ingredient until the maximum plasma concentration (Cₘₐₓ) of the active ingredient is reached is within the range of 1.3±1.2 h.

8. The dosage form according to claim 1, wherein the pharmacologically active ingredient a is a physiologically acceptable salt of oxycodone; or the pharmacologically active ingredient a is oxycodone hydrochloride; or the pharmacologically active ingredient a is oxycodone or a physiologically acceptable salt thereof and the interval of time (tₘₐₓ) from administration of the active ingredient until the maximum plasma concentration (Cₘₐₓ) of the active ingredient is reached is within the range of 2.6±2.5 h.

9. The dosage form according to claim 1, wherein the pharmacologically active ingredient b is a non-opioid analgesic.

10. The dosage form according to claim 1, wherein the pharmacologically active ingredient b is selected from the group consisting of ATC classes [M01A], [M01C], [N02B] and [N02C] according to the WHO as follows:
ATC class [M01A] according to the WHO is selected from the group consisting of butylpyrazolidines, acetic acid derivatives, oxicams, propionic acid derivatives, fenamates, coxibs, nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate, avocado and soyabean oil, unsaponifiables, and feprazone;
ATC class [M01C] according to the WHO is selected from the group consisting of quinolines, gold preparations and penicillamine and buccilamine;
ATC class [N02B] according to the WHO is selected from the group consisting of salicylic acid and derivatives thereof, pyrazolones, anilides, rimazolium, glafenine, floctafenine, viminol, nefopam, flupirtine, ziconotide, methoxyflurane and cannabinoids; and
ATC class [N02C] according to the WHO is selected from the group consisting of ergot alkaloids, corticosteroid derivatives, selective serotonin (5HT1) agonists, pizotifen, clonidine, iprazochrome, dimetotiazine, oxetorone.

11. The dosage form according to claim 1, wherein the pharmacologically active ingredient b is acetaminophen or ibuprofen.

12. The dosage form according to claim 1, wherein the relative weight ratio of the pharmacologically active ingredient b to the pharmacologically active ingredient a is within the range of from 10:1 to 150:1.

13. The dosage form according to claim 1, wherein the pharmacologically active ingredient a is hydrocodone or a physiologically acceptable salt thereof and the pharmacologically active ingredient b is acetaminophen.

14. The dosage form according to claim 1, wherein the pharmacologically active ingredient a is oxycodone or a physiologically acceptable salt thereof and the pharmacologically active ingredient b is acetaminophen.

15. The dosage form according to claim 1, wherein the one or more particles A amount to a total number within the range of from 20 to 600; or the one or more particles A are made from the same mixture of ingredients and/or are substantially of the same size, shape, weight and composition; or the one or more particles A have an average individual weight within the range of from 0.1 mg to 5 mg; or the one or more particles A have a total weight within the range of from 10 mg to 500 mg; or the one or more particles A amount to a total content within the range of from 10 wt.-% to 80 wt.-%, based on the total weight of the dosage form; or the one or more particles A are tamper-resistant as such so that they also provide tamper-resistance after they have been separated from the remaining constituents of the dosage form; or the one or more particles A have a breaking strength of at least 300 N; or the one or more particles A contain the total amount of the pharmacologically active ingredient a that is contained in the dosage form; or the one or more particles A comprise only a single pharmacologically active ingredient a; or the one or more particles A comprise a combination of two or more pharmacologically active ingredients a; or the one or more particles A comprise additional pharmaceutical excipients selected from the group consisting of disintegrants, antioxidants and plasticizers; or the one or more particles A are thermoformed by hot-melt extrusion; or the one or more particle(s) A after 30 min under in vitro conditions in 600 ml 0.1 M HCl at pH 1 and at 75 rpm using an USP apparatus II release
at least 80 wt.-% of the pharmacologically active ingredient a that was originally contained in particle(s) A, and/or
at least 80% of the pharmacologically active ingredient b originally contained in particle(s) A.

16. The dosage form according to claim 1, wherein the polymer matrix comprises a polyalkylene oxide.

17. The dosage form according to claim 16, wherein the polymer matrix comprises a polyalkylene oxide selected from polymethylene oxide, polyethylene oxide and polypropylene oxide, or copolymers thereof; or the polymer matrix comprises polyethylene oxide; or the polymer matrix comprises a polyalkylene oxide having an average molecular weight of at least 200,000 g/mol; or the polymer matrix comprises a polyalkylene oxide having an average molecular weight in the range of 1,000,000 g/mol to 15,000,000 g/mol; or the overall content of the polyalkylene oxide is in the range of at least 25 wt.-% based on the total weight of the particle(s) A; or the overall content of the polyalkylene oxide is within the range of from 25 to 80 wt.-% based on the total weight of the dosage form and/or based on the total weight of the particle(s) A; or the overall content of the polyalkylene oxide is in the range of 50±20 wt.-% based on the total weight of the dosage form and/or based on the total weight of the particle(s) A.

18. The dosage form according to claim 1, wherein the total amount of the pharmacologically active ingredient b is contained outside the particles A in form of a powder.

19. The dosage form according to claim 1, wherein a portion b_{P} of the pharmacologically active ingredient b is contained outside the particles A in form of a powder and wherein a portion b_{A} of the pharmacologically active ingredient b is contained in particles A; or a portion by of the pharmacologically active ingredient b is contained outside the particles A in form of a powder and wherein a portion b_{C} of the pharmacologically active ingredient b is contained in a coating of particles A; or a portion b_{P} of the pharmacologically active ingredient b is contained outside the particles A in form of a powder and wherein a portion b_{B} of the pharmacologically active ingredient b is contained in one or more particles B differing from particles A; or a portion b_{P} of the pharmacologically active ingredient b is contained outside the particles A in form of a powder and wherein a portion b_{G} of the pharmacologically active ingredient b is contained outside particles A in form of granules.

20. The dosage form according to claim 19, wherein the one or more particles B each comprise a polymer matrix in which the portion b_{B} of the pharmacologically active ingredient b is embedded; or the one or more particles B each have a breaking strength of at least 300 N.

21. The dosage form according to claim 19, wherein the relative weight ratio of portion b_{P} to portion b_{A}, of portion b_{P} to portion b_{B}, of portion b_{P} to portion b_{C}, and of portion b_{P} to portion b_{G}, respectively, is within the range of from 100:1 to 1:100.

22. The dosage form according to claim 21, wherein the portion b_{P} consists of the pharmacologically active ingredient b; or the content of portion b_{P} is at least 70 wt.-% relative to the total content of the pharmacologically active ingredient b that is contained in the dosage form; or the dosage form releases under in vitro conditions after 30 min least 80 wt.-% of portion b_{P}.

23. The dosage form according to claim 19, wherein the dosage form is a capsule wherein the capsule filling contains the portion b_{P} together with particle(s) A and/or particle(s) B, granules and optionally further ingredients.

24. The dosage form according to claim 23, wherein the capsule filling contains further ingredients selected from the group consisting of fillers, binders, dispersing agents, wetting agents, disintegrants, gelling agents, antioxidants, preservatives, lubricants and plasticizers.

25. The dosage form according to claim 24, wherein the filler or binder selected from the group consisting of silicium dioxide, microcrystalline cellulose; cellulose ether; mannitol; dextrines; dextrose; calciumhydrogen phosphate; tricalcium phosphate, maltodextrine; lactose; polyvinylpyrrolidone; saccharose; magnesium salts; starches; pretreated starches; alginates and chitosanes; or the filler or binder material is lactose; or the filler or binder material is mannitol; or the content of the filler or binder is within the range of 35±30 wt.-% based on the total weight of the dosage form; or the content of the filler or binder is within the range of 65±30 wt.-% based on the total weight of the dosage form.

26. The dosage form according to claim 19, wherein the particle(s) A and/or the particle(s) B comprise a disintegrant.

27. The dosage form according to 26, wherein the particle(s) A and/or the particle(s) B comprise a disintegrant selected from polysaccharides, starches, starch derivatives, cellulose derivatives, polyvinylpyrrolidones, acrylates, gas releasing substances, and the mixtures of any of the foregoing; or the particle(s) A and/or the particle(s) B comprise croscarmellose as a disintegrant; or the particle(s) A and/or the particle(s) B comprise croscarmellose sodium and/or pregelatinized starch and/or sodium starch glycolate as a disintegrant; or the particle(s) A and/or the particle(s) B comprise a disintegrant at a content within the range of from 10 wt.-% to 20 wt.-% based on the total weight of the particle(s); or the particle(s) A and/or the particle(s) B comprise a disintegrant at a content of at least 12 wt.-% based on the total weight of the particle(s); or the particle(s) A and/or the particle(s) B comprise a disintegrant at a content of at least 15 wt.-% based on the total weight of the particle(s); or the particle(s) A and/or the particle(s) B comprise a disintegrant at a content of at least 20 wt.-% based on the total weight of the particle(s); or the particle(s) A and/or the particle(s) B comprise a disintegrant at a content within the range of from 20.00±6.00 wt.-% based on the total weight of the particle(s); or the particle(s) A and/or the particle(s) B comprise a disintegrant at a content within the range of from 15±3.0 wt.-% based on the total weight of the particle(s).

28. The dosage form according to claim 1, wherein the dosage form is a capsule or a tablet.

29. A method of treating pain in a patient, said method comprising administering to said patient at least one dosage form according to claim 1 in a quantity and for a period of time effective to treat pain.
